(19) United States
(12) Patent Application Publication
(10) Pub. No.: US 2010/0292210 A1
(43) Pub. Date: Nov. 18, 2010

(54) Novel Compounds for A-Beta-Related Pathologies

(75) Inventors: Yvonne Lo-Alfredsson, Sodertalje (SE); Kim Paulsen, Sodertälje (SE); Laszlo Rakos, Södertalje (SE); Didier Rotticci, Sodertalje (SE); Magnus Waldman, Södertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/777,602

(22) Filed: May 11, 2010

Related U.S. Application Data
(60) Provisional application No. 61/177,305, filed on May 12, 2009.
(60) Provisional application No. 61/289,600, filed on Dec. 23, 2009.

(51) Int. Cl. A61K 31/553; C07D 267/08; A61P 25/28

(52) U.S. Cl. 514/211.09; 540/552

(57) ABSTRACT

The present invention relates to novel compounds of formulae I and II and therapeutically acceptable salts thereof, their pharmaceutical compositions processes for making them and their use in therapeutic methods for treatment and/or prevention of various diseases. In particular, the invention relates to compounds which interfere with γ-secretase and/or its substrate and hence modulate the formation of Aβ peptides.

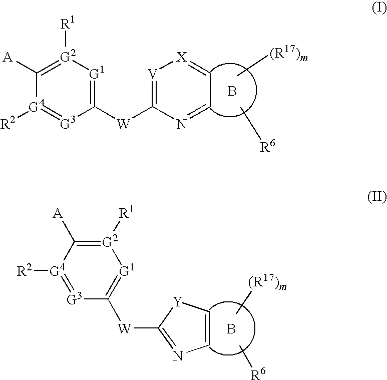

### TECHNICAL FIELD OF THE INVENTION

The present invention relates to novel compounds and pharmaceutically acceptable salts thereof. Furthermore, the present invention also relates to pharmaceutical compositions comprising said compounds, processes for making said compounds and their use as medicaments for treatment and or prevention of various diseases. In particular, the present invention relates to compounds, which interfere with γ-secretase and or its substrate and hence modulate the formation of Aβ peptides. These compounds are used for treatment and or prevention of Aβ-related pathologies, such as Alzheimer's disease, Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer's disease, memory loss, attention deficit symptoms associated with Alzheimer's disease, neurodegeneration associated with diseases such as Alzheimer's disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

The prime neuropathological event distinguishing Alzheimer's disease (AD) is deposition of the amyloid β-peptide (Aβ) in brain parenchyma and cerebral vessels. A large body of genetic, biochemical and in vivo data support a pivotal role for Aβ in the pathological cascade that eventually leads to AD. Patients usually present early symptoms (commonly memory loss) in their sixth or seventh decades of life. The disease progresses with increasing dementia and elevated deposition of Aβ. In parallel, a hyperphosphorylated form of the microtubule-associated protein tau accumulates within neurons, leading to a plethora of deleterious effects on neuronal function. The prevailing working hypothesis regarding the temporal relationship between Aβ and tau pathologies states that Aβ deposition precedes tau aggregation in humans and animal models of the disease. Within this context, it is worth noting that the exact molecular nature of Aβ, mediating this pathological function is presently an issue under intense study. Most likely, there is a continuum of toxic species ranging from lower order Aβ oligomers to supramolecular assemblies such as Aβ fibrils.

The Aβ peptide is an integral fragment of the Type I protein APP (Aβ amyloid precursor protein), a protein ubiquitously expressed in human tissues. Aβ can be found in both plasma, cerebrospinal fluid (CSF), and in the medium from cultured cells, and is generated as a result of APP proteolysis. There are two main cleavages of APP that results in Aβ production, the so-called β-, and γ-cleavages. The β-cleavage, which generates the N terminus of Aβ, is catalyzed by the transmembrane aspartyl protease BACE1. The γ-cleavage, generating the Aβ C termini and subsequent release of the peptide, is effected by a multi-subunit aspartyl protease named γ-secretase. Both BACE1 and γ-secretase processes APP at different sites, resulting in Aβ peptides of different lengths and heterologous N- and C-termini. The invention described herein covers all N-terminal variants of Aβ. Therefore, for the sake of simplicity, all N-terminal variants will be is covered by the denotation Aβ.

The activity of γ-secretase causes the liberation of many Aβ peptides, such as Aβ37, Aβ38, Aβ39, Aβ40, Aβ42 and Aβ43, of which Aβ40 is the most common. These peptides show a different propensity to aggregate, and in particular Aβ42 is prone to form oligomers and fibrillar deposits. Intriguingly, human genetics strongly support a key role for Aβ42 as a key mediator of Alzheimer pathogenesis. Indeed, more than 150 different mutations causing familial Alzheimer's disease either result in an increase in the ratio of Aβ 42/40 peptides produced or affect the intrinsic aggregation behaviour of Aβ. Based on this knowledge, Aβ42 has become a prime target for therapeutic intervention in AD (Beher D, Curr Top Med Chem 2008; 8(1):34-7). Targeting Aβ42 at the level of γ-secretase activity must however be conducted with caution since γ-secretase catalyses proteolysis of many proteins, which have important physiological functions. Among its many substrates is the Notch receptor family, which signaling is essential for many different cell fate determination processes e.g. during embryogenesis and in the adult. As such, Aβ42 lowering strategies at the level of γ-secretase must be compatible with maintained Notch signaling.

Encouragingly, an enormous scientific effort and progress have suggested that it is indeed possible to combine γ-secretase interference and lowered Aβ42 production without obtaining toxic side effects due to impaired Notch signaling There have for instance been reports, which postulate that allosteric modulation of γ-secretase combines lowered Aβ42 production with maintained Notch signaling (Weggen et al. Nature 414(6860), 212-216 (2003)). In addition, a number of compounds interfering with γ-secretase and Aβ production have been suggested, in e.g. WO2005/054193, WO2005/001398, WO2004/073705, WO2007/135969, WO2007/139149, WO2005/115990, WO2008/097538, WO2008/099210, WO2008/100412 and WO2007/125364.

The present invention describes a new class of compounds, said compounds will inhibit the Aβ40 and 42 production, increase Aβ37 and Aβ38 levels and maintaining Notch signaling. These compounds will thus be useful in the prevention and/or treatment of Alzheimer's Disease (AD).

### SUMMARY OF THE INVENTION

It has been found that compounds of the Formulae (I) and (II), herein also referred to as the compounds of the (present) invention, are affecting the γ-secretase mediated processing of APP and thereby lowering the secretion of Aβ42 and Aβ40 peptides while causing an increase in the secreted levels of Aβ37 and Aβ38 and maintaining Notch signaling. These compounds can be used for treatment and/or prevention of Aβ-related pathologies.

In a first aspect, the invention relates to a compound of Formula (I) or (II)

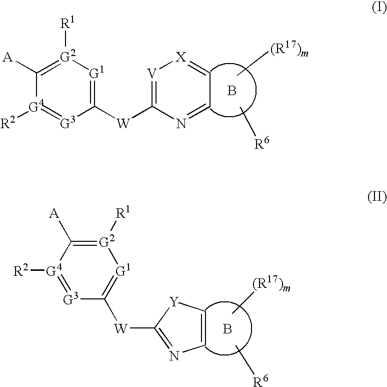

wherein
G¹, G², G³ and G⁴ are independently selected from nitrogen and carbon;
A is a 5 to 6 membered heteroaryl ring, wherein at least one of the ring forming atoms is selected from nitrogen and the remaining ring forming atoms are selected from carbon, nitrogen, sulphur and oxygen, wherein said heteroaryl is optionally substituted with one or more substituents selected from halogen, cyano, nitro, C₁₋₆alkyl, C₃₋₆cycloalkyl, C₂₋₆alkenyl, C₂₋₆alkynyl, SR⁴, N(R⁴)R⁵ and OR⁴, and wherein said C₁₋₆alkyl, C₂₋₆alkenyl or C₂₋₆alkynyl is optionally substituted with halogen, hydroxy, cyano, C₁₋₆alkoxy and OC₃₋₆cycloalkyl;
R¹ and R² are independently selected from hydrogen, halogen, hydroxy, cyano, nitro, C₁₋₆alkyl, C₂₋₆alkenyl, C₂₋₆alkynyl, C₃₋₆carbocyclyl, C₁₋₆alkoxy, OC₃₋₆carbocyclyl, N(R⁴)R⁵, N(R⁴)C(O)R⁵, N(R⁴)S(O)₂R⁵, C(O)R⁴, C(O)N(R⁴)R⁵, SR⁴, S(O)R⁴, S(O)₂R⁴ wherein said C₁₋₆alkyl, C₂₋₆alkenyl, C₂₋₆alkynyl, C₃₋₆carbocyclyl, C₁₋₆alkoxy or OC₃₋₆carbocyclyl is optionally substituted with one to three substituents selected from halogen, cyano, hydroxy, C₁₋₆alkoxy and OC₃₋₆carbocyclyl;
R¹ and said A ring may together form a ring;
X and V are independently selected from nitrogen or CR³;
R³ is selected from hydrogen, halogen, cyano, C₁₋₆alkyl, C₁₋₆alkoxy, OC₃₋₆carbocyclyl wherein said C₁₋₆alkyl, C₁₋₆alkoxy and OC₃₋₆carbocyclyl is optionally substituted with one or more halogen, cyano, hydroxy, SR⁴, S(O)R⁴, S(O)₂R⁴, N(R⁴)R⁵, N(R⁴)C(O)R⁵, C₁₋₆alkoxy, OCF₃, OCF₂H and OCFH₂;
R⁴ and R⁵ are independently selected from hydrogen, C₁₋₆alkyl wherein said C₁₋₆alkyl is optionally substituted with one or more substituents selected from halogen, OCF₃, OCF₂H and OCFH₂;
R⁴ and R⁵ may together form a 5 to 7 membered heterocycle ring containing one or more heteroatomes selected from N, O or S, wherein if said heterocyclic ring contains an nitrogen, that nitrogen may be optionally substituted by a group of substituents selected from C₁₋₆alkyl, C(O)C₁₋₆alkyl;
Y is selected from —N(R⁷)—, —S— and —O—;

### W is —N(R⁷)—, —C(R¹⁰)(R¹¹)— or —O—;

B is a saturated or partially unsaturated 5 to 7 membered ring, wherein two of the ring forming atoms are independently selected from nitrogen, sulfur and oxygen and the other ring forming atoms are carbon, and wherein a —CH₂— group can be optionally replaced by —C(O)— or —C(carbocyclyl)-;
is R⁶ is selected from C₁₋₁₀alkyl, aryl, heteroaryl, C₁₋₆alkylaryl, C₁₋₆alkylheteroaryl, C₄₋₁₀heterocyclyl, C₃₋₁₀-carbocyclyl; C₁₋₄alkylC₁₋₆heterocyclyl or C₁₋₄alkylC₃₋₁₀carbocyclyl wherein said C₁₋₁₀alkyl, aryl, heteroaryl, C₁₋₆alkylaryl, C₁₋₆alkylheteroaryl, C₄₋₁₀heterocyclyl, C₃₋₁₀carbocyclyl; C₁₋₄alkylC₁₋₆heterocyclyl or C₁₋₄alkylC₃₋₁₀carbocyclyl is optionally substituted with one or more substituents selected from halogen, cyano, hydroxy, nitro, C₁₋₆alkyl, C₂₋₆alkenyl, C₂₋₆alkynyl, C₁₋₆alkoxy, OC₃₋₆carbocyclyl, C₃₋₁₀carbocyclyl, heterocyclyl, SR⁷, S(O)R⁷, S(O)₂R⁷, S(O)₂N(R⁷)R⁸, C(O)R⁷, C(O)N(R⁷)R⁸, N(R⁷)R⁸, N(R⁹)C(O)N(R⁷)R⁸, N(R⁹)S(O)₂R⁷ and N(R⁷)C(O)R⁸; and wherein said C₁₋₆alkyl, C₂₋₆alkenyl, C₂₋₆alkynyl, C₁₋₆alkoxy, OC₃₋₆carbocyclyl, C₃₋₁₀carbocyclyl or heterocyclyl is optionally substituted with one or more substituents selected from cyano, hydroxy, halogen, OR⁷, S(O)₂R⁷, C(O)R⁷, C(O)N(R⁷)R⁸, N(R⁷)R⁸ and N(R⁹)C(O)N(R⁷)R⁸; R⁷, R⁸ and R⁹ are independently selected from hydrogen, C₄₋₈heterocyclyl and C₃₋₁₀carbocyclyl; C₁₋₁₀alkyl, C₁₋₆alkylOC₁₋₆alkyl, aryl or heteroaryl; wherein said C₄₋₁₀heterocyclyl, C₃₋₁₀carbocyclyl; C₃₋₁₀alkyl, C₁₋₆alkylOC₁₋₆alkyl, aryl and heteroaryl is optionally substituted with one or more substituents selected from halogen, cyano, hydroxy, OCF₃, OCF₂H, OCFH₂, C₁₋₆alkoxy, OC₃₋₆carbocyclyl and OC₄₋₆heterocyclyl;
R⁷ and R⁸ may together form a 5 to 7 membered heterocycle ring containing one or more heteroatomes selected from N, O or S, wherein if said heterocyclic ring contains an —NH-moiety that nitrogen may be optionally substituted by a group of substituents selected from C₄₋₈heterocyclyl and C₃₋₆carbocyclyl; C₁₋₆alkyl, C₁₋₁₀alkylOC₁₋₁₀alkyl, aryl, C(O)C₁₋₁₀alkyl or heteroaryl;
R¹⁰ and R¹¹ are independently selected from hydrogen, halogen, C₁₋₆alkyl, C₁₋₆alkoxy and C₁₋₆alkylOC₁₋₆alkyl;
R¹⁷ is independently selected from hydrogen, hydroxy, halogen, cyano, C₁₋₆alkyl, C₂₋₆alkenyl, C₂₋₆alkynyl, aryl, heteroaryl, C₁₋₆alkylaryl, C₁₋₆alkylheteroaryl, C₃₋₇carbocyclyl, heterocyclyl, OR⁷, SR⁷, S(O)R⁷, S(O)₂R⁷, S(O)₂N(R⁷)R⁸, N(R⁹)S(O)₂R⁷, N(R⁹)S(O)₂N(R⁷)R⁸, N(R⁷)R⁸, N(R⁷)C(O)R⁸, N(R⁹)C(O)N(R⁷)R⁸, C(O)R⁷ and C(O)N(R⁷)R⁸ wherein said C₁₋₆alkyl, C₂₋₆alkenyl, C₂₋₆alkynyl, aryl, heteroaryl, C₁₋₆alkylaryl, C₁₋₆alkylheteroaryl, C₃₋₇carbocyclyl, and heterocyclyl optionally is substituted with one or more substituents selected from cyano, hydroxy, halogen, OR⁷, S(O)₂R⁷, S(O)₂N(R⁷)R⁸, C(O)R⁷, C(O)N(R⁷)R⁸, N(R⁷)R⁸, N(R⁹)S(O)₂N(R⁷)R⁸ and N(R⁹)C(O)N(R⁷)R⁸; and
m is 2, 3 or 4;
or a pharmaceutically acceptable salt thereof
with the proviso that the compounds 8-(4-(1H-tetrazol-1-yl)phenoxy)-1,3,7-trimethyl-1H-purine-2,6(3H,7H)-dione and 8-(4-(1H-tetrazol-1-yl)phenoxy)-7-allyl-1,3-dimethyl-1H-purine-2,6(3H,7H)-dione are excluded.

In one embodiment of said first aspect of the invention, the compound is of Formula (III), or a pharmaceutically acceptable salt thereof:

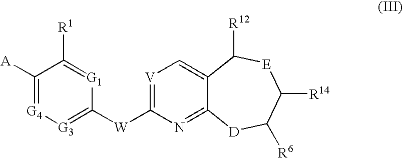

wherein
G₁, G₃, G₄ and V are independently selected from carbon and nitrogen;
A R¹, and R⁶ have the same meaning as previously defined;
W is selected from —NH— and —O—;
D is selected from —N(R¹⁶)— and —O—;
E is selected from —N(R¹³)—, —S(O)—, —S(O)₂— and —O—;
R¹³ when present is selected from hydrogen, C₁₋₆alkyl, C₁₋₆alkylOC₁₋₆alkyl, C₂₋₆alkenyl, C₂₋₆alkynyl, carbocyclyl, heterocyclyl, C(O)R⁷, C(O)N(R⁷)(R⁸), C(O)OR⁷, S(O)₂R⁷ and S(O)₂N(R⁷)(R⁸), wherein said C₁₋₆alkyl, C₁₋₆alkylOC₁₋₆alkyl, C₂₋₆alkenyl, C₂₋₆alkynyl, carbocyclyl or heterocyclyl is optionally substituted with one or more substituents selected from halogen, hydroxy, cyano, C(O)OR⁷, N(R⁸)(R⁸), C₁₋₆alkyl, C₁₋₆alkoxy, heterocyclyl and carbocyclyl and wherein R⁷ and R⁸ have the same meaning as defined above;
R¹⁶ when present, is selected from C₁₋₁₀alkyl, C₂₋₆alkenyl, C₂₋₆alkynyl, C₁₋₁₀carbocyclyl, heterocyclyl wherein said C₁₋₁₀alkyl, C₂₋₆alkenyl, C₂₋₆alkynyl, C₁₋₁₀carbocyclyl, heterocyclyl is optionally substituted with one or more substituents selected from cyano, hydroxy, and halogen; and
R¹² and R¹⁴ are independently selected from, hydrogen, ═O, cyano, OR⁷, SR⁷, S(O)R⁷, S(O)₂R⁷, S(O)₂N(R⁷)R⁸, N(R⁹)S(O)₂R⁷, N(R⁹)S(O)₂N(R⁷)R⁸, N(R⁷)R⁸, N(R⁷)C(O)R⁸, N(R⁹)C(O)N(R⁷)R⁸, C(O)R⁷, C(O)N(R⁷)R⁸, C₁₋₆alkyl, C₂₋₆alkenyl, C₂₋₆alkynyl, C₁₋₆alkoxy, aryl, heteroaryl, C₁₋₆alkylaryl, C₁₋₆alkylheteroaryl, C₄₋₁₀heterocyclyl, C₃₋₁₀carbocyclyl; C₁₋₄alkC₁₋₆heterocyclyl or C₁₋₄alkC₃₋₁₀carbocyclyl wherein said C₁₋₆alkyl, aryl, heteroaryl, C₁₋₆alkylaryl, C₁₋₆alkylheteroaryl, C₄₋₁₀heterocyclyl, C₃₋₁₀carbocyclyl; C₁₋₄alkC₁₋₆heterocyclyl or C₁₋₄alkC₃₋₁₀carbocyclyl is optionally substituted with one or more substituents selected from halogen, cyano, hydroxy, nitro, C₁₋₆alkyl, C₂₋₆alkenyl, C₂₋₆alkynyl, C₁₋₆alkoxy, OC₃₋₆carbocyclyl, C₃₋₁₀-carbocyclyl, heterocyclyl, SR⁷, S(O)R⁷, S(O)₂R⁷, S(O)₂N(R⁷)R⁸, C(O)R⁷, C(O)N(R⁷)R⁸, N(R⁷)R⁸, N(R⁹)C(O)N(R⁷)R⁸, N(R⁹)S(O)₂R⁷ and N(R⁷)C(O)R⁸; and wherein said C₁₋₆alkyl, C₂₋₆alkenyl, C₂₋₆alkynyl, C₁₋₆alkoxy, OC₃₋₆carbocyclyl, C₃₋₁₀carbocyclyl or heterocyclyl is optionally substituted with one or more substituents selected from cyano, hydroxy, halogen, OR⁷, S(O)₂R⁷, C(O)R⁷, C(O)N(R⁷)R⁸, N(R⁷)R⁸ and N(R⁹)C(O)N(R⁷)R⁸, wherein R⁷, R⁸ and R⁹ have the same meaning as previously defined.

In another embodiment of the first aspect of the invention is provided a compound of Formula (III), or a pharmaceutically acceptable salt thereof, wherein:

G₁, G₃, G₄, V, W, D and E has the same meaning as for the first embodiment of the first aspect of the invention;
A is a five-membered heteroaryl ring, wherein at least one of the ring forming atoms is is selected from nitrogen and the remaining ring forming atoms are selected from carbon, nitrogen, and oxygen, wherein said heteroaryl is optionally substituted with one or more substituents selected from halogen, cyano, nitro, C₁₋₆alkyl, and OR⁴, wherein R⁴ has the same meaning as previously defined;
R¹ is selected from hydrogen, halogen, hydroxy, cyano, nitro, C₁₋₆alkyl, and C₁₋₆alkoxy;
R⁶ is selected from C₁₋₁₀alkyl, aryl, heteroaryl, C₁₋₆alkylaryl, C₄₋₁₀heterocyclyl, C₃₋₁₀carbocyclyl, C₁₋₄alkC₁₋₆heterocyclyl or C₁₋₄alkC₃₋₁₀carbocyclyl wherein said C₁₋₁₀alkyl, aryl, heteroaryl, C₁₋₆alkylaryl, C₄₋₁₀heterocyclyl, C₃₋₁₀carbocyclyl; C₁₋₄alkC₁₋₆heterocyclyl or C₁₋₄alkC₃₋₁₀carbocyclyl is optionally substituted with one or more substituents selected from halogen, cyano, hydroxy, nitro, C₁₋₆alkoxy and C₁₋₆alkyl;
R¹² is selected from hydrogen and ═O;
R¹³, if present, is selected from hydrogen, C₁₋₆alkyl and —C(O)R⁷, wherein said C₁₋₆alkyl is optionally substituted with one or more substituents selected from halogen, cyano, hydroxy and C₁₋₆alkoxy, and wherein R⁷ has the same meaning as previously defined;
R¹⁴ is selected from hydrogen, ═O, hydroxy, halogen and C₁₋₆alkyl, wherein said C₁₋₆alkyl optionally is substituted with one or more substituents selected from halogen, cyano, hydroxy, and C₁₋₆alkoxy; and
R¹⁶ when present, is selected from substituents selected from hydrogen, C₁₋₁₀alkyl, C₃₋₁₀carbocyclyl wherein said C₁₋₁₀alkyl and C₃₋₁₀carbocyclyl is optionally substituted with one or more substituents selected from cyano, hydroxy, and halogen.

In another embodiment of the first aspect of the invention is provided a compound of Formula (III), or a pharmaceutically acceptable salt thereof, wherein:

G₁, G₃, G₄, V, W, D, E and R¹², has the same meaning as defined in relation to the second embodiment of the first aspect of the invention;
A is selected from imidazole, oxazole and oxadiazole, wherein said imidazole, oxazole and oxadiazole optionally is substituted with C₁₋₆alkyl, and preferably substituted with methyl;
R¹ is selected from hydrogen, halogen and C₁₋₆alkoxy;
R¹³, if present, is selected from C₁₋₆alkyl, —C(O)C₁₋₆alkyl and C₁₋₆hydroxyalkyl, wherein said C₁₋₆alkyl optionally is substituted with halogen;
R¹⁴ is selected from hydrogen, and ═O;
R⁶ is selected from phenyl, benzyl, tetrahydrofuranyl and cyclopentyl, wherein said phenyl, benzyl, tetrahydrofuranyl and cyclopentyl optionally are substituted with one or more substituents selected from halogen hydroxy, C₁₋₆alkoxy and cyano; and
R¹⁶, if present, is selected from hydrogen, methyl, and cyclopentyl.

In another embodiment of the first aspect of the invention is provided a compound of Formula (III), or a pharmaceutically acceptable salt thereof, wherein R⁶ is selected from phenyl, benzyl, tetrahydrofuranyl and cyclopentyl.

In another embodiment of the first aspect of the invention is provided a compound of Formula (III), or a pharmaceutically acceptable salt thereof, selected from:
N-[3-methoxy-4-(4-methyl-1H-imidazol-1-yl)phenyl]-4-methyl-2-phenyl-2,3,4,5-tetrahydropyrido[3,2-f][1,4]oxazepin-8-amine;
4-methyl-N-[4-(2-methyl-1H-imidazol-1-yl)phenyl]-2-phenyl-2,3,4,5-tetrahydropyrido[3,2-f][1,4]oxazepin-8-amine;
4-(2-hydroxyethyl)-8-(3-methoxy-4-(4-methyl-1H-imidazol-1-yl)phenylamino)-2-phenyl-3,4-dihydropyrido[3,2-f][1,4]oxazepin-5(2H)-one;
8-{[3-methoxy-4-(4-methyl-1H-imidazol-1-yl)phenyl]amino}-4-methyl-2-phenyl-3,4-dihydropyrido[3,2-f][1,4]oxazepin-5(2H)-one;
4-methyl-8-{[4(1,3-oxazol-5-yl)phenyl]amino}-2-phenyl-3,4-dihydropyrido[3,2-f][1,4]oxazepin-5(2H)-one;
4-methyl-N-(6-(2-methyl-1H-imidazol-1-yl)pyridin-3-yl)-2-phenyl-2,3,4,5-tetrahydropyrido[3,2-f][1,4]oxazepin-8-amine;
6,9-dimethyl-2-(4-(oxazol-5-yl)phenoxy)-8-phenyl-6,7,8,9-tetrahydro-5H-pyrimido[4,5-e][1,4]diazepin-5-one;
8-{[4-(1H-imidazol-1-yl)phenyl]amino}-4-methyl-2-phenyl-3,4-dihydropyrido[3,2-f][1,4]oxazepin-5(2H)-one;
4-methyl-8-{[4-(2-methyl-1H-imidazol-1-yl)phenyl]amino}-2-phenyl-3,4-dihydropyrido[3,2-f][1,4]oxazepin-5(2H)-one;
4-methyl-8-{[4-(3-methyl-1,2,4-oxadiazol-5-yl)phenyl]amino}-2-phenyl-3,4-dihydropyrido[3,2-f][1,4]oxazepin-5(2H)-one;
1-cyclopentyl-4-methyl-8-(4-(2-methyl-1H-imidazol-1-yl)phenylamino)-3,4-dihydro-1H-pyrido[2,3-e][1,4]diazepin-5(2H)-one;
2-(4-(1H-imidazol-1-yl)phenoxy)-6-methyl-8-phenyl-7,8-dihydropyrimido[5,4-f][1,4]oxazepin-5(6H)-one;
4-methyl-8-{[6-(2-methyl-1H-imidazol-1-yl)pyridin-3-yl]amino}-2-phenyl-3,4-dihydropyrido[3,2-f][1,4]oxazepin-5(2H)-one;
N-(4-(2-methyl-1H-imidazol-1-yl)phenyl)-2-phenyl-3,5-dihydro-2H-[1,4]dioxepino[5,6-b]pyridin-8-amine;
N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
N-(4-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
N-(6-methoxy-5-(4-methyl-1H-imidazol-1-yl)pyridin-2-yl)-2-phenyl-3,5-dihydro-2H-[1,4]dioxepino[5,6-b]pyridin-8-amine;
N-(6-methoxy-5-(4-methyl-1H-imidazol-1-yl)pyridin-2-yl)-4-methyl-2-(tetrahydrofuran-2-yl)-2,3,4,5-tetrahydropyrido[3,2-f][1,4]oxazepin-8-amine;
N-(3-methoxy-4-(4-methyl-1H-imidazol-1-yl)phenyl)-4-methyl-2-(tetrahydrofuran-2-yl)-2,3,4,5-tetrahydropyrido[3,2-f][1,4]oxazepin-8-amine;
4-(difluoromethyl)-N-(4-(2-methyl-1H-imidazol-1-yl)phenyl)-2-phenyl-2,3,4,5-tetrahydropyrido[3,2-f][1,4]oxazepin-8-amine;
1-(8-(4-(2-methyl-1H-imidazol-1-yl)phenylamino)-2-phenyl-2,3-dihydropyrido[3,2-f][1,4]oxazepin-4(5H)-yl)ethanone;
4-methyl-8-(4-(2-methyl-1H-imidazol-1-yl)phenylamino)-2-phenyl-4,5-dihydropyrido[3,2-f][1,4]oxazepin-3(2H)-one;
N-(3-methoxy-4-(4-methyl-1H-imidazol-1-yl)phenyl)-1,4-dimethyl-2-phenyl-2,3,4,5-tetrahydro-1H-pyrido[2,3-e][1,4]diazepin-8-amine;
1,4-dimethyl-8-(4-(2-methyl-1H-imidazol-1-yl)phenylamino)-2-phenyl-4,5-dihydro-1H-pyrido[2,3-e][1,4]diazepin-3(2H)-one;
2-(3-fluorophenyl)-N-(4-methoxy-5-(4-methyl-1H-imidazol-1-yl)pyridin-2-yl)-4-methyl-2,3,4,5-tetrahydropyrido[3,2-f][1,4]oxazepin-8-amine;
N-(3-methoxy-4-(4-methyl-1H-imidazol-1-yl)phenyl)-6-methyl-8-phenyl-5,6,7,8-tetrahydropyrimido[5,4-f][1,4]oxazepin-2-amine;
N-(3-methoxy-4-(4-methyl-1H-imidazol-1-yl)phenyl)-1,4-dimethyl-2-phenyl-2,3,4,5-tetrahydro-1H-pyrido[2,3-e][1,4]diazepin-8-amine;
N-[3-methoxy-4-(4-methyl-1H-imidazol-1-yl)phenyl]-8-phenyl-7,8-dihydro-5H-[1,4]oxathiepino[7,6-d]pyrimidin-2-amine6,6-dioxide;
N-[3-methoxy-4-(4-methyl-1H-imidazol-1-yl)phenyl]-8-phenyl-7,8-dihydro-5H-[1,4]oxathiepino[7,6-d]pyrimidin-2-amine6-oxide;
N-(3-fluoro-4-(4-methyl-1H-imidazol-1-yl)phenyl)-6-methyl-8-phenyl-5,6,7,8-tetrahydropyrimido[5,4-f][1,4]oxazepin-2-amine;
N-(3-fluoro-4-(4-methyl-1H-imidazol-1-yl)phenyl)-6-(2-methoxyethyl)-8-phenyl-5,6,7,8-tetrahydropyrimido[5,4-f][1,4]oxazepin-2-amine;
(R)—N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
(R)—N-(3-methoxy-4-(4-methyl-1H-imidazol-1-yl)phenyl)-4-methyl-2-phenyl-2,3,4,5-tetrahydropyrido[3,2-f][1,4]oxazepin-8-amine;
(R)-1-(8-(6-methoxy-5-(4-methyl-1H-imidazol-1-yl)pyridin-2-ylamino)-2-phenyl-2,3-dihydropyrido[3,2-f][1,4]oxazepin-4(5H)-yl)ethanone;
(R)-6-(2-fluoroethyl)-N-(3-methoxy-4-(4-methyl-1H-imidazol-1-yl)phenyl)-8-phenyl-5,6,7,8-tetrahydropyrimido[5,4-f][1,4]oxazepin-2-amine;
(R)—N-(3-methoxy-4-(4-methyl-1H-imidazol-1-yl)phenyl)-6-methyl-8-phenyl-5,6,7,8-tetrahydropyrimido[5,4-f][1,4]oxazepin-2-amine;
2-(8-(3-methoxy-4-(4-methyl-1H-imidazol-1-yl)phenylamino)-2-phenyl-2,3-dihydropyrido[3,2-f][1,4]oxazepin-4(5H)-yl)acetonitrile;
4-(2-fluoroethyl)-N-(3-methoxy-4-(4-methyl-1H-imidazol-1-yl)phenyl)-2-phenyl-2,3,4,5-tetrahydropyrido[3,2-f][1,4]oxazepin-8-amine;
N-(3-fluoro-4-(4-methyl-1H-imidazol-1-yl)phenyl)-4-methyl-2-phenyl-2,3,4,5-tetrahydropyrido[3,2-f][1,4]oxazepin-8-amine;
1-(8-(3-methoxy-4-(4-methyl-1H-imidazol-1-yl)phenylamino)-2-phenyl-2,3-dihydropyrido[3,2-f][1,4]oxazepin-4(5H)-yl)ethanone; and
N-(4-(1H-imidazol-1-yl)phenyl)-4-methyl-2-phenyl-2,3,4,5-tetrahydropyrido[3,2-f][1,4]oxazepin-8-amine.

In a second aspect, the present invention provides a pharmaceutical composition comprising an effective amount of a compound according to the first aspect of the invention, or a pharmaceutically acceptable salt thereof, in association with a pharmaceutically acceptable excipient, carrier or diluent.

In a third aspect, the present invention provides a compound according to the first aspect of the invention, of a pharmaceutically acceptable salt thereof, for use as a medicament.

In one embodiment of the third aspect, the present invention provides a compound according to the first aspect, or a pharmaceutically acceptable salt thereof, for use in treating or preventing Aβ-related pathology.

In another embodiment of the third aspect, the present invention provides a compound according to the first aspect, or a pharmaceutically acceptable salt thereof, for use in treating or preventing Aβ-related pathologies selected from the group of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In a fourth aspect, the present invention provides use of a compound according to the first aspect, or a pharmaceutically acceptable salt thereof, for manufacture of a medicament for treating or preventing an Aβ-related pathology.

In one embodiment of the fourth aspect, said Aβ-related pathologies are selected from the group of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In a fifth aspect, the present invention provides a method of treating or preventing an Aβ-related pathology in a mammal, comprising administering to said mammal a therapeutically effective amount of a compound according to the first aspect of the present invention, or a pharmaceutically acceptable salt thereof.

In one embodiment of said fifth aspect, a method of treating or preventing an Aβ-related pathology in a mammal is provided, said method comprising administering to said mammal a therapeutically effective amount of a compound according to said first aspect, or a pharmaceutically acceptable salt thereof, and at least one cognitive enhancing agent, memory enhancing agent, acetyl choline esterase inhibitor, anti-inflammatory agents or atypical antipsychotic agents.

In another embodiment of said fifth aspect, said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

### DEFINITIONS

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

The term “alkoxy”, unless stated otherwise, refers to radicals of the general formula —O—R, wherein R is selected from a hydrocarbon radical. For example “C₁₋₆ alkoxy” denotes alkoxy having 1, 2, 3, 4, 5 or 6 carbon atoms. Examples of alkoxy include, but are not limited to, methoxy, ethoxy, propoxy, isopropoxy, butoxy, t-butoxy and isobutoxy.

As used herein, “alkenyl”, used alone or as a suffix or prefix, is intended to include both branched and straight chain aliphatic hydrocarbon groups comprising at least one carbon-carbon double bond (—C═C—) and having from 2 to 12 carbon atoms or if a specified number of carbon atoms is provided then that specific number would be intended. For example “C₂₋₆ alkenyl” denotes alkenyl having 2, 3, 4, 5 or 6 carbon atoms.

As used herein, “alkynyl”, used alone or as a suffix or prefix, is intended to include both branched and straight chain aliphatic hydrocarbon groups comprising at least one carbon-carbon triple bond (—C≡C—) and having from 2 to 12 carbon atoms or if a specified number of carbon atoms is provided then that specific number would be intended. For example “C₂₋₆ alkynyl” denotes alkynyl having 2, 3, 4, 5 or 6 carbon atoms.

As used herein, “carbocyclyl”, used alone or as suffix or prefix, is intended to include cyclic non-aromatic hydrocarbon groups from 3 to 14 ring carbon atoms or if a specified number of carbon atoms is provided then that specific number would be intended, wherein a —CH₂— group can optionally be replaced by a —C(O)—. In one embodiment, “carbocyclyl” is a monocyclic ring containing 5 or 6 atoms or a bicyclic ring containing 9 or 10 atoms. Examples of carbocyclyls include cyclopropyl, cyclobutyl, cyclopentyl, cyclohexyl, cycloheptyl 1-oxocyclopentyl, cyclopentyl, cyclopentenyl, cyclohexyl, cyclohexenyl, phenyl, naphthyl, tetralinyl, indanyl or 1-oxoindanyl. According to one embodiment, the cycloalkyl group can comprise cycloalkyl groups that are substituted with other rings including fused ring. Example of cycloalkyl groups that are substituted with fused rings include, but are not limited to, adamantly, bornyl, camphenyl, bicycle[2.2.2]octyl, tetrahydronaphthyl and indanyl groups.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic and, for example, the other cyclic rings can be cycloalkyls, cycloalkenyls, cycloalkynyls, aryls and/or heterocyclyls.

As used herein, “halo” or “halogen” or “halogenated” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, imidazothiazolyl and the like. In some embodiments, the heteroaryl group has from 1 to about 16 carbon atoms, and in further embodiments from about 3 to about 16 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

A “heterocyclyl” is a saturated or partially unsaturated monocyclic ring containing 4-7 atoms of which at least one atom is chosen from nitrogen, sulphur or oxygen, which may, unless otherwise specified, be carbon or nitrogen linked, wherein a —CH₂— group can optionally be replaced by a —C(O)—, or a ring nitrogen and/or sulphur atom may be optionally oxidised to form the N-oxide and or the S-oxides. In one aspect of the present invention, “heterocyclyl” is a saturated monocyclic ring containing 4 or 5 or 6 atoms. Examples of heterocyclic groups include without limitation azetidyl, morpholinyl, piperidyl, tetrahydropyranyl, 1,4-dioxanyl, 1,3-dioxolanyl, 1,2-oxathiolanyl, piperazinyl, pyrrolidinyl, tetrahydrofuranyl and thiomorpholino.

As used herein, the phrase “protecting group” means temporary substituents protecting a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been extensively reviewed (e.g. Jarowicki, K.; Kocienski, P. Perkin Trans. 1, 2001, issue 18, p. 2109).

### DETAILED DESCRIPTION OF THE INVENTION

In a first aspect, the invention provides a compound of Formula (I) or (II)

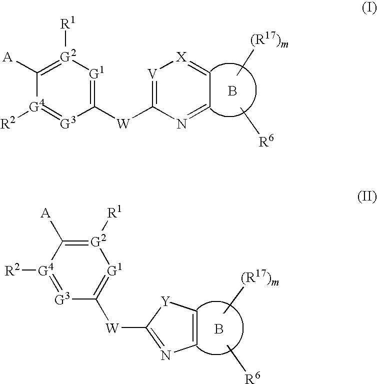

wherein
G¹, G², G³ and G⁴ are independently selected from nitrogen and carbon;
A is a 5 to 6 membered heteroaryl ring, wherein at least one of the ring forming atoms is selected from nitrogen and the remaining ring forming atoms are selected from carbon, nitrogen, sulphur and oxygen, wherein said heteroaryl is optionally substituted with one or more substituents selected from halogen, cyano, nitro, C₁₋₆alkyl, C₃₋₆cycloalkyl, C₂₋₆alkenyl, C₂₋₆alkynyl, SR⁴, N(R⁴)R⁵ and OR⁴, and wherein said C₁₋₆alkyl, C₂₋₆alkenyl or C₂₋₆alkynyl is optionally substituted with halogen, hydroxy, cyano, C₁₋₆alkoxy and OC₃₋₆cycloalkyl;
R¹ and R² are independently selected from hydrogen, halogen, hydroxy, cyano, nitro, C₁₋₆alkyl, C₂₋₆alkenyl, C₂₋₆alkynyl, C₃₋₆carbocyclyl, C₁₋₆alkoxy, OC₃₋₆carbocyclyl, N(R⁴)R⁵, N(R⁴)C(O)R⁵, N(R⁴)S(O)₂R⁵, C(O)R⁴, C(O)N(R⁴)R⁵, SR⁴, S(O)R⁴, S(O)₂R⁴ wherein said C₁₋₆alkyl, C₂₋₆alkenyl, C₂₋₆alkynyl, C₃₋₆carbocyclyl, C₁₋₆alkoxy or OC₃₋₆carbocyclyl is optionally substituted with one to three substituents selected from halogens, cyano, hydroxy, C₁₋₆alkoxy and OC₃₋₆carbocyclyl;
R¹ and said A ring may together form a ring;
X and V are independently selected from nitrogen or CR³;
R³ is selected from hydrogen, halogen, cyano, C₁₋₆alkyl, C₁₋₆alkoxy, OC₃₋₆carbocyclyl wherein said C₁₋₆alkyl, C₁₋₆alkoxy and OC₃₋₆carbocyclyl is optionally substituted with one or more halogen, cyano, hydroxy, SR⁴, S(O)R⁴, S(O)₂R⁴, N(R⁴)R⁵, N(R⁴)C(O)R⁵, C₁₋₆alkoxy, OCF₃, OCF₂H and OCFH₂;
R⁴ and R⁵ are independently selected from hydrogen, C₁₋₆alkyl wherein said C₁₋₆alkyl is optionally substituted with one or more substituents selected from halogen, OCF₃, OCF₂H and OCFH₂;
R⁴ and R⁵ may together form a 5 to 7 membered heterocycle ring containing one or more heteroatomes selected from N, O or S, wherein if said heterocyclic ring contains a nitrogen, that nitrogen may be optionally substituted by a group of substituents selected from C₁₋₆alkyl, C(O)C₁₋₆alkyl;
Y is selected from —N(R⁷)—, —S— and —O—;

### W is —N(R⁷)—, —C(R¹⁰)(R¹¹)— or —O—;

B is a saturated or partially unsaturated 5 to 7 membered ring, wherein two of the ring forming atoms are independently selected from nitrogen, sulfur and oxygen and the other ring forming atoms are carbon, and wherein a —CH₂— group can be optionally replaced by —C(O)— or —C(carbocyclyl)-;
R⁶ is selected from C₁₋₁₀alkyl, aryl, heteroaryl, C₁₋₆alkylaryl, C₁₋₆alkylheteroaryl, C₄₋₁₀heterocyclyl, C₃₋₁₀carbocyclyl; C₁₋₄alkylC₁₋₆heterocyclyl or C₁₋₄alkylC₃₋₁₀carbocyclyl wherein said C₁₋₁₀alkyl, aryl, heteroaryl, C₁₋₆alkylaryl, C₁₋₆alkylheteroaryl, C₄₋₁₀heterocyclyl, C₃₋₁₀carbocyclyl; C₁₋₄alkylC₁₋₆heterocyclyl or C₁₋₄alkylC₃₋₁₀-carbocyclyl is optionally substituted with one or more substituents selected from halogen, cyano, hydroxy, nitro, C₁₋₆alkyl, C₂₋₆alkenyl, C₂₋₆alkynyl, C₁₋₆alkoxy, OC₃₋₆carbocyclyl, C₃₋₁₀carbocyclyl, heterocyclyl, SR⁷, S(O)R⁷, S(O)₂R⁷, S(O)₂N(R⁷)R⁸, C(O)R⁷, C(O)N(R⁷)R⁸, N(R⁷)R⁸, N(R⁹)C(O)N(R⁷)R⁸, N(R⁹)S(O)₂R⁷ and N(R⁷)C(O)R⁸; and wherein said C₁₋₆alkyl, C₂₋₆alkenyl, C₂₋₆alkynyl, C₁₋₆alkoxy, OC₃₋₆carbocyclyl, C₃₋₁₀carbocyclyl or heterocyclyl is optionally substituted with one or more substituents selected from cyano, hydroxy, halogen, OR⁷, S(O)₂R⁷, C(O)R⁷, C(O)N(R⁷)R⁸, N(R⁷)R⁸ and N(R⁹)C(O)N(R⁷)R⁸;
R⁷, R⁸ and R⁹ are independently selected from hydrogen, C₄₋₈heterocyclyl and C₃₋₁₀carbocyclyl; C₁₋₁₀alkyl, C₁₋₆alkylOC₁₋₆alkyl, aryl or heteroaryl; wherein said C₄₋₈heterocyclyl, C₃₋₁₀carbocyclyl; C₃₋₁₀alkyl, C₁₋₆alkylOC₁₋₆alkyl, aryl and heteroaryl is optionally substituted with one or more substituents selected from halogen, cyano, hydroxy, OCF₃, OCF₂H, OCFH₂, C₁₋₆alkoxy, OC₃₋₆carbocyclyl and OC₄₋₆heterocyclyl;
R⁷ and R⁸ may together form a 5 to 7 membered heterocycle ring containing one or more heteroatomes selected from N, O or S, wherein if said heterocyclic ring contains an —NH— moiety that nitrogen may be optionally substituted by a group of substituents selected from C₄₋₈heterocyclyl and C₃₋₆carbocyclyl; C₁₋₆alkyl, C₁₋₁₀alkylOC₁₋₁₀alkyl, aryl, C(O)C₁₋₁₀alkyl or heteroaryl;
R¹⁰ and R¹¹ are independently selected from hydrogen, halogen, C₁₋₆alkyl, C₁₋₆alkoxy and C₁₋₆alkylOC₁₋₆alkyl;

R¹⁷ is independently selected from hydrogen, hydroxy, halogen, cyano, C₁₋₆alkyl, C₂₋₆alkenyl, C₂₋₆alkynyl, aryl, heteroaryl, C₁₋₆alkylaryl, C₁₋₆alkylheteroaryl, C₃₋₇-carbocyclyl, heterocyclyl, OR⁷, SR⁷, S(O)R⁷, S(O)₂R⁷, S(O)₂N(R⁷)R⁸, N(R⁹)S(O)₂R⁷, N(R⁹)S(O)₂N(R⁷)R⁸, N(R⁷)R⁸, N(R⁷)C(O)R⁸, N(R⁹)C(O)N(R⁷)R⁸, C(O)R⁷ and C(O)N(R⁷)R⁸ wherein said C₁₋₆alkyl, C₂₋₆alkenyl, C₂₋₆alkynyl, aryl, heteroaryl, C₁₋₆alkylaryl, C₁₋₆alkylheteroaryl, C₃₋₇carbocyclyl, and heterocyclyl optionally is substituted with one or more substituents selected from cyano, hydroxy, halogen, OR⁷, S(O)₂R⁷, S(O)₂N(R⁷)R⁸, C(O)R⁷, C(O)N(R⁷)R⁸, N(R⁷)R⁸, N(R⁹)S(O)₂N(R⁷)R⁸ and N(R⁹)C(O)N(R⁷)R⁸; and

m is 2, 3 or 4;
or a pharmaceutically acceptable salt thereof
with the proviso that the compounds 8-(4-(1H-tetrazol-1-yl)phenoxy)-1,3,7-trimethyl-1H-purine-2,6(3H,7H)-dione and 8-(4-(1H-tetrazol-1-yl)phenoxy)-7-allyl-1,3-dimethyl-1H-purine-2,6(3H,7H)-dione are excluded.

In one embodiment of said first aspect of the invention, the compound is of Formula (III), or a pharmaceutically acceptable salt thereof:

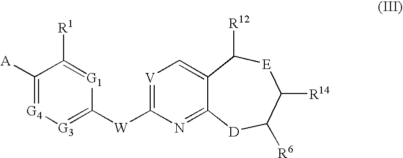

wherein
G₁, G₃, G₄ and V are independently selected from carbon and nitrogen;
A, R¹ and R⁶ have the same meaning as previously defined;
W is selected from —NH— and —O—;
D is selected from —N(R¹⁶)— and —O—;
E is selected from —N(R¹³)—, —S(O)—, —S(O)₂— and —O—;
R¹³ when present is selected from hydrogen, C₁₋₆alkyl, C₁₋₆alkylOC₁₋₆alkyl, C₂₋₆alkenyl, C₂₋₆alkynyl, carbocyclyl, heterocyclyl, C(O)R⁷, C(O)N(R⁷)(R⁸), C(O)OR⁷, S(O)₂R⁷ and S(O)₂N(R⁷)(R⁸), wherein said C₁₋₆alkyl, C₁₋₆alkylOC₁₋₆alkyl, C₂₋₆alkenyl, C₂₋₆alkynyl, carbocyclyl or heterocyclyl is optionally substituted with one or more substituents selected from halogen, hydroxy, cyano, C(O)OR⁷, N(R⁸)(R⁸), C₁₋₆alkyl, C₁₋₆alkoxy, heterocyclyl and carbocyclyl and wherein R⁷ and R⁸ have the same meaning as defined above;
R¹⁶ when present, is selected from C₁₋₁₀alkyl, C₂₋₆alkenyl, C₂₋₆alkynyl, C₁₋₁₀carbocyclyl, heterocyclyl wherein said C₁₋₁₀alkyl, C₂₋₆alkenyl, C₂₋₆alkynyl, C₁₋₁₀carbocyclyl, heterocyclyl is optionally substituted with one or more substituents selected from cyano, hydroxy, and halogen; and
R¹² and R¹⁴ are independently selected from, hydrogen, ═O, cyano, OR⁷, SR⁷, S(O)R⁷, S(O)₂R⁷, S(O)₂N(R⁷)R⁸, N(R⁹)S(O)₂R⁷, N(R⁹)S(O)₂N(R⁷)R⁸, N(R⁷)R⁸, N(R⁷)C(O)R⁸, N(R⁹)C(O)N(R⁷)R⁸, C(O)R⁷, C(O)N(R⁷)R⁸, C₁₋₆alkyl, C₂₋₆alkenyl, C₂₋₆alkynyl, C₁₋₆alkoxy, aryl, heteroaryl, C₁₋₆alkylaryl, C₁₋₆alkylheteroaryl, C₄₋₁₀heterocyclyl, C₃₋₁₀carbocyclyl; C₁₋₄alkC₁₋₆heterocyclyl or C₁₋₄alkC₃₋₁₀carbocyclyl wherein said C₁₋₆alkyl, aryl, heteroaryl, C₁₋₆alkylaryl, C₁₋₆alkylheteroaryl, C₄₋₁₀heterocyclyl, C₃₋₁₀ carbocyclyl; C₁₋₄alkC₁₋₆heterocyclyl or C₁₋₄alkC₃₋₁₀carbocyclyl is optionally substituted with one or more substituents selected from halogen, cyano, hydroxy, nitro, C₁₋₆alkyl, C₂₋₆alkenyl, C₂₋₆alkynyl, C₁₋₆alkoxy, OC₃₋₆carbocyclyl, C₃₋₁₀carbocyclyl, heterocyclyl, SR⁷, S(O)R⁷, S(O)₂R⁷, S(O)₂N(R⁷)R⁸, C(O)R⁷, C(O)N(R⁷)R⁸, N(R⁷)R⁸, N(R⁹)C(O)N(R⁷)R⁸, N(R⁹)S(O)₂R⁷ and N(R⁷)C(O)R⁸; and wherein said C₁₋₆alkyl, C₂₋₆alkenyl, C₂₋₆alkynyl, C₁₋₆alkoxy, OC₃₋₆carbocyclyl, C₃₋₁₀carbocyclyl or heterocyclyl is optionally substituted with one or more substituents selected from cyano, hydroxy, halogen, OR⁷, S(O)₂R⁷, C(O)R⁷, C(O)N(R⁷)R⁸, N(R⁷)R⁸ and N(R⁹)C(O)N(R⁷)R⁸, wherein R⁷, R⁸ and R⁹ have the same meaning as previously defined.

In another embodiment of the first aspect of the invention is provided a compound of Formula (III), or a pharmaceutically acceptable salt thereof, wherein:

G₁, G₃, G₄, V, W, D and E has the same meaning as for the first embodiment of the first aspect of the invention;
A is a five-membered heteroaryl ring, wherein at least one of the ring forming atoms is selected from nitrogen and the remaining ring forming atoms are selected from carbon, nitrogen, and oxygen, wherein said heteroaryl is optionally substituted with one or more substituents selected from halogen, cyano, nitro, C₁₋₆alkyl, and OR⁷, wherein R⁴ has the same meaning as previously defined;
R¹ is selected from hydrogen, halogen, hydroxy, cyano, nitro, C₁₋₆alkyl, and C₁₋₆alkoxy;
R⁶ is selected from C₁₋₁₀alkyl, aryl, heteroaryl, C₁₋₆alkylaryl, C₄₋₁₀heterocyclyl, C₃₋₁₀carbocyclyl, C₁₋₄alkC₁₋₆heterocyclyl or C₁₋₄alkC₃₋₁₀carbocyclyl wherein said C₁₋₁₀alkyl, aryl, heteroaryl, C₁₋₆alkylaryl, C₄₋₁₀heterocyclyl, C₃₋₁₀carbocyclyl; C₁₋₄alkC₁₋₆heterocyclyl or C₁₋₄alkC₃₋₁₀carbocyclyl is optionally substituted with one or more substituents selected from halogen, cyano, hydroxy, nitro, C₁₋₆alkoxy and C₁₋₆alkyl;
R¹² is selected from hydrogen and ═O;
R¹³, if present, is selected from hydrogen, C₁₋₆alkyl and —C(O)R⁷, wherein said C₁₋₆alkyl is optionally substituted with one or more substituents selected from halogen, cyano, hydroxy and C₁₋₆alkoxy, and wherein R⁷ has the same meaning as previously defined;
R¹⁴ is selected from hydrogen, ═O, hydroxy, halogen and C₁₋₆alkyl, wherein said C₁₋₆alkyl optionally is substituted with one or more substituents selected from halogen, cyano, hydroxy and C₁₋₆alkoxy; and
R¹⁶ when present, is selected from substituents selected from hydrogen, C₁₋₁₀alkyl, C₃₋₁₀carbocyclyl wherein said C₁₋₁₀alkyl and C₃₋₁₀carbocyclyl is optionally substituted with one or more substituents selected from cyano, hydroxy, and halogen.

In another embodiment of the first aspect of the invention is provided a compound of Formula (III), or a pharmaceutically acceptable salt thereof, wherein:

G₁, G₃, G₄, V, W, D, E and R¹², has the same meaning as defined in relation to the second embodiment of the first aspect of the invention;
A is selected from imidazole, oxazole and oxadiazole, wherein said imidazole, oxazole and oxadiazole optionally is substituted with C₁₋₆alkyl, and preferably substituted with methyl;
R¹ is selected from hydrogen, halogen and C₁₋₆alkoxy;
R¹³, if present, is selected from C₁₋₆alkyl, —C(O)C₁₋₆alkyl and C₁₋₆hydroxyalkyl, wherein said C₁₋₆alkyl optionally is substituted with halogen;
R¹⁴ is selected from hydrogen and ═O;
R⁶ is selected from phenyl, benzyl, tetrahydrofuranyl and cyclopentyl, wherein said phenyl, benzyl, tetrahydrofuranyl and cyclopentyl optionally are substituted with one or more substituents selected from halogen, hydroxy, C₁₋₆alkoxy and cyano; and
R¹⁶, if present, is selected from hydrogen, methyl, and cyclopentyl.

In another embodiment of the first aspect of the invention is provided a compound of Formula (III), or a pharmaceutically acceptable salt thereof, wherein R¹⁴ is hydrogen.

In another embodiment of the first aspect of the invention is provided a compound of Formula (III), or a pharmaceutically acceptable salt thereof, wherein D is —O—.

In another embodiment of the first aspect of the invention is provided a compound of Formula (III), or a pharmaceutically acceptable salt thereof, wherein E is —N(R¹³)—.

In another embodiment of the first aspect of the invention is provided a compound of Formula (III), or a pharmaceutically acceptable salt thereof, wherein

### W is —NH—.

In another embodiment of the first aspect of the invention is provided a compound of Formula (III), or a pharmaceutically acceptable salt thereof, wherein V is —CH═.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—; and

### W is —NH—.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—; and

### V is —CH═.

In another embodiment of the first aspect of the invention is provided a compound of Formula (III), or a pharmaceutically acceptable salt thereof, wherein:

one of G₁, G₃ or G₄ is —N═ and the other two are —CH═; or each of G₁, G₃ and G₄ is —CH═.

In another embodiment of the first aspect of the invention is provided a compound of Formula (III), or a pharmaceutically acceptable salt thereof, wherein

one of G₁, G₃ or G₄ is —N═ and the other two are —CH═.

In another embodiment of the first aspect of the invention is provided a compound of Formula (III), or a pharmaceutically acceptable salt thereof, wherein

G₁ is —N═ and each of G₃ and G₄ is —CH═.

In another embodiment of the first aspect of the invention is provided a compound of Formula (III), or a pharmaceutically acceptable salt thereof, wherein

each of G₁, G₃ and G₄ is —CH═.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═; and

One of G₁, G₃ or G₄ is —N═ and the other two are —CH═; or each of G₁, G₃ and G₄ is —CH═.

In another embodiment of the first aspect of the invention is provided a compound of Formula (III), or a pharmaceutically acceptable salt thereof, wherein

R¹ is hydrogen, C₁₋₂alkoxy or halo.

In another embodiment of the first aspect of the invention is provided a compound of Formula (III), or a pharmaceutically acceptable salt thereof, wherein

R¹ is hydrogen or C₁₋₂alkoxy.

In another embodiment of the first aspect of the invention is provided a compound of Formula (III), or a pharmaceutically acceptable salt thereof, wherein

R¹ is C₁₋₂alkoxy.

In another embodiment of the first aspect of the invention is provided a compound of Formula (III), or a pharmaceutically acceptable salt thereof, wherein

R¹ is methoxy.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═;

one of G₁, G₃ or G₄ is —N═ and the other two are —CH═; or each of G₁, G₃ and G₄ is —CH═;
and
R¹ is hydrogen, C₁₋₂alkoxy or halo.

In another embodiment of the first aspect of the invention is provided a compound of Formula (III), or a pharmaceutically acceptable salt thereof, wherein

R¹² is hydrogen or ═O.

In another embodiment of the first aspect of the invention is provided a compound of Formula (III), or a pharmaceutically acceptable salt thereof, wherein

R¹² is hydrogen.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═;

One of G₁, G₃ or G₄ is —N═ and the other two are —CH═; or each of G₁, G₃ and G₄ is —CH═;
R¹ is hydrogen, C₁₋₂alkoxy or halo; and
R¹² is hydrogen or ═O.

In another embodiment of the first aspect of the invention is provided a compound of Formula (III), or a pharmaceutically acceptable salt thereof, wherein R¹³ is hydrogen; C₁-C₃ alkyl optionally substituted with hydroxy, cyano or halo; C₃-C₆ carbocyclyl, C₁-C₂ alkylcarbonyl-; or (C₁-C₂alkyl)₂NC(O)—.

In another embodiment of the first aspect of the invention is provided a compound of Formula (III), or a pharmaceutically acceptable salt thereof, wherein R¹³ is C₁-C₃ alkyl.

In another embodiment of the first aspect of the invention is provided a compound of Formula (III), or a pharmaceutically acceptable salt thereof, wherein R¹³ is methyl or hydrogen.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═;

one of G₁, G₃ or G₄ is —N═ and the other two are —CH═; or each of G₁, G₃ and G₄ is —CH═;
R¹ is hydrogen, C₁₋₂alkoxy or halo;
R¹² is hydrogen or ═O; and
R¹³ is hydrogen; C₁-C₃ alkyl optionally substituted with hydroxy, cyano or halo; C₃-C₆ carbocyclyl, C₁-C₂ alkylcarbonyl-; or (C₁-C₂alkyl)₂NC(O)—.

In another embodiment of the first aspect of the invention is provided a compound of Formula (III), or a pharmaceutically acceptable salt thereof, wherein: A is triazolyl, imidazolyl, pyrazolyl, oxadiazolyl or oxazolyl, optionally substituted with C₁₋₂alkyl.

In another embodiment of the first aspect of the invention is provided a compound of Formula (III), or a pharmaceutically acceptable salt thereof, wherein: A is triazolyl, imidazolyl or pyrazolyl, optionally substituted with C₁₋₂alkyl.

In another embodiment of the first aspect of the invention is provided a compound of Formula (III), or a pharmaceutically acceptable salt thereof, wherein: A is triazolyl or pyrazolyl, optionally substituted with C₁₋₂alkyl.

In another embodiment of the first aspect of the invention is provided a compound of Formula (III), or a pharmaceutically acceptable salt thereof, wherein: A is triazolyl optionally substituted with C₁₋₂alkyl.

In another embodiment of the first aspect of the invention is provided a compound of Formula (III), or a pharmaceutically acceptable salt thereof, wherein: A is triazolyl optionally substituted with methyl.

In another embodiment of the first aspect of the invention is provided a compound of Formula (III), or a pharmaceutically acceptable salt thereof, wherein: A is pyrazolyl optionally substituted with C₁₋₂alkyl.

In another embodiment of the first aspect of the invention is provided a compound of Formula (III), or a pharmaceutically acceptable salt thereof, wherein: A is pyrazolyl optionally substituted with methyl.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═;

one of G₁, G₃ or G₄ is —N═ and the other two are —CH═; or each of G₁, G₃ and G₄ is —CH═;
R¹ is hydrogen, C₁₋₂alkoxy or halo;
R¹² is hydrogen or ═O;
R¹³ is hydrogen; C₁-C₃ alkyl optionally substituted with hydroxy, cyano or halo; C₃-C₆ carbocyclyl, C₁-C₂ alkylcarbonyl-; or (C₁-C₂alkyl)₂NC(O)—; and
A is triazolyl, imidazolyl, pyrazolyl, oxadiazolyl or oxazolyl, optionally substituted with C₁₋₂alkyl.

In another embodiment of the first aspect of the invention is provided a compound of Formula (III), or a pharmaceutically acceptable salt thereof, wherein: R⁶ is hydrogen; phenyl; pyridinyl optionally substituted with C₁-C₂ alkyl; C₃-C₅ carbocyclyl; pyrazolyl optionally substituted with one or two C₁-C₂ alkyl groups; or C₂-C₄ alkoxyC₁-C₂ alkyl-;

In another embodiment of the first aspect of the invention is provided a compound of Formula (III), or a pharmaceutically acceptable salt thereof, wherein R⁶ is phenyl.

In another embodiment of the first aspect of the invention is provided a compound of Formula (III), or a pharmaceutically acceptable salt thereof, wherein:

R¹⁴ is hydrogen;

### D is —O—;

### E is —N(R¹³)—;

### W is —NH—;

### V is —CH═;

one of G₁, G₃ or G₄ is —N═ and the other two are —CH═; or each of G₁, G₃ and G₄ is —CH═;
R¹ is hydrogen, C₁₋₂alkoxy or halo;
R¹² is hydrogen or ═O;
R¹³ is hydrogen, C₁-C₃ alkyl optionally substituted with hydroxy, cyano or halo; C₃-C₆ carbocyclyl, C₁-C₂ alkylcarbonyl-, or (C₁-C₂alkyl)₂NC(O)—;
A is triazolyl, imidazolyl, pyrazolyl, oxadiazolyl or oxazolyl, optionally substituted with C₁₋₂alkyl; and
R⁶ is phenyl; pyridinyl optionally substituted with a C₁-C₂ alkyl group; pyrazolyl optionally substituted with one or two C₁-C₂ alkyl groups; C₃-C₅ carbocyclyl; or C₂-C₄alkoxyC₁-C₂alkyl.

In another embodiment of the first aspect of the invention is provided a compound of Formula (III), or a pharmaceutically acceptable salt thereof, wherein:

### W is —NH—;

### D is —O—;

### E is —N(R¹³)—;

R¹⁴ is hydrogen;
A is selected from imidazole, pyrazole, triazole, oxazole and oxadiazole, wherein said imidazole, pyrazole, triazole, oxazole and oxadiazole optionally is substituted with C₁₋₆alkyl, and optionally substituted with methyl;
R¹ is selected from hydrogen, halogen and C₁₋₆alkoxy;
R¹³, if present, is selected from hydrogen, C₁₋₃alkyl, —C(O)C₁₋₃alkyl and C₁₋₃hydroxyalkyl, wherein said C₁₋₆alkyl optionally is substituted with halogen;
R⁶ is selected from phenyl, cyclopropyl, pyridyl, isopropoxymethyl and pyrazolyl, wherein said phenyl, cyclopropyl, pyridyl, isopropoxymethyl, pyrazolyl optionally are substituted with one or more substituents selected from methyl or halogen.

In another embodiment of the first aspect of the invention is provided a compound of Formula (III), or a pharmaceutically acceptable salt thereof, wherein:

A is pyrazole or triazole, optionally substituted with methyl;
R¹ is methoxy;
R⁶ is phenyl;

### W is —NH—;

### D is —O—;

### E is —N(R¹³)—;

Each of G₁ G₃ and G₄ is —CH═, or G₁ is —N═ and both G₃ and G₄ are —CH═;

R¹² is hydrogen or ═O;
R¹³ is methyl;
R¹⁴ is hydrogen; and

### V is —CH═.

In another embodiment of the first aspect of the invention is provided a compound of Formula (IV), or a pharmaceutically acceptable salt thereof, wherein:

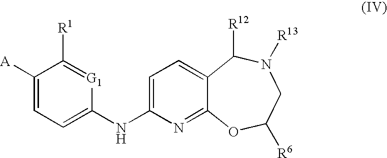

A is imidazolyl, pyrazolyl or triazoyl, optionally substituted with C₁-C₂ alkyl;

### G₁ is —N═ or —CH═;

R¹ is hydrogen, C₁-C₂alkoxy, or halo;
R⁶ is hydrogen; phenyl; pyridinyl optionally substituted with C₁-C₂ alkyl; C₃-C₅ carbocyclyl; pyrazolyl optionally substituted with one or two C₁-C₂ alkyl groups; C₁-C₄ alkoxyC₁-C₄ alkyl-;
R¹² is hydrogen or ═O; and
R¹³ is hydrogen; C₁-C₃ alkyl optionally substituted with hydroxy, cyano or halo; C₃-C₅ carbocyclyl, C₁-C₂ alkylcarbonyl-; or (C₁-C₂alkyl)₂NC(O)—.

In another embodiment of the first aspect of the invention is provided a compound of Formula (IV), or a pharmaceutically acceptable salt thereof, wherein:

A is pyrazolyl or triazoyl, optionally substituted with C₁-C₂ alkyl;

### G₁ is —N═ or —CH═;

R¹ is hydrogen or C₁-C₂ alkoxy;
R⁶ is hydrogen; phenyl; pyridinyl optionally substituted with C₁-C₂ alkyl; C₃-C₅ carbocyclyl; pyrazolyl optionally substituted with one or two C₁-C₂ alkyl groups; C₁-C₄ alkoxyC₁-C₄ alkyl-;
R¹² is hydrogen; and
R¹³ is hydrogen; C₁-C₃ alkyl optionally substituted with hydroxy, cyano or halo; C₃-C₅ carbocyclyl, C₁-C₂ alkylcarbonyl-; or (C₁-C₂alkyl)₂NC(O)—.

In another embodiment of the first aspect of the invention is provided a compound of Formula (IV), or a pharmaceutically acceptable salt thereof, wherein:

A is pyrazolyl or triazoyl, optionally substituted with C₁-C₂ alkyl;

### G₁ is —N═ or —CH═;

R¹ is hydrogen or C₁-C₂alkoxy;
R⁶ is phenyl;
R¹² is hydrogen; and
R¹³ is C₁-C₂ alkyl.

In another embodiment of the first aspect of the invention is provided a compound of Formula (III) or (IV), or a pharmaceutically acceptable salt thereof,

selected from
N-[3-methoxy-4-(4-methyl-1H-imidazol-1-yl)phenyl]-4-methyl-2-phenyl-2,3,4,5-tetrahydropyrido[3,2-f][1,4]oxazepin-8-amine;
4-methyl-N-[4-(2-methyl-1H-imidazol-1-yl)phenyl]-2-phenyl-2,3,4,5-tetrahydropyrido[3,2-f][1,4]oxazepin-8-amine;
4-(2-hydroxyethyl)-8-(3-methoxy-4-(4-methyl-1H-imidazol-1-yl)phenylamino)-2-phenyl-3,4-dihydropyrido[3,2-f][1,4]oxazepin-5(2H)-one;
8-{[3-methoxy-4-(4-methyl-1H-imidazol-1-yl)phenyl]amino}-4-methyl-2-phenyl-3,4-dihydropyrido[3,2-f][1,4]oxazepin-5(2H)-one;
4-methyl-8-{[4-(1,3-oxazol-5-yl)phenyl]amino}-2-phenyl-3,4-dihydropyrido[3,2-f][1,4]oxazepin-5(2H)-one;
4-methyl-N-(6-(2-methyl-1H-imidazol-1-yl)pyridin-3-yl)-2-phenyl-2,3,4,5-tetrahydropyrido[3,2-f][1,4]oxazepin-8-amine;
6,9-dimethyl-2-(4-(oxazol-5-yl)phenoxy)-8-phenyl-6,7,8,9-tetrahydro-5H-pyrimido[4,5-e][1,4]diazepin-5-one;
8-{[4-(1H-imidazol-1-yl)phenyl]amino}-4-methyl-2-phenyl-3,4-dihydropyrido[3,2-f][1,4]oxazepin-5(2H)-one;
4-methyl-8-{[4-(2-methyl-1H-imidazol-1-yl)phenyl]amino}-2-phenyl-3,4-dihydropyrido[3,2-f][1,4]oxazepin-5(2H)-one;
4-methyl-8-{[4-(3-methyl-1,2,4-oxadiazol-5-yl)phenyl]amino}-2-phenyl-3,4-dihydropyrido[3,2-f][1,4]oxazepin-5(2H)-one;
1-cyclopentyl-4-methyl-8-(4-(2-methyl-1H-imidazol-1-yl)phenylamino)-3,4-dihydro-1H-pyrido[2,3-e][1,4]diazepin-5(2H)-one;
2-(4-(1H-imidazol-1-yl)phenoxy)-6-methyl-8-phenyl-7,8-dihydropyrimido[5,4-f][1,4]oxazepin-5(6H)-one;
4-methyl-8-{[6-(2-methyl-1H-imidazol-1-yl)pyridin-3-yl]amino}-2-phenyl-3,4-dihydropyrido[3,2-f][1,4]oxazepin-5(2H)-one;
N-(4-(2-methyl-1H-imidazol-1-yl)phenyl)-2-phenyl-3,5-dihydro-2H-[1,4]dioxepino[5,6-b]pyridin-8-amine;
N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
N-(4-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
N-(6-methoxy-5-(4-methyl-1H-imidazol-1-yl)pyridin-2-yl)-2-phenyl-3,5-dihydro-2H-[1,4]dioxepino[5,6-b]pyridin-8-amine;
N-(6-methoxy-5-(4-methyl-1H-imidazol-1-yl)pyridin-2-yl)-4-methyl-2-(tetrahydrofuran-2-yl)-2,3,4,5-tetrahydropyrido[3,2-f][1,4]oxazepin-8-amine;
N-(3-methoxy-4-(4-methyl-1H-imidazol-1-yl)phenyl)-4-methyl-2-(tetrahydrofuran-2-yl)-2,3,4,5-tetrahydropyrido[3,2-f][1,4]oxazepin-8-amine;
4-(difluoromethyl)-N-(4-(2-methyl-1H-imidazol-1-yl)phenyl)-2-phenyl-2,3,4,5-tetrahydropyrido[3,2-f][1,4]oxazepin-8-amine;
1-(8-(4-(2-methyl-1H-imidazol-1-yl)phenylamino)-2-phenyl-2,3-dihydropyrido[3,2-f][1,4]oxazepin-4(5H)-yl)ethanone;
4-methyl-8-(4-(2-methyl-1H-imidazol-1-yl)phenylamino)-2-phenyl-4,5-dihydropyrido[3,2-f][1,4]oxazepin-3(2H)-one;
N-(3-methoxy-4-(4-methyl-1H-imidazol-1-yl)phenyl)-1,4-dimethyl-2-phenyl-2,3,4,5-tetrahydro-1H-pyrido[2,3-e][1,4]diazepin-8-amine;
1,4-dimethyl-8-(4-(2-methyl-1H-imidazol-1-yl)phenylamino)-2-phenyl-4,5-dihydro-1H-pyrido[2,3-e][1,4]diazepin-3(2H)-one;
2-(3-fluorophenyl)-N-(4-methoxy-5-(4-methyl-1H-imidazol-1-yl)pyridin-2-yl)-4-methyl-2,3,4,5-tetrahydropyrido[3,2-f][1,4]oxazepin-8-amine;
N-(3-methoxy-4-(4-methyl-1H-imidazol-1-yl)phenyl)-6-methyl-8-phenyl-5,6,7,8-tetrahydropyrimido[5,4-f][1,4]oxazepin-2-amine;
N-(3-methoxy-4-(4-methyl-1H-imidazol-1-yl)phenyl)-1,4-dimethyl-2-phenyl-2,3,4,5-tetrahydro-1H-pyrido[2,3-e][1,4]diazepin-8-amine;
N-[3-methoxy-4-(4-methyl-1H-imidazol-1-yl)phenyl]-8-phenyl-7,8-dihydro-5H-[1,4]oxathiepino[7,6-d]pyrimidin-2-amine6,6-dioxide;
N-[3-methoxy-4-(4-methyl-1H-imidazol-1-yl)phenyl]-8-phenyl-7,8-dihydro-5H-[1,4]oxathiepino[7,6-d]pyrimidin-2-amine6-oxide;
N-(3-fluoro-4-(4-methyl-1H-imidazol-1-yl)phenyl)-6-methyl-8-phenyl-5,6,7,8-tetrahydropyrimido[5,4-f][1,4]oxazepin-2-amine; and
N-(3-fluoro-4-(4-methyl-1H-imidazol-1-yl)phenyl)-6-(2-methoxyethyl)-8-phenyl-5,6,7,8-tetrahydropyrimido[5,4-f][1,4]oxazepin-2-amine;
   as a free base or a pharmaceutically acceptable salt thereof.

In a further embodiment of the first aspect of the invention is provided a compound according to formula (I), (II) or (III), selected from
4-methyl-8-(4-(3-methyl-1,2,4-oxadiazol-5-yl)phenylamino)-2-phenyl-3,4-dihydropyrido[3,2-f][1,4]oxazepin-5(2H)-one;
4-methyl-8-(4-(2-methyl-1H-imidazol-1-yl)phenylamino)-2-phenyl-3,4-dihydropyrido[3,2-f][1,4]oxazepin-5(2H)-one;
4-methyl-8-(4-(1,3-oxazol-5-yl)phenylamino)-2-phenyl-3,4-dihydropyrido[3,2-f][1,4]oxazepin-5(2H)-one;
8-(3-methoxy-4-(4-methyl-1H-imidazol-1-yl)phenylamino)-4-methyl-2-phenyl-3,4-dihydropyrido[3,2-f][1,4]oxazepin-5(2H)-one;
8-(4-(1H-imidazol-1-yl)phenylamino)-4-methyl-2-phenyl-3,4-dihydropyrido[3,2-f][1,4]oxazepin-5(2H)-one;
4-methyl-8-(6-(2-methyl-1H-imidazol-1-yl)pyridin-3-ylamino)-2-phenyl-3,4-dihydropyrido[3,2-f][1,4]oxazepin-5(2H)-one;
N-(3-methoxy-4-(4-methyl-1H-imidazol-1-yl)phenyl)-4-methyl-2-phenyl-2,3,4,5-tetrahydropyrido[3,2-f][1,4]oxazepin-8-amine;
4-methyl-N-(4-(2-methyl-1H-imidazol-1-yl)phenyl)-2-phenyl-2,3,4,5-tetrahydropyrido[3,2-f][1,4]oxazepin-8-amine;
(R)—N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
(R)—N-(3-methoxy-4-(4-methyl-1H-imidazol-1-yl)phenyl)-4-methyl-2-phenyl-2,3,4,5-tetrahydropyrido[3,2-f][1,4]oxazepin-8-amine;
(R)-1-(8-(6-methoxy-5-(4-methyl-1H-imidazol-1-yl)pyridin-2-ylamino)-2-phenyl-2,3-dihydropyrido[3,2-f][1,4]oxazepin-4(5H)-yl)ethanone;
(R)-6-(2-fluoroethyl)-N-(3-methoxy-4-(4-methyl-1H-imidazol-1-yl)phenyl)-8-phenyl-5,6,7,8-tetrahydropyrimido[5,4-f][1,4]oxazepin-2-amine;
(R)—N-(3-methoxy-4-(4-methyl-1H-imidazol-1-yl)phenyl)-6-methyl-8-phenyl-5,6,7,8-tetrahydropyrimido[5,4-f][1,4]oxazepin-2-amine;
2-(8-(3-methoxy-4-(4-methyl-1H-imidazol-1-yl)phenylamino)-2-phenyl-2,3-dihydropyrido[3,2-f][1,4]oxazepin-4(5H)-yl)acetonitrile;
4-(2-fluoroethyl)-N-(3-methoxy-4-(4-methyl-1H-imidazol-1-yl)phenyl)-2-phenyl-2,3,4,5-tetrahydropyrido[3,2-f][1,4]oxazepin-8-amine;
N-(3-fluoro-4-(4-methyl-1H-imidazol-1-yl)phenyl)-4-methyl-2-phenyl-2,3,4,5-tetrahydropyrido[3,2-f][1,4]oxazepin-8-amine;
1-(8-(3-methoxy-4-(4-methyl-1H-imidazol-1-yl)phenylamino)-2-phenyl-2,3-dihydropyrido[3,2-f][1,4]oxazepin-4(5H)-yl)ethanone;
N-(4-(1H-imidazol-1-yl)phenyl)-4-methyl-2-phenyl-2,3,4,5-tetrahydropyrido[3,2-f][1,4]oxazepin-8-amine;
N-(3-methoxy-4-(4-methyl-1H-imidazol-1-yl)phenyl)-4-methyl-2-(pyridin-3-yl)-2,3,4,5-tetrahydropyrido[3,2-f][1,4]oxazepin-8-amine;
4-(2-hydroxyethyl)-8-(3-methoxy-4-(4-methyl-1H-imidazol-1-yl)phenylamino)-2-phenyl-3,4-dihydropyrido[3,2-f][1,4]oxazepin-5(2H)-one;
(S)—N-(3-methoxy-4-(4-methyl-1H-imidazol-1-yl)phenyl)-4-methyl-2-phenyl-2,3,4,5-tetrahydropyrido[3,2-f][1,4]oxazepin-8-amine;
1-(8-(3-methoxy-4-(4-methyl-1H-imidazol-1-yl)phenylamino)-2-(pyridin-3-yl)-2,3-dihydropyrido[3,2-f][1,4]oxazepin-4(5H)-yl)ethanone;
4-methyl-N-(6-(2-methyl-1H-imidazol-1-yl)pyridin-3-yl)-2-phenyl-2,3,4,5-tetrahydropyrido[3,2-f][1,4]oxazepin-8-amine;
4-(2-hydroxyethyl)-8-(4-(2-methyl-1H-imidazol-1-yl)phenylamino)-2-phenyl-3,4-dihydropyrido[3,2-f][1,4]oxazepin-5(2H)-one;
1-cyclopentyl-4-methyl-8-(4-(2-methyl-1H-imidazol-1-yl)phenylamino)-3,4-dihydro-1H-pyrido[2,3-e][1,4]diazepin-5(2H)-one;
(R)-2-(8-(4-(6,7-dihydro-5H-pyrrolo[2,1-c][1,2,4]triazol-3-yl)phenylamino)-2-phenyl-2,3-dihydropyrido[3,2-f][1,4]oxazepin-4(5H)-yl)ethanol;
2-(2-(1,3-dimethyl-1H-pyrazol-5-yl)-8-(4-(2-methyl-1H-imidazol-1-yl)phenylamino)-2,3-dihydropyrido[3,2-f][1,4]oxazepin-4(5H)-yl)acetonitrile;
2-(4-(1H-imidazol-1-yl)phenoxy)-6-methyl-8-phenyl-7,8-dihydropyrimido[5,4-f][1,4]oxazepin-5(6H)-one;
(R)-4-(4-(2-hydroxyethyl)-2-phenyl-2,3,4,5-tetrahydropyrido[3,2-f][1,4]oxazepin-8-ylamino)-2-methoxybenzonitrile;
(R)-2-(8-(4-(5-methyl-1H-imidazol-1-yl)phenylamino)-2-phenyl-2,3-dihydropyrido[3,2-f][1,4]oxazepin-4(5H)-yl)ethanol;
1-(2-(isopropoxymethyl)-8-(3-methoxy-4-(4-methyl-1H-imidazol-1-yl)phenylamino)-2,3-dihydropyrido[3,2-f][1,4]oxazepin-4(5H)-yl)ethanone;
(R)-2-(8-(3-chloro-4-(1H-1,2,4-triazol-1-yl)phenylamino)-2-phenyl-2,3-dihydropyrido[3,2-f][1,4]oxazepin-4(5H)-yl)ethanol;
2-(2-(1,3-dimethyl-1H-pyrazol-5-yl)-8-(3-methoxy-4-(4-methyl-1H-imidazol-1-yl)phenylamino)-2,3-dihydropyrido[3,2-f][1,4]oxazepin-4(5H)-yl)acetonitrile;
(R)-2-(8-(3-methoxy-4-(5-methyl-1H-imidazol-1-yl)phenylamino)-2-phenyl-2,3-dihydropyrido[3,2-f][1,4]oxazepin-4(5H)-yl)ethanol;
2-(isopropoxymethyl)-N-(3-methoxy-4-(4-methyl-1H-imidazol-1-yl)phenyl)-4-methyl-2,3,4,5-tetrahydropyrido[3,2-f][1,4]oxazepin-8-amine;
2-(8-(3-methoxy-4-(4-methyl-1H-imidazol-1-yl)phenylamino)-2-(pyridin-2-yl)-2,3-dihydropyrido[3,2-f][1,4]oxazepin-4(5H)-yl)acetonitrile;
N-(3-methoxy-4-(4-methyl-1H-imidazol-1-yl)phenyl)-4-methyl-2-(pyridin-2-yl)-2,3,4,5-tetrahydropyrido[3,2-f][1,4]oxazepin-8-amine;
(R)-2-(3-(3-methoxy-4-(4-methyl-1H-imidazol-1-yl)phenylamino)-6-phenyl-6,7-dihydropyrazino[2,3-f][1,4]oxazepin-8(9H)-yl)acetonitrile;
(R)—N-(3-methoxy-4-(4-methyl-1H-imidazol-1-yl)phenyl)-8-methyl-6-phenyl-6,7,8,9-tetrahydropyrazino[2,3-f][1,4]oxazepin-3-amine;
(R)-8-(3-methoxy-4-(4-methyl-1H-imidazol-1-yl)phenylamino)-N,N-dimethyl-2-phenyl-2,3-dihydropyrido[3,2-f][1,4]oxazepine-4(5H)-carboxamide;
(R)-2-(8-(3-methoxy-4-(3-methyl-1H-1,2,4-triazol-1-yl)phenylamino)-2-phenyl-2,3-dihydropyrido[3,2-f][1,4]oxazepin-4(5H)-yl)ethanol;
(R)-2-(8-(4-(4-methyl-1H-imidazol-1-yl)phenylamino)-2-phenyl-2,3-dihydropyrido[3,2-f][1,4]oxazepin-4(5H)-yl)ethanol;
(R)-2-(8-(5-methoxy-6-(4-methyl-1H-imidazol-1-yl)pyridin-3-ylamino)-2-phenyl-2,3-dihydropyrido[3,2-f][1,4]oxazepin-4(5H)-yl)ethanol;
(R)—N-(3-methoxy-4-(3-methyl-1H-1,2,4-triazol-1-yl)phenyl)-4-methyl-2-phenyl-2,3,4,5-tetrahydropyrido[3,2-f][1,4]oxazepin-8-amine;
(R)-4-cyclopropyl-N-(3-methoxy-4-(3-methyl-1H-1,2,4-triazol-1-yl)phenyl)-2-phenyl-2,3,4,5-tetrahydropyrido[3,2-f][1,4]oxazepin-8-amine;
2-(8-(3-methoxy-4-(4-methyl-1H-imidazol-1-yl)phenylamino)-2-phenyl-2,3-dihydropyrido[3,2-f][1,4]oxazepin-4(5H)-yl)ethanol;
(R)-2-(8-(3-methoxy-4-(4-methyl-1H-imidazol-1-yl)phenylamino)-2-phenyl-2,3-dihydropyrido[3,2-f][1,4]oxazepin-4(5H)-yl)acetonitrile;
(R)-4-methyl-N-(4-(3-methyl-1H-1,2,4-triazol-1-yl)phenyl)-2-phenyl-2,3,4,5-tetrahydropyrido[3,2-f][1,4]oxazepin-8-amine;
2-cyclopropyl-N-(3-methoxy-4-(4-methyl-1H-imidazol-1-yl)phenyl)-4-methyl-2,3,4,5-tetrahydropyrido[3,2-f][1,4]oxazepin-8-amine;
(R)—N-(6-methoxy-5-(1-methyl-1H-pyrazol-4-yl)pyridin-2-yl)-4-methyl-2-phenyl-2,3,4,5-tetrahydropyrido[3,2-f][1,4]oxazepin-8-amine;
2-cyclopropyl-N-(3-methoxy-4-(3-methyl-1H-1,2,4-triazol-1-yl)phenyl)-4-methyl-2,3,4,5-tetrahydropyrido[3,2-f][1,4]oxazepin-8-amine;
N-(3-methoxy-4-(3-methyl-1H-1,2,4-triazol-1-yl)phenyl)-4-methyl-2-(6-methylpyridin-2-yl)-2,3,4,5-tetrahydropyrido[3,2-f][1,4]oxazepin-8-amine;
N-(6-methoxy-5-(1-methyl-1H-pyrazol-4-yl)pyridin-2-yl)-4-methyl-2-(6-methylpyridin-2-yl)-2,3,4,5-tetrahydropyrido[3,2-f][1,4]oxazepin-8-amine;
4-methyl-N-(6-(1-methyl-1H-pyrazol-4-yl)pyridin-3-yl)-2-(6-methylpyridin-2-yl)-2,3,4,5-tetrahydropyrido[3,2-f][1,4]oxazepin-8-amine;
(R)—N-(3-methoxy-4-(4-methyl-1H-imidazol-1-yl)phenyl)-2-phenyl-2,3,4,5-tetrahydropyrido[3,2-f][1,4]oxazepin-8-amine;
(R)—N-(6-methoxy-5-(1-methyl-1H-pyrazol-4-yl)pyridin-2-yl)-2-phenyl-2,3,4,5-tetrahydropyrido[3,2-f][1,4]oxazepin-8-amine;
(R)—N-(5-(1-methyl-1H-pyrazol-4-yl)pyridin-2-yl)-2-phenyl-2,3,4,5-tetrahydropyrido[3,2-f][1,4]oxazepin-8-amine;
(R)-4-methyl-N-(6-(1-methyl-1H-pyrazol-4-yl)pyridin-3-yl)-2-phenyl-2,3,4,5-tetrahydropyrido[3,2-f][1,4]oxazepin-8-amine;
(R)-4-methyl-N-(4-(1-methyl-1H-pyrazol-4-yl)phenyl)-2-phenyl-2,3,4,5-tetrahydropyrido[3,2-f][1,4]oxazepin-8-amine; and
(R)-(1-(4-(4-methyl-2-phenyl-2,3,4,5-tetrahydropyrido[3,2-f][1,4]oxazepin-8-ylamino)phenyl)-1H-imidazol-2-yl)methanol;
   as a free base or a pharmaceutically acceptable salt thereof.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds, wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

As used herein, “tautomer” means other structural isomers that exist in equilibrium resulting from the migration of a hydrogen atom. For example, keto-enol tautomerism where the resulting compound has the properties of both a ketone and an unsaturated alcohol.

Compounds of the invention further include hydrates and solvates.

The present invention further includes isotopically-labeled compounds of the invention. An “isotopically” or “radio-labeled” compound is a compound of the invention where one or more atoms are replaced or substituted with an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

In a second aspect, the present invention provides a pharmaceutical composition comprising an effective amount of a compound according to the first aspect of the invention, or a pharmaceutically acceptable salt thereof, in association with pharmaceutically acceptable excipients, carriers or diluents.

In a third aspect, the present invention provides a compound according to the first aspect of the invention, or a pharmaceutically acceptable salt thereof, for use as a medicament.

In a further embodiment of the third aspect, the present invention provides a compound according to the first aspect, or a pharmaceutically acceptable salt thereof, for use in treating or preventing Aβ-related pathology.

In a further embodiment of the third aspect, the present invention provides a compound according to the first aspect, or a pharmaceutically acceptable salt thereof, for use in treating or preventing Aβ-related pathologies selected from the group of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In a fourth aspect, the present invention provides use of a compound according to the first aspect, or a pharmaceutically acceptable salt thereof, for manufacture of a medicament for treating or preventing an Aβ-related pathology.

In one embodiment of the fourth aspect, said Aβ-related pathologies are selected from the group of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In a fifth aspect, the present invention provides a method of treating or preventing an Aβ-related pathology in a mammal, comprising administering to said mammal a therapeutically effective amount of a compound according to the first aspect of the present invention, or a pharmaceutically acceptable salt thereof.

In a one embodiment of said fifth aspect, a method of treating or preventing an Aβ-related pathology in a mammal is provided, said method comprising administering to said mammal a therapeutically effective amount of a compound according to said first aspect, or a pharmaceutically acceptable salt thereof, and at least one cognitive enhancing agent, memory enhancing agent, acetyl choline esterase inhibitor, anti-inflammatory agents or atypical antipsychotic agents.

In another embodiment of said fifth aspect, said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula (I) or a pharmaceutically acceptable salt thereof for the therapeutic treatment (including prophylactic treatment) of mammals including humans, it is normally formulated in accordance with standard pharmaceutical practice as a pharmaceutical composition.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

The quantity of the compound to be administered will vary for the patient being treated and will vary from about 100 ng/kg of body weight to 100 mg/kg of body weight per day and preferably will be from 10 pg/kg to 50 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

The treatment of Aβ-related pathology defined herein may be applied as a sole therapy or may involve, in addition to the compound of the invention, conjoint treatment with conventional chemotherapy of value in treating one or more disease conditions referred to herein. Such conventional chemotherapy may include one or more of the following categories of agents: acetyl cholinesterase inhibitors, anti-inflammatory agents, cognitive and/or memory enhancing agents or atypical antipsychotic agents. Cognitive enhancing agents, memory enhancing agents and acetyl choline esterase inhibitors includes, but not limited to, onepezil (Aricept), galantamine (Reminyl or Razadyne), rivastigmine (Exelon), tacrine (Cognex) and memantine (Namenda, Axura or Ebixa). Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

Such conjoint treatment may be achieved by way of the simultaneous, sequential or separate dosing of the individual components of the treatment. Such combination products employ the compounds of the invention.

Additional conventional chemotherapy may include one or more of the following categories of agents:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.
(ii) atypical antipsychotics including for example quetiapine and pharmaceutically active isomer(s) and metabolite(s) thereof.
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof.
(v) anticonvulsants including for example carbamazepine, valproate, lamotrogine, gabapentin and equivalents and pharmaceutically active isomer(s) and metabolite(s) thereof.
(vi) Alzheimer's therapies including for example donepezil, memantine, tacrine and equivalents and pharmaceutically active isomer(s) and metabolite(s) thereof.
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.
(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.
(ix) stroke therapies including for example abciximab, activase, NXY-059, citicoline, crobenetine, desmoteplase, repinotan, traxoprodil and equivalents and pharmaceutically active isomer(s) and metabolite(s) thereof.
(x) urinary incontinence therapies including for example darafenacin, falvoxate, oxybutynin, propiverine, robalzotan, solifenacin, tolterodine and equivalents and pharmaceutically active isomer(s) and metabolite(s) thereof
(xi) neuropathic pain therapies including for example gabapentin, lidoderm, pregablin and equivalents and pharmaceutically active isomer(s) and metabolite(s) thereof.
(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.
(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.
(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference.

### METHODS OF PREPARATION FOR COMPOUNDS OF THE PRESENT INVENTION

Preparation of the compounds of the present invention will be illustrated below.

In each of the following preparation methods, when a defined group changes under reaction conditions or is not suitable for carrying out the method, the preparation can be easily carried out by subjecting the group to a procedure conventionally employed in organic synthetic chemistry, such as protection and/or deprotection of a functional group (for example see, Protection Groups in Organic Synthesis, T. W. Green, Wiley & Sons Inc. (1999)).

Where necessary, the order of reaction process steps such as introduction of substituents can be altered. Solvent, temperature, pressure, and other reaction conditions may readily be selected by the skilled person. Starting materials are commercially available or readily prepared by one skilled in the art. Compounds of formula (I) can be prepared, for example, using the Methods of Preparation 1 to 4. In the method of preparation below, PG represents a protective group or a substituent. PG can be replaced or exchanged prior to, during or immediately following the process mentioned below.

### Method of Preparation 1

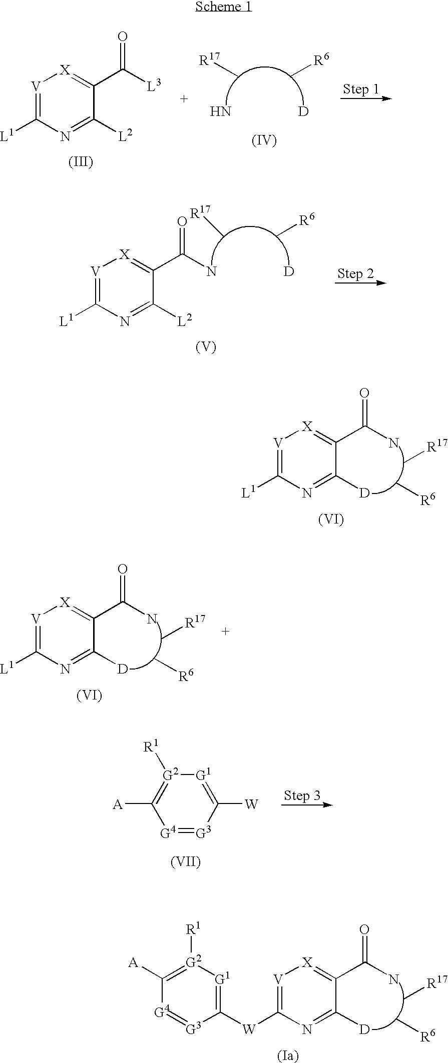

wherein
L¹ and L² are a halogen; L³ is Cl, F, or OH;
R¹, R⁶, R¹⁷, G¹, G², G³, G⁴, A, V, X, W are as defined in claim 1;
D is as defined for the compound of Formula (III) in claim 2;
Compound of formula (IV) is optionally substituted;

### Step 1

In this step, an acid of formula (III) and an amine of formula (IV) are subjected to a dehydrative condensation to give a compound represented by the formula (V).

The dehydrative condensation is performed by a method known per se, for example, a method using a condensation agent or a method using a reactive derivative and the like. Examples of the condensation reagent used include dicyclohexylcarbodiimide, diisopropylcarbodiimide or O-benzotriazol-1-yl-N,N,N′,N′-tetra-methyluronium hexafluorophosphate. They may be used alone or in combination of additives (e.g., N-hydroxysuccinimide, 1-hydroxybenzotriazol). The reaction above is generally performed in a solvent that does not adversely influence the reaction (e.g., dichloromethane, DMF, THF, pyridine) and an appropriate base can also be present (e.g., triethylamine, diisoprylmethylamine, sodium hydroxide).

Reactive derivatives such as acid halides and active esters also react with amine of formula (IV) to form compound of formula (V). The reactive derivatives are prepared under standard condition know by the skill in the art. Acid of formula (III) is converted to an acid halide using reagents such as thionyl chloride, oxalyl chloride, phosphorus trichloride and the like neat or in presence of a suitable solvent (dichloromethane, THF, dioxane and the like). The reactive derivative of formula (V) and amine of formula (IV) are mixtured generally in presence of a base (triethylamine, diisopropylamine, sodium hydroxide) in a suitable solvent (THF, dichloromethane, dioxane and the like) at an appropriate temperature (−50° C. to 100° C.) to afford compound of formula (V).

### Step 2

Compound of formula (V) is converted to a compound of formula (VI) via an intramolecular ring closure reaction. The reaction is generally performed in presence of a base and in a suitable solvent (ether, THF, dioxane, DMF and the like). Examples of base include metal hydride (potassium hydride, sodium hydride), inorganic base (lithium hydroxide, sodium hydroxide, potassium hydroxide, sodium hydrogen carbonate, hydrogen carbonate) and organic base (triethylamine, diisopropylamine, pyridine, sodium ethoxide,). The reaction temperature is, for example, about −50° C. to about 200° C.

### Step 3

Compound of formula (VI) reacts with nucleophile of formula (VII) under thermal heating or under cross-coupling conditions to form compound of formula (Ia)

Heating compound of formula (VI) in presence of a suitable nucleophile of formula (VI) afford compounds of formula (Ia). The reaction is generally performed in presence of a base and in a suitable solvent (ether, THF, dioxane, DMF and the like). Examples of base include metal hydride (potassium hydride, sodium hydride), inorganic base (lithium hydroxide, sodium hydroxide, potassium hydroxide, sodium hydrogen carbonate, hydrogen carbonate) and organic base (triethylamine, diisopropylamine, pyridine, sodium ethoxide). The conditions for displacing the leaving group in compound of formula (IV) will depend on the nature and the reactivity of the nucleophile of formula (VII). The reaction temperature is, for example, about −50° C. to about 200° C.

Cross-coupling reaction is an alternative method for converting a compound of formula (VI) into a compound of formula (Ia). Compounds of formula (VI) and of formula (VII) are heated in presence of a catalyst (such as Pd(OAc)₂ and Pd(dba)₂, a ligand (such as BINAP, dppf and Xantphos), a suitable base (such as potassium tert-butoxide and CsCO₃) in a suitable solvent (see for examples Accounts of Chemical Research, 2002, 35, 717; and J. Am. Chem. Soc. 2003, 125, 6653).

### Method of Preparation 2

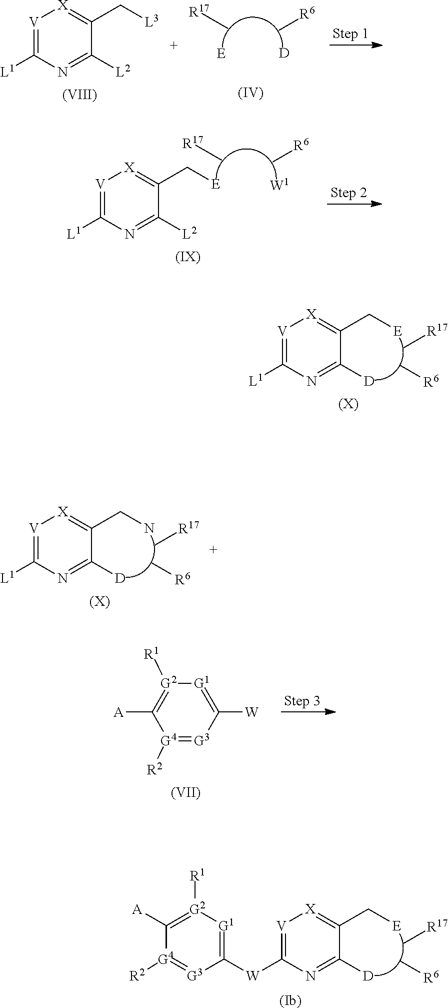

L¹ and L² are a halogen; L³ is Cl, Br, I or OS(O)₂CH₃; W¹ is O or N;
R¹, R², R¹⁷, R⁶, G¹, G², G³, G⁴, A, V, X, W are as defined in claim 1;
D and E are as defined for the compound of Formula (III) in claim 2;
Compound of formula (IV) is optionally substituted;

### Step 1

Compound of formula (IX) is obtained by reacting a compound of formula (VIII) with a amine of formula (IV) as depicted below. The reaction is carried out in a suitable solvent (ethanol, methanol, DMF, dioxane) optionally in presence of a base such as a tertiary amine (triethylamine, diisopropylamine) or an inorganic base (potassium carbonate, sodium carbonate) at a temperature compromise between room temperature and 200° C. Addition of a catalytic amount of potassium iodine can be advantageous.

### Step 2

Compound of formula (IX) is converted to a compound of formula (X) via an is intramolecular ring closure reaction. The reaction is generally performed in presence of a base and in a suitable solvent (ether, THF, dioxane, DMF and the like). Examples of base include metal hydride (potassium hydride, sodium hydride), inorganic base (lithium hydroxide, sodium hydroxide, potassium hydroxide, sodium hydrogen carbonate, hydrogen carbonate) and organic base (triethylamine, diisopropylamine, pyridine, sodium ethoxide). The reaction temperature is, for example, about −78° C. to about 200° C.

### Step 3

Compound of formula (X) reacts with nucleophile of formula (VII) under thermal heating or under cross-coupling conditions to form compound of formula (Ib)

Heating compound of formula (X) in presence of a suitable nucleophile of formula (VI) afford compounds of formula (Ib). The reaction is generally performed in presence of a base and in a suitable solvent (ether, THF, dioxane, DMF and the like). Examples of base include metal hydride (potassium hydride, sodium hydride), inorganic base (lithium hydroxide, sodium hydroxide, potassium hydroxide, sodium hydrogen carbonate, hydrogen carbonate) and organic base (triethylamine, diisopropylamine, pyridine, sodium ethoxide). The conditions for displacing the leaving group in compound of formula (IV) will depend on the nature and the reactivity of the nucleophile of formula (VII). The reaction temperature is, for example, about −50° C. to about 200° C.

Cross-coupling reaction is an alternative method for converting a compound of formula (VI) into a compound of formula (Ia). Compounds of formula (VI) and of formula (VII) are heated in presence of a catalyst (such as Pd(OAc)₂ and Pd(dba)₂, a ligand (such as BINAP, dppf and Xantphos), a suitable base (such as potassium tert-butoxide and CsCO₃) in a suitable solvent (see for examples Accounts of Chemical Research, 2002, 35, 717; and J. Am. Chem. Soc. 2003, 125, 6653).

### Method of Preparation 3

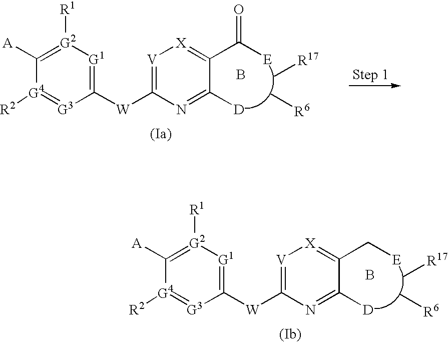

R¹, R², R⁶, R¹⁷, G¹, G², G³, G⁴, A, B, V, X, W are as defined for the compound of Formula (I);
D and E are as defined for the compound of Formula (III) in claim 2;

Compound of formula (Ib) can be prepared by reducing the amide functionality in compound of formula (Ia). Compound of formula (Ia) is allowed to reaction with a reducing agent (such as lithium aluminium hydride, borane, carbonylhydrotris(triphenylphosphine)rhodium (I) in combined with diphenylsilane) at a temperature between −50° C. and 200° C. and in a suitable solvent (such as THF, diethylether).

### Method of Preparation 4

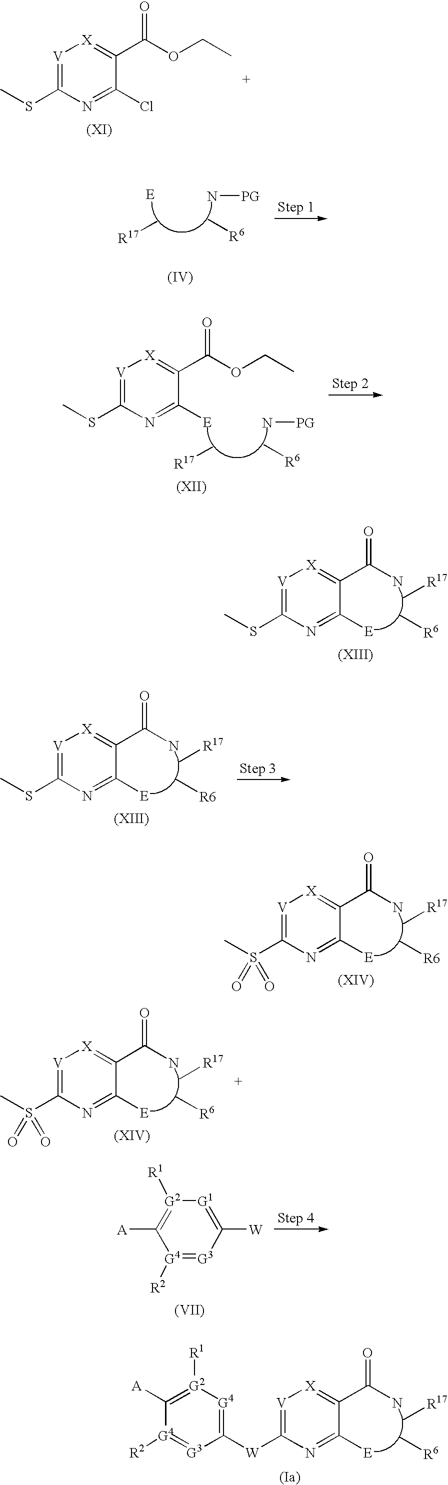

R¹, R², R⁶, R¹⁷, G¹, G², G³, G⁴, A, V, X, W are as defined for the compound of Formula (I);
E is as defined for the compound of Formula (III) in claim 2;
Compound of formula (IV) is optionally substituted;

### Step 1

Compound of formula (XII) is prepared by reaction compound of formula (XI) with a suitable nucleophile of formula (IV). The reaction is carried out at a temperature between −50° C. and 150° C. in a suitable solvent (such as DMF, THF, tent-butanol) preferably in presence of a base (such as sodium acetate, triethylamine, pyridine, potassium carbonate, potassium hydrogen carbonate).

### Step 2

Ester of formula (XII) is hydrolyzed under basic using a suitable base (such as sodium hydroxide and lithium hydroxide) in a suitable solvent (such as water, water/THF mixture and water/methanol mixture) at temperature between 0° C. and 100° C. The carboxylic acid may cyclized spontaneously into the lactam of formula (XIII). Alternatively, the carboxylic acid can be converted into the lactam of formula (XIII) using standard peptide coupling conditions described in the method of preparation 1 (1^{st} step).

### Step 3

Conversion of compound of formula (XIII) to the corresponding sulphone of formula (XIV) can be obtained by reacting a compound of formula (XIII) with an suitable oxidation agent (such as m-chloroperbenzoic acid) in a suitable solvent (such as dichloromethane).

### Step 4

Displacement of a compound of formula (XIV) with a suitable nucleophile of formula (VII) such as amine and alcohol provides compound of formula (Ia).

### Method of Preparation 5

### W¹ is O and N;

R¹, R², R⁶, R¹⁷, A, B, G¹, G², G³, G⁴, W and Y are as defined for the compound of Formula (II) in claim 1;
Compound of formula (XV) is optionally substituted;

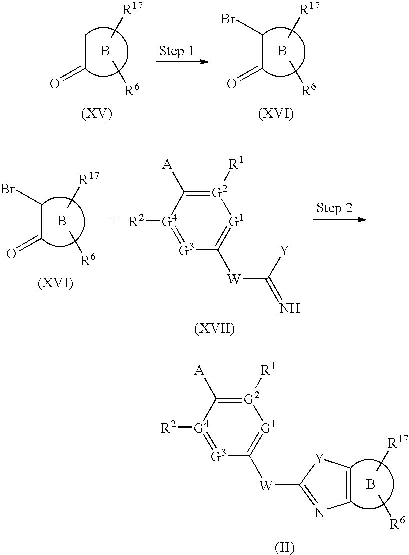

### Step 1

alpha-Halo-ketones of formula (XVI) can be prepared from ketones of formula (XV) through halogenation with either elemental halogen or a halide-transferring reagent (such as N-bromosuccinimide). Alternatively, the ketones of formula (XV) are treated with a base (such as LDA or LiTMP) at a temperature below room temperature. This treatment is followed by the addition of dibromotetrachlororethane or hexachloroethane whereby ketones of formula (XVI) is obtained.

### Step 2

The reactants of formula (XVII) are condensed with the alpha-halo-ketones of formula (XVI) in an inert solvent (such as ethanol) at room temperature or at elevated temperature in presence or absence of a base (such as potassium carbonate or triethylamine), whereby the compounds of Formula (II) are obtained.

### General Methods

All solvents used were of analytical grade and commercially available anhydrous solvents were routinely used for reactions. Starting materials used were available from commercial sources, or prepared according to literature procedures.

Microwave heating was performed in a Creator, Initiator or Smith Synthesizer Single-mode microwave cavity producing continuous irradiation at 2450 MHz It is understood that microwaves can be used for the heating of reaction mixtures.

NMR spectroscopy was performed on a Bruker DPX400 NMR spectrometer operating at 400 MHz for ¹H, 376 MHz for ¹⁹F, and 100 MHz for ¹³C, equipped with a 4-nucleus probe-head with Z-gradients. Alternatively, NMR spectroscopy was performed on a Bruker 500 MHz Avance III NMR spectrometer, operating at 500 MHz for ¹H, 125 MHz for ¹³C, and 50 MHz for ¹⁵N equipped with a 5 mm TCI cryogenically cooled probe-head with Z-gradients. Alternatively, NMR spectroscopy was performed on a Bruker DRX600 NMR spectrometer, operating at 600 MHz for ¹H, 150 MHz for ¹³C and 60 MHz for ¹⁵N, equipped with a 5 mm TXI probe-head with Z-gradients. Alternatively, NMR spectroscopy was performed on a Varian Mercury Plus 400 NMR Spectrometer equipped with a Varian 400 ATB PFG probe, operating at 400 MHz for ¹H and 100 MHz for ¹³C.

The following reference signals were used: the middle line of (CD₃)₂SO δ 2.50 (¹H), δ 39.51 (¹³C); the middle line of CD₃OD δ 3.31 (¹H) or δ 49.15 (¹³C); CDCl₃ δ 7.26 (¹H) and the middle line of CDCl₃ δ 77.16 (¹³C); if the solvent contained 0.03% to 0.05% v/v tetramethylsilane, δ 0.00 (¹H and ¹³C); unless otherwise indicated.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm. Or LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode. Separation was performed using a Gemini column C18, 3.0 mm×50 mm, 3 μm, (Phenomenex) and run at a flow rate of 1 ml/min. A linear gradient was used starting at 100% A (A: 10 mM ammonium acetate in 5% methanol) and ending at 100% B (methanol). The column oven temperature was set to 40° C.

LC-MS analyses were performed on a LC-MS system consisting of a Waters Alliance 2795 HPLC, a Waters PDA 2996 diode array detector, a Sedex 75 ELS detector and a ZQ single quadrupole mass spectrometer. The mass spectrometer was equipped with an ES ion source operated in positive or negative ion mode. The capillary voltage was set to 3.2 kV and the cone voltage to 30 V, respectively. The mass spectrometer was scanned between m/z 100-700 with a scan time of 0.3 seconds. The diode array detector scanned from 200-400 nm. The temperature of the ELS detector was adjusted to 40° C. and the pressure was set to 1.9 bar. Separation was performed on an X-Terra MS C8, 3.0 mm×50 mm; 3.5 μm (Waters) run at a flow rate of 1 mL/min. A linear gradient was applied starting at 100% A (A: 10 mM NH₄OAc in 5% MeCN, or 8 mM HCOOH in 5% MeCN) ending at 100% B (B: MeCN). The column oven temperature was set to 40° C.

Thin layer chromatography (TLC) was performed on Merck TLC-plates (Silica gel 60 F254) and UV visualized the spots. Flash chromatography was performed on a Combi Flash® Companion™ using RediSep™ normal-phase flash columns or using Merck Silica gel 60 (0.040-0.063 mm). Typical solvents used for flash chromatography were mixtures of chloroform/methanol, DCM/methanol, heptane/ethyl acetate, chloroform/methanol/ammonia (aq.) and DCM/methanol/NH₃ (aq.).

Preparative chromatography was run on a Waters FractionLynx system with a Autosampler combined Automated Fraction Collector (Waters 2767), Gradient Pump (Waters 2525), Column Switch (Waters CFO) and PDA (Waters 2996). The column was a) XTerra® Prep MS C8 10 μm OBD™ 19×300 mm or b) XTerra® Prep MS C8 10 μm OBD™ 30×150 mm, with guard column; XTerra® Prep MS C8 10 μm 19×10 mm Cartridge. A gradient from 100% A (95% 0.1 M NH₄OAc in MilliQ water and 5% MeCN) to 100% B (100% MeCN) was applied for LC-separation at flow rate a) 20 mL/min or b) 40 mL/min. The PDA was scanned from 210-350 nm. UV triggering determined the fraction collection.

Preparative chromatography for chiral separation was run on a Berger Multigram II system. The column was Chiralcpak OD, 4.6×250 mm using the mobile phase 60% EtOH+0.1% DEA, 40% heptane; or Reprosil-NR, 4.6×250 mm; 5 μm using the mobile phase 35% MeOH+0.1% DEA/65% CO2 applied at a flow rate of 50 mL/min; or Chiracel 4.6×250 mm; 5 μm using the mobile phase 45% MeOH+0.1% DEA/55% CO2 applied at a flow rate of 50 mL/min.

Compounds have been named using CambridgeSoft MedChem ELN v2.1.

### ABBREVIATIONS

app apparent
aq. Aqueous
Ar Argon (or Argon atmosphere)
br broadened
BINAP 2,2′-bis(diphenylphosphino)-1,1′-binaphthyl
CI chemical ionization
δ chemical shift in parts per million (ppm) downfield from the standard
d doublet

### DCM DCM

### DIPEA N,N-diisopropylethylamine

### DMA N,N-dimethylacetamide

### DMF N,N-dimethylformamide

DME 1,2-dimethoxyethane
DMSO dimethyl sulfoxide
dppf 1,1′-bis(diphenylphosphino)ferrocene
EI electron impact
eq equivalents
ES electro-spray
ELS electro-spray
Et₂O diethyl ether
EtOH ethanol
HCl hydrochlorid acid
HBTU O-Benzotriazol-1-yl-N,N,N′,N′-tetra-methyluronium hexafluorophosphate
HPLC high performance liquid chromatography
LC liquid chromatography
m multiplet
MeCN acetonitrile
MeOH methanol
MS mass spectroscopy
NMR nuclear magnetic resonance
o.n. Over-night
Pd(dba)₂ bis(dibenzylideneacetone)palladium
Pd(OAc)₂ palladium (II) acetate
Pd(PPh₃)₄ tetrakis(triphenylphosphine)palladium(0)
PDA photodiode array detector
prep. Preparative
q quartet
r.t. Room temperature (ca 21-25° C.)
s singlet
t triplet
THF tetrahydrofuran
TLC thin layer chromatography
UV ultra violet
Xantphos 4,5-bis(diphenylphosphino)-9,9-dimethylxanthene

### EXAMPLES

Below follows a number of non-limiting examples of compounds of the invention.

### Example 1

### 4-methyl-8-(4-(3-methyl-1,2,4-oxadiazol-5-yl)phenylamino)-2-phenyl-3,4-dihydropyrido[3,2-f][1,4]oxazepin-5(2H)-one

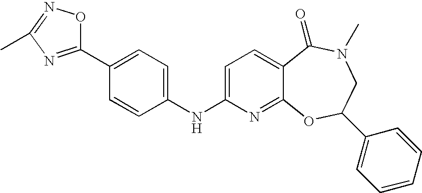

A mixture of 8-chloro-4-methyl-2-phenyl-3,4-dihydropyrido[3,2-f][1,4]oxazepin-5(2H)-one (85 mg, 0.29 mmol), 4-(3-methyl-1,2,4-oxadiazol-5-yl)aniline (72.2 mg, 0.41 mmol), Pd(OAc)₂ (6.61 mg, 0.03 mmol), biphenyl-2-yldicyclohexylphosphine (10.32 mg, 0.03 mmol) and Cs₂CO₃ (288 mg, 0.88 mmol) in DME (2 mL) was heated to 100° C. in a microwave reactor for 1 h. The reaction mixture was filtered, the solvent was evaporated and the residue was purified by HPLC to give 23.7 mg of product as a solid (19% Yield).

¹H NMR (400 MHz, DMSO-d₆) δ ppm 8.04 (d, 1H) 7.98 (m, 4H) 7.50-7.57 (m, 2H) 7.40-7.47 (m, 2H) 7.33-7.40 (m, 1H) 6.77 (d, 1H) 5.86 (t, 1H) 3.86 (d, 2H) 2.92 (s, 3H) 2.38 (s, 3H) 10.02 (s, 1H). MS m/z 428 [M+H] 426 [M−H].

### Example 1a

### 8-chloro-4-methyl-2-phenyl-3,4-dihydropyrido[3,2-f][1,4]oxazepin-5(2H)-one

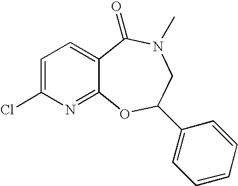

A solution of 2,6-dichloro-N-(2-hydroxy-2-phenylethyl)-N-methylnicotinamide (5.50 g, 16.91 mmol) in THF (50 mL) was slowly added to a ice-water cooled suspension of NaH (0.449 g, 17.76 mmol) in THF (50 mL). The resulting mixture was stirred over night while it was allowed to slowly come to room temperature. The reaction mixture was diluted with ethyl acetate, washed with saturated aqueous NaHCO₃, water and brine, then dried over MgSO₄ and evaporated. The residue was purified by column chromatography on Silica, using gradient elution with increasing concentration of methanol from 0 to 5% in dichloromethane, to give 2.66 g of the title product as a solid (54% Yield). ¹H NMR (400 MHz, DMSO-d₆) δ ppm 8.21 (d, 1H) 7.35-7.49 (m, 6H) 5.91 (m, 1H) 3.89-3.97 (m, 1H) 3.82-3.89 (m, 1H) 2.93 (s, 3H). MS m/z 289, 291 [M+H].

### Example 1b

### 2,6-dichloro-N-(2-hydroxy-2-phenylethyl)-N-methylnicotinamide

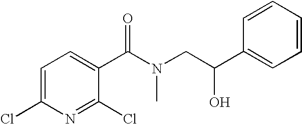

2,6-Dichloronicotinoyl chloride (4.02 g, 19.10 mmol) was dissolved in THF (25 mL) and added dropwise to a solution of 2-(methylamino)-1-phenylethanol (3.47 g, 22.92 mmol) and Et₃N (5.32 mL, 38.20 mmol) in THF (25 mL). The resulting slurry was stirred at room temperature for 16 h. The reaction mixture was diluted with ethyl acetate, washed with 1 M HCl, water and saturated aqueous NaHCO₃, dried over MgSO₄ and evaporated to give 6.259 g of the title product as a solid (quantitative). The product was used without further purification in the subsequent reaction. MS m/z 307, 309 [M−OH] 325, 327 [M+H] 383, 385 [M+OAc]

### Example 2

### 4-methyl-8-(4-(2-methyl-1H-imidazol-1-yl)phenylamino)-2-phenyl-3,4-dihydropyrido[3,2-f][1,4]oxazepin-5(2H)-one

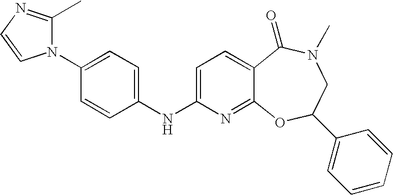

The title compound was prepared in an analogous procedure as described in Example 1 using 4-(2-methyl-1H-imidazol-1-yl)aniline as starting material (42% Yield). ¹H NMR (400 MHz, DMSO-d₆) δ ppm 9.74 (s, 1H) 8.00 (d, J=8.34 Hz, 1H) 7.82-7.89 (m, 2H) 7.50-7.55 (m, 2H) 7.39-7.46 (m, 2H) 7.32-7.39 (m, 3H) 7.22 (d, J=1.39 Hz, 1H) 6.88 (d, J=1.39 Hz, 1H) 6.69 (d, J=8.59 Hz, 1H) 5.79-5.84 (m, 1H) 3.82-3.88 (m, 2H) 2.91 (s, 3H) 2.26 (s, 3H). MS m/z 426 [M+H] 424 [M−H].

### Example 3

### 4-methyl-8-(4-(1,3-oxazol-5-yl)phenylamino)-2-phenyl-3,4-dihydropyrido[3,2-f][1,4]oxazepin-5(2H)-one

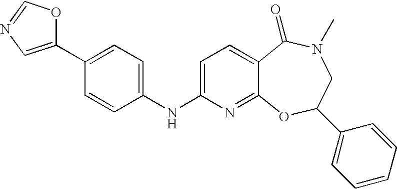

The title compound was prepared in an analogous procedure as described in Example 1 using 4-(oxazol-5-yl)aniline as starting material (7% Yield). ¹H NMR (400 MHz, DMSO-d₆) δ ppm 9.72 (s, 1H) 8.37 (s, 1H) 7.99 (d, 1H) 7.84 (m, 2H) 7.65 (m, 2H) 7.51-7.56 (m, 3H) 7.40-7.46 (m, 2H) 7.34-7.39 (m, 1H) 6.70 (d, 1H) 5.82 (dd, 1H) 3.79-3.89 (m, 2H) 2.93 (s, 3H). MS m/z 413 [M+H] 411 [M−H].

### Example 4

### 8-(3-methoxy-4-(4-methyl-1H-imidazol-1-yl)phenylamino)-4-methyl-2-phenyl-3,4-dihydropyrido[3,2-f][1,4]oxazepin-5(2H)-one

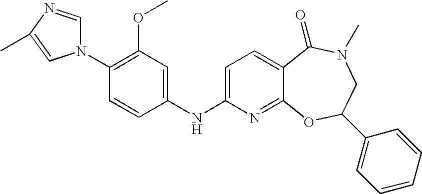

The title compound was prepared in an analogous procedure as described in Example 1 using 3-methoxy-4-(4-methyl-1H-imidazol-1-yl)aniline hydrochloride as starting material (40% Yield). ¹H NMR (400 MHz, DMSO-d₆) δ ppm 9.91 (s, 1H) 8.75 (br. s., 1H) 8.02 (d, J=8.51 Hz, 1H) 7.90 (d, J=1.73 Hz, 1H) 7.51-7.57 (m, 2H) 7.46 (br. s., 1H) 7.32-7.44 (m, 5H) 6.74 (d, J=8.35 Hz, 1H) 5.83 (dd, J=7.80, 2.13 Hz, 1H) 3.84-3.91 (m, 1H) 3.75 (s, 3H) 3.70-3.81 (m, 3H) 2.98 (s, 3H) 2.27 (s, 3H). MS m/z 456 [M+H] 454 [M−H].

### Example 5

### 8-(4-(1H-imidazol-1-yl)phenylamino)-4-methyl-2-phenyl-3,4-dihydropyrido[3,2-f][1,4]oxazepin-5(2H)-one

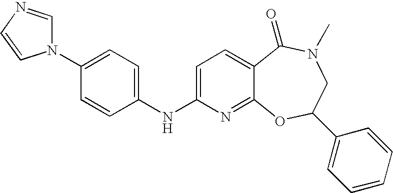

The title compound was prepared in an analogous procedure as described in Example 1 using 4-(1H-imidazol-1-yl)aniline as starting material (38% Yield). ¹H NMR (400 MHz, DMSO-d₆) δ ppm 9.68 (s, 1H) 8.15-8.19 (m, 1H) 7.99 (d, J=8.34 Hz, 1H) 7.82-7.88 (m, 2H) 7.67 (t, J=1.33 Hz, 1H) 7.51-7.60 (m, 4H) 7.40-7.46 (m, 2H) 7.33-7.39 (m, 1H) 7.08 (t, J=1.07 Hz, 1H) 6.67 (d, J=8.59 Hz, 1H) 5.79-5.84 (m, 1H) 3.78-3.91 (m, 2H) 2.93 (s, 3H). MS m/z 412 [M+H] 410 [M−H].

### Example 6

### 4-methyl-8-(6-(2-methyl-1H-imidazol-1-yl)pyridin-3-ylamino)-2-phenyl-3,4-dihydropyrido[3,2-f][1,4]oxazepin-5(2H)-one

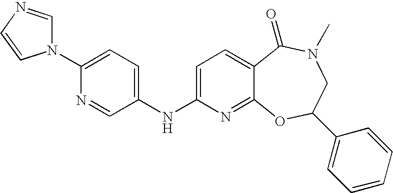

The title compound was prepared in an analogous procedure as described in Example 1 using 6-(2-methyl-1H-imidazol-1-yl)pyridin-3-amine as starting material (55% Yield). ¹H NMR (600 MHz, DMSO-d₆) δ ppm 9.89 (s, 1H) 8.84 (d, 1H) 8.40 (dd, 1H) 8.05 (d, 1H) 7.53 (m, 3H) 7.43 (m, 3H) 7.36 (m, 1H) 6.88 (s, 1H) 6.72 (d, 1H) 5.82 (m, 1H) 3.85 (m, 2H) 2.92 (s, 3H) 2.44 (s, 3H). MS m/z 427 [M+H] 425 [M−H].

### Example 7

### N-(3-methoxy-4-(4-methyl-1H-imidazol-1-yl)phenyl)-4-methyl-2-phenyl-2,3,4,5-tetrahydropyrido[3,2-f][1,4]oxazepin-8-amine

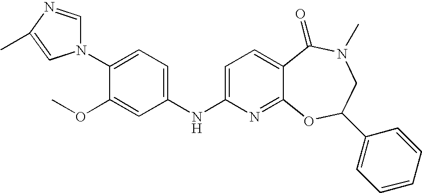

8-Chloro-4-methyl-2-phenyl-2,3,4,5-tetrahydropyrido[3,2-f][1,4]oxazepine (60.0 mg, 0.22 mmol), 3-methoxy-4-(4-methyl-1H-imidazol-1-yl)aniline (57.7 mg, 0.28 mmol), palladium (II) acetate (4.90 mg, 0.02 mmol), 2-(dicyclohexylphosphino)biphenyl (7.65 mg, 0.02 mmol) and CS₂CO₃ (213 mg, 0.66 mmol) were weighed into a microwave vial. The vial was capped and flushed with argon. DME (4 mL) was added and the mixture was heated to 100° C. in a microwave apparatus for 1 h. The cooled reaction mixture was diluted with dichloromethane and methanol, filtered through a plug of Celite and the solvents were evaporated. The residue was purified by HPLC to give 36 mg of the title product (37% Yield).

¹H NMR (600 MHz, DMSO-d₆) δ ppm 9.25 (s, 1H) 7.67 (m, 1H) 7.60 (d, 1H) 7.54 (d, 1H) 7.49 (m, 2H) 7.40 (m, 2H) 7.33 (m, 1H) 7.17 (m, 2H) 6.99 (m, 1H) 6.60 (d, 1H) 5.10 (m, 1H) 3.69 (m, 5H) 3.05 (m, 1H) 2.98 (m, 1H) 2.36 (s, 3H) 2.12 (s, 3H).

MS m/z 442 [M+H]⁺440 [M−H]⁻.

### Example 7a

### 8-Chloro-4-methyl-2-phenyl-2,3,4,5-tetrahydropyrido[3,2-f][1,4]oxazepine

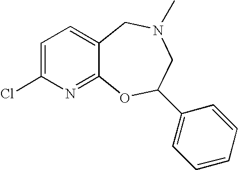

A 60% dispersion of NaH (58.4 mg, 1.46 mmol) in mineral oil was washed with hexane under argon atmosphere. Residual hexane was evaporated at reduced pressure and the NaH was suspended in THF (2 mL). To this was added a solution of 2-(((2,6-dichloropyridin-3-yl)methyl)(methyl)amino)-1-phenylethanol (413 mg, 1.33 mmol) in THF (3 mL). The resulting mixture was stirred at room temperature for 16 h. The reaction mixture was diluted with ethyl acetate and washed with water and brine, dried over Na₂SO₄ and evaporated to give 352 mg of the title product (97% Yield).

¹H NMR (600 MHz, DMSO-d₆) δ ppm 7.81 (d, 1H) 7.45-7.48 (m, 2H) 7.39-7.43 (m, 2H) 7.33-7.37 (m, 1H) 7.25 (d, 1H) 5.17-5.21 (m, 1H) 3.86 (d, 1H) 3.78 (d, 1H) 3.04-3.10 (m, 1H) 2.98-3.02 (m, 1H) 2.37 (s, 3H).

MS m/z 275, 277 [M+H]⁺.

### Example 7b

### 2-(((2,6-Dichloropyridin-3-yl)methyl)(methyl)amino)-1-phenylethanol

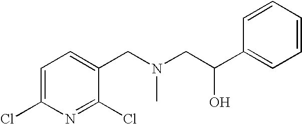

2-(methylamino)-1-phenylethanol (227 mg, 1.50 mmol) was added to a solution of 2,6-dichloro-3-(chloromethyl)pyridine (281 mg, 1.43 mmol) and TEA (0.299 mL, 2.15 mmol) in DMF (2 mL). The mixture was stirred at room temperature for 20 h. The mixture was diluted with water and extracted with ethyl acetate. The combined extracts were washed with water and brine, dried over MgSO₄ and evaporated to give 415 mg of the title product (93% Yield).

¹H NMR (600 MHz, DMSO-d₆) δ ppm 7.84 (d, 1H) 7.47 (d, 1H) 7.27-7.33 (m, 4H) 7.21-7.25 (m, 1H) 5.13 (d, 1H) 4.71-4.76 (m, 1H) 3.64 (s, 2H) 2.62 (dd, 1H) 2.51-2.55 (m, 1H) 2.28 (s, 3H).

MS m/z 311, 313, 315 [M+H]⁺.

### Example 8

### 4-Methyl-N-(4-(2-methyl-1H-imidazol-1-yl)phenyl)-2-phenyl-2,3,4,5-tetrahydropyrido[3,2-f][1,4]oxazepin-8-amine

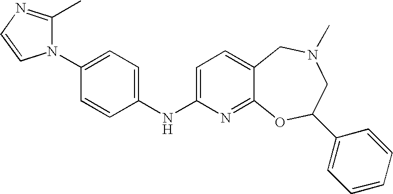

To a mixture of 4-methyl-8-(4-(2-methyl-1H-imidazol-1-yl)phenylamino)-2-phenyl-3,4-dihydropyrido[3,2-f][1,4]oxazepin-5(2H)-one (223 mg, 0.52 mmol), lithium chloride (22.22 mg, 0.52 mmol) and THF (4 mL) was added 1 M BH₃. THF (1.572 mL, 1.57 mmol). The mixture was stirred for 1 h at room temperature. Essentially no reaction had occurred as revealed by LCMS. The mixture was heated to reflux for 5 h. One more equivalent of 1 M borane tetrahydrofuran complex (0.524 mL, 0.52 mmol) was added and the mixture was refluxed for another 4 h. Still an additional equivalent of 1 M borane tetrahydrofuran complex (0.524 mL, 0.52 mmol) was added and the mixture was refluxed for 20 h. To the cooled reaction mixture was added 1.25 M HCl in methanol (10 mL, 12.5 mmol) and the mixture was stirred for 1 h and then concentrated at reduced pressure. The residue was shaken with 1 M aqueous Na₂CO₃ and dichloromethane, the organic layer was washed with saturated aqueous NaHCO₃, dried over Na₂SO₄ and concentrated. The residue was purified by three runs on HPLC to give 12.9 mg of the title product (6% Yield).

¹H NMR (600 MHz, DMSO-d₆) δ ppm 9.24 (s, 1H) 7.68 (m, 2H) 7.54 (d, 1H) 7.48 (m, 2H) 7.41 (m, 2H) 7.34 (m, 1H) 7.26 (m, 2H) 7.16 (m, 1H) 6.84 (d, 1H) 6.61 (d, 1H) 5.08 (m, 1H) 3.69 (m, 2H) 2.97 (m, 2H) 2.36 (s, 3H) 2.22 (s, 3H).

MS m/z 412 [M+H]⁺410 [M−H]⁻.

### Example 9

### (R)—N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine

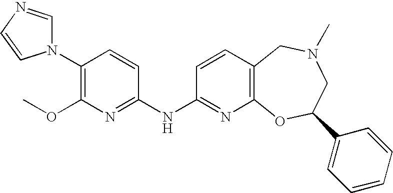

A solution of (R)-8-chloro-4-methyl-2-phenyl-2,3,4,5-tetrahydropyrido[3,2-f][1,4]oxazepine (83 mg, 0.30 mmol) and 6-methoxy-5-(4-methyl-1H-imidazol-1-yl)pyridin-2-amine (62.0 mg, 0.30 mmol) in DME (3 mL) was added to a microwave vial containing palladium (II) acetate (6.82 mg, 0.03 mmol), 2-(dicyclohexylphosphino)biphenyl (10.64 mg, 0.03 mmol) and CS₂CO₃ (297 mg, 0.91 mmol) under an atmosphere of argon. The resulting mixture was heated to 100° C. in a microwave apparatus for 2 h. The reaction mixture was diluted with dichloromethane and methanol and filtered through a plug of Celite. The solvents were evaporated and the to residue was purified by preparative HPLC to give 21 mg of the title product (16% Yield).

¹H NMR (500 MHz, DMSO-d₆) δ ppm 9.87 (s, 1H) 7.66 (m, 4H) 7.48 (m, 2H) 7.40 (m, 2H) 7.33 (m, 1H) 7.05 (m, 1H) 7.01 (d, 1H) 5.10 (dd, 1H) 3.94 (s, 3H) 3.73 (q, 2H) 3.00 (m, 2H) 2.37 (s, 3H) 2.13 (d, 3H).

MS m/z 443 [M+H]⁺441 [M−H]⁻.

### Example 9a

### (R)-8-chloro-4-methyl-2-phenyl-2,3,4,5-tetrahydropyrido[3,2-f][1,4]oxazepine

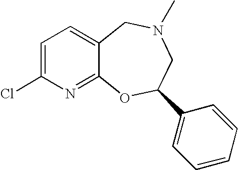

A 60% dispersion of sodium hydride (84 mg, 2.10 mmol) in mineral oil was washed with hexane under argon atmosphere and residual hexane was evaporated at reduced pressure.

The dry sodium hydride was suspended in THF (2 mL) under an argon atmosphere, the suspension was cooled in an ice-water bath and a solution of (R)-2-(((2,6-dichloropyridin-3-yl)methyl)(methyl)amino)-1-phenylethanol (594 mg, 1.91 mmol) in THF (8 mL) was added via a syringe. The resulting mixture was stirred for 5 minutes with ice-water bath cooling and then the temperature was raised to room temperature and the stirring was continued for 24 h. The reaction mixture was diluted with ethyl acetate, washed with water, dried over MgSO₄ and evaporated to give 518 mg of the title product (16% Yield).

MS m/z 275, 277 [M+H]⁺.

The title compound was also prepared using the procedure below:

Acetic acid (0.069 mL, 1.20 mmol) was added to a solution of (R)-8-chloro-2-phenyl-2,3,4,5-tetrahydropyrido[3,2-f][1,4]oxazepine (Example 11b, 314 mg, 1.20 mmol) and formaldehyde (37% aqueous, 103 mg, 1.26 mmol) in methanol (5 mL). The solution was stirred at room temperature for 30 min and then NaCNBH₄ (114 mg, 1.81 mmol) was added. The resulting mixture was stirred for 1 h. The reaction was quenched by the addition of saturated aqueous NaHCO₃, the methanol was evaporated at reduced pressure and the aqueous residue was extracted with dichloromethane. The combined organic layers were washed with water, dried over Na₂SO₄ and evaporated to give 308 mg of the title product (93% Yield).

¹H NMR (400 MHz, DMSO-d₆) δ ppm 7.82 (d, 1H) 7.44-7.49 (m, 2H) 7.38-7.44 (m, 2H) 7.34-7.38 (m, 1H) 7.26 (d, 1H) 5.19 (dd, 1H) 3.83-3.90 (m, 1H) 3.75-3.82 (m, 1H) 3.03-3.12 (m, 1H) 2.96-3.03 (m, 1H) 2.36 (s, 3H). MS m/z 275, 277 [M+H]^{+.}

### Example 9b

### (R)-2-(((2,6-dichloropyridin-3-yl)methyl)(methyl)amino)-1-phenylethanol

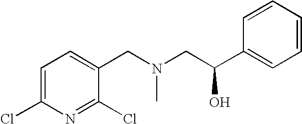

To a solution of (R)-2-((2,6-dichloropyridin-3-yl)methylamino)-1-phenylethanol (565 mg, 1.90 mmol) in THF (8 mL) was added formaldehyde (0.177 mL, 2.38 mmol) and acetic acid (0.027 mL, 0.48 mmol). The resulting mixture was stirred for 40 min prior to the addition of NaCNBH₄ (209 mg, 3.33 mmol). Stirring was continued for another 40 min and then the reaction was quenched by the addition of water. The mixture was extracted with ethyl acetate, the combined organic extracts were washed with water and brine, dried over MgSO₄ and evaporated to give 598 mg of the title product (100% Yield).

¹H NMR (400 MHz, DMSO-d₆) δ ppm 7.84 (d, 1H) 7.48 (d, 1H) 7.25 (m, 5H) 5.16 (d, 1H) 4.73 (m, 1H) 3.63 (s, 2H) 2.61 (dd, 1H) 2.53 (m, 1H) 2.28 (s, 3H).

MS m/z 311, 313 [M+H]⁺369, 371 [M+OAc]⁻

### Example 9c

### (R)-2-((2,6-dichloropyridin-3-yl)methylamino)-1-phenylethanol

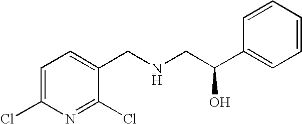

N-Ethyldiisopropylamine (757 μl, 4.34 mmol) was added to a solution of 2,6-dichloro-3-(chloromethyl)pyridine (568 mg, 2.89 mmol) and (R)-2-amino-1-phenylethanol (436 mg, 3.18 mmol) in DMF, the resulting solution was stirred at room temperature for 16 h. The solvent was evaporated at reduced pressure, the residue was dissolved in dichloromethane and the solution was washed with dilute aqueous HCl, water and brine, dried over Na₂SO₄ and evaporated. The compound was recrystallized from diethyl ether to give 568 mg of the title product (66% Yield).

¹H NMR (400 MHz, DMSO-d₆) δ ppm 7.96 (d, 1H) 7.55 (d, 1H) 7.31 (m, 4H) 7.23 (m, 1H) 5.35 (d, 1H) 4.66 (m, 1H) 3.79 (s, 2H) 2.64 (d, 2H).

### Example 9d

### 6-methoxy-5-(4-methyl-1H-imidazol-1-yl)pyridin-2-amine

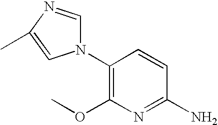

A mixture of 5-bromo-6-methoxypyridin-2-amine (573 mg, 2.82 mmol), 4-methyl-1H-imidazole (324 mg, 3.95 mmol), copper (I) iodide (107 mg, 0.56 mmol) and CS₂CO₃ (1839 mg, 5.64 mmol) in DMF (5 mL) was heated to 140° C. under argon atmosphere in a microwave reactor for 1 h and then at 150° C. for 1 h. The reaction mixture was diluted with dichloromethane and methanol and filtered through a plug of Celite. The solvent was evaporated and the residue was purified by preparative HPLC to give 128 mg of the title product (22% Yield).

¹H NMR (500 MHz, DMSO-d₆) δ ppm 7.54 (d, 1H) 7.33 (d, 1H) 6.92 (t, 1H) 6.19 (s, 2H) 6.05 (d, 1H) 3.77 (s, 3H) 2.11 (m, 3H).

MS m/z 205 [M+H]⁺203 [M−H]⁻

### Example 10

### (R)—N-(3-methoxy-4-(4-methyl-1H-imidazol-1-yl)phenyl)-4-methyl-2-phenyl-2,3,4,5-tetrahydropyrido[3,2-f][1,4]oxazepin-8-amine

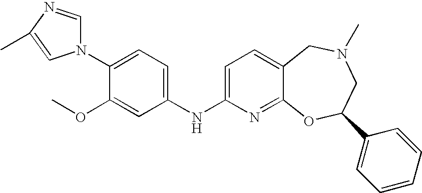

The title compound was prepared in an analogous procedure as described in Example 9 using (R)-8-chloro-4-methyl-2-phenyl-2,3,4,5-tetrahydropyrido[3,2-f][1,4]oxazepine and 3-methoxy-4-(4-methyl-1H-imidazol-1-yl)aniline. The crude product was purified by column chromatography on Silica using gradient elution with an increasing concentration of methanol, from 0 to 10%, in dichloromethane. This was then further purified by preparative chromatography for chiral separation to give 90 mg of the title compound (35% Yield).

¹H NMR (500 MHz, DMSO-d₆) δ ppm 9.25 (s, 1H) 7.67 (s, 1H) 7.60 (m, 1H) 7.54 (d, 1H) 7.49 (m, 2H) 7.40 (m, 2H) 7.33 (m, 1H) 7.17 (m, 2H) 6.99 (s, 1H) 6.60 (d, 1H) 5.10 (m, 1H) 3.69 (m, 5H) 3.05 (m, 1H) 2.98 (m, 1H) 2.36 (s, 3H) 2.12 (s, 3H).

MS m/z 442 [M+H]⁺440 [M−H]⁻.

### Example 11

### (R)-1-(8-(6-methoxy-5-(4-methyl-1H-imidazol-1-yl)pyridin-2-ylamino)-2-phenyl-2,3-dihydropyrido[3,2-f][1,4]oxazepin-4(5H)-yl)ethanone

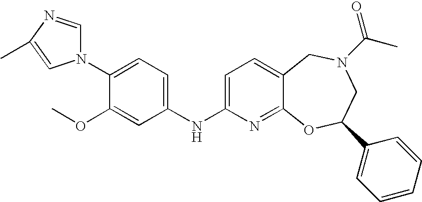

The title compound was prepared in an analogous procedure as described in Example 9 using (R)-1-(8-chloro-2-phenyl-2,3-dihydropyrido[3,2-f][1,4]oxazepin-4(5H)-yl)ethanone as starting material (23% Yield).

Mixture of rotamers: ¹H NMR (500 MHz, DMSO-d₆) d ppm 9.93 (s) 9.88 (s) 7.79 (d) 7.62-7.72 (m) 7.58 (m) 7.51 (m) 7.45 (m) 7.36-7.41 (m) 7.06 (m) 7.02 (app dd) 5.40 (m) 5.24 (m) 4.86 (m) 4.83 (s) 4.54 (d) 4.40 (d) 4.07 (m) 3.92-4.0 (m) 3.78-3.88 (m) 2.13 (s) 2.06 (app d). Total number of protons in spectrum: 26.

Ratio rotamer 1:rotamer 2: 1:1.

MS m/z 471 [M+H]⁺469 [M−H]⁻.

### Example 11a

### (R)-1-(8-chloro-2-phenyl-2,3-dihydropyrido[3,2-f][1,4]oxazepin-4(5H)-yl)ethanone

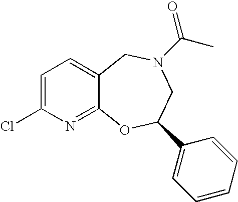

Acetic anhydride (245 mg, 2.40 mmol) was added to a solution of (R)-8-chloro-2-phenyl-2,3,4,5-tetrahydropyrido[3,2-f][1,4]oxazepine (569 mg, 2.18 mmol) in dichloromethane (5 mL) and the resulting solution was allowed to react at room temperature for 30 min. The mixture was diluted with dichloromethane, washed with saturated aqueous NaHCO₃, dried over Na₂SO₄ and concentrated to give 646 mg of the title product as a solid (98% Yield).

MS m/z 303, 305 [M+H]⁺301, 303 [M−H]⁻.

### Example 11b

### (R)-8-chloro-2-phenyl-2,3,4,5-tetrahydropyrido[3,2-f][1,4]oxazepine

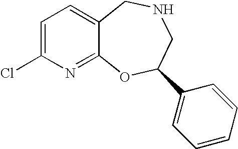

Sodium hydride (0.306 g, 12.76 mmol) was added in portions to a stirred solution of (R)-2-((2,6-dichloropyridin-3-yl)methylamino)-1-phenylethanol (Example 9c, 2.528 g, 8.51 mmol) in THF (10 mL) at 0° C. After 5 minutes the reaction mixture was stirred at room temperature for 2 hours and then heated to 50° C. for 1 hour. The reaction was stirred at room temperature overnight, 0.3 eq NaH (dispensed in mineral oil) added and stirred at room temperature over the weekend. The solvent was evaporated and the residue was partitioned between dichloromethane and saturated NaHCO₃ (aq). The water layer was extracted twice with dichloromethane. The combined organic phase was dried over MgSO₄ and concentrated. The crude product was purified by silica flash chromatography using a gradient of methanol (0 to 5%) in dichloromethane giving 1.716 g (77% Yield).

¹H NMR (600 MHz, DMSO-d₆) δ ppm 7.75 (d, 1H) 7.38-7.47 (m, 4H) 7.34 (t, 1H) 7.21 (d, 1H) 5.02 (dd, 1H) 4.03 (d, 1H) 3.79 (d, 1H) 3.20 (dd, 1H) 3.07 (dd, 1H)

MS m/z 260 [M+H]⁺.

### Example 12

### (R)-6-(2-fluoroethyl)-N-(3-methoxy-4-(4-methyl-1H-imidazol-1-yl)phenyl)-8-phenyl-5,6,7,8-tetrahydropyrimido[5,4-f][1,4]oxazepin-2-amine

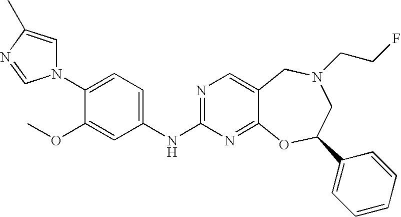

The title compound was prepared in an analogous procedure as described in Example 9 using 3-methoxy-4-(4-methyl-1H-imidazol-1-yl)aniline and (R)-2-chloro-6-(2-fluoroethyl)-8-phenyl-5,6,7,8-tetrahydropyrimido[5,4-f][1,4]oxazepine as starting material (15% Yield).

¹H NMR (500 MHz, DMSO-d₆) δ ppm 9.78 (d, 1H) 8.34 (d, 1H) 7.67-7.74 (m, 1H) 7.60-7.67 (m, 1H) 7.45-7.53 (m, 2H) 7.38-7.45 (m, 3H) 7.32-7.38 (m, 1H) 7.19-7.24 (m, 1H) 6.99-7.04 (m, 1H) 5.45-5.52 (m, 1H) 4.58-4.66 (m, 1H) 4.53 (d, 1H) 4.05 (d, 1H) 3.79 (d, 1H) 3.75 (d, 3H) 3.20-3.27 (m, 1H) 3.13-3.19 (m, 1H) 2.93-2.98 (m, 1H) 2.87-2.93 (m, 1H) 2.13 (s, 3H)

MS (ES+) m/z 475 [M+H]⁺.

### Example 12a

### (R)-2-chloro-6-(2-fluoroethyl)-8-phenyl-5,6,7,8-tetrahydropyrimido[5,4-f][1,4]oxazepine

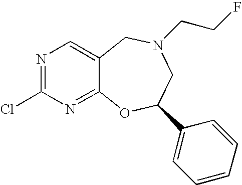

A solution of (R)-2-chloro-8-phenyl-5,6,7,8-tetrahydropyrimido[5,4-f][1,4]oxazepine (0.107 g, 0.41 mmol), 1-bromo-2-fluoroethane (0.091 mL, 1.23 mmol) and n-ethyldiisopropylamine (0.086 mL, 0.49 mmol) in DMF (2 mL) was stirred at room temperature overnight. The solvent was evaporated and the residue was partitioned between water and dichloromethane. The organic layer was dried over MgSO₄ and to evaporated to give 0.129 g of the title compound (103% Yield).

MS (ES+) m/z 308 [M+H]⁺.

### Example 12b

### (R)-2-chloro-8-phenyl-5,6,7,8-tetrahydropyrimido[5,4-f][1,4]oxazepine

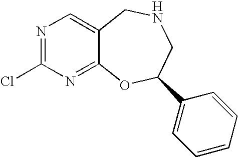

A solution of 2,4-dichloro-5-(iodomethyl)pyrimidine (1.428 g, 4.94 mmol) in THF (2 mL) was cooled to −78° C. (R)-2-amino-1-phenylethanol (0.616 g, 4.49 mmol) in THF (2 mL) was added dropwise. The reaction mixture was stirred at −78° C. for 10 minutes. The reaction mixture was cooled in an ice water bath and sodium hydride (60% dispensed in mineral oil, 0.216 g, 8.99 mmol) was added. After 10 minutes, 2 eq of sodium hydride (60% dispensed in mineral oil, 0.216 g, 8.99 mmol) were added and the reaction mixture stirred at room temperature for 30 minutes. Water (3 mL) was added and the solvent was evaporated. The residue was partitioned between saturated NaHCO₃ (aq) and dichloromethane. The water layer was extracted twice with dichloromethane. The combined organic layers was dried (MgSO₄) and concentrated. The crude product was purified by silica flash chromatography using a gradient of methanol (0 to 3%) in dichloromethane giving 0.193 g of the title compound (16% Yield).

¹H NMR (500 MHz, DMSO-d₆) δ ppm 8.48 (d, 1H) 7.40-7.49 (m, 5H) 5.60-5.67 (m, 1H) 4.20 (d, 1H) 3.77 (d, 1H) 3.20 (br s, 1H) 3.14-3.18 (m, 1H) 3.04 (br s, 1H).

MS (ES+) m/z 262 [M+H]⁺.

### Example 13

### (R)—N-(3-methoxy-4-(4-methyl-1H-imidazol-1-yl)phenyl)-6-methyl-8-phenyl-5,6,7,8-tetrahydropyrimido[5,4-f][1,4]oxazepin-2-amine

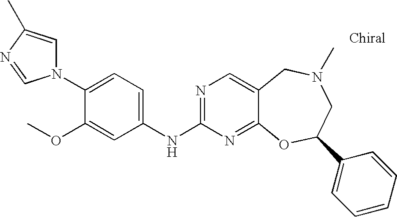

The title compound was prepared in an analogous procedure as described in Example 9 using 3-methoxy-4-(4-methyl-1H-imidazol-1-yl)aniline and (R)-2-chloro-6-methyl-8-phenyl-5,6,7,8-tetrahydropyrimido[5,4-f][1,4]oxazepine as starting material (7% Yield).

¹H NMR (500 MHz, DMSO-d₆) δ ppm 9.77 (s, 1H) 8.34 (s, 1H) 7.69 (d, 1H) 7.64 (d, 1H) 7.46-7.54 (m, 2H) 7.38-7.46 (m, 3H) 7.33-7.38 (m, 1H) 7.19-7.25 (m, 1H) 6.99-7.06 (m, 1H) 5.41 (d, 1H) 3.87 (d, 1H) 3.75 (d, 3H) 3.61 (d, 1H) 3.04-3.13 (m, 1H) 3.01 (s, 1H) 2.40 (d, 3H) 2.13 (s, 3H).

MS (ES+) m/z 443 [M+H]⁺.

### Example 13a

### (R)-2-chloro-6-methyl-8-phenyl-5,6,7,8-tetrahydropyrimido[5,4-f][1,4]oxazepine

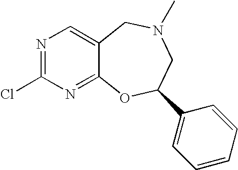

A mixture of formaldehyde (8.51 μL, 0.31 mmol), (R)-2-chloro-8-phenyl-5,6,7,8-tetrahydropyrimido[5,4-f][1,4]oxazepine (0.077 g, 0.29 mmol) and acetic acid (8.42 μL, 0.15 mmol) in methanol (2 mL) was stirred at 0° C. for 15 minutes. A spoon of polymer bound cyanoborohydride was added. After 5 minutes the ice water bath was removed and the reaction was stirred at room temperature for 3 hours. The reaction mixture was filtrated and the filtrate was concentrated giving 0.082 of the title compound (101% Yield)

MS (ES+) m/z 276 [M+H]⁺.

### Example 14

### 2-(8-(3-methoxy-4-(4-methyl-1H-imidazol-1-yl)phenylamino)-2-phenyl-2,3-dihydropyrido[3,2-f][1,4]oxazepin-4(5H)-yl)acetonitrile

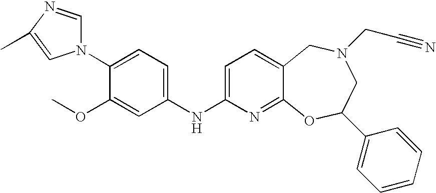

The title compound was prepared in an analogous procedure as described in Example 9 using 2-(8-chloro-2-phenyl-2,3-dihydropyrido[3,2-f][1,4]oxazepin-4(5H)-yl)acetonitrile and 3-methoxy-4-(4-methyl-1H-imidazol-1-yl)aniline as starting material (9% Yield).

¹H NMR (500 MHz, DMSO-d₆) δ ppm 9.31 (s, 1H) 7.66 (d, 1H) 7.61 (d, 1H) 7.53 (m, 2H) 7.51 (m, 1H) 7.42 (m, 2H) 7.35 (m, 1H) 7.18 (m, 2H) 6.99 (t, 1H) 6.62 (d, 1H) 5.14 (m, 1H) 3.92 (s, 2H) 3.85 (d, 1H) 3.77 (d, 1H) 3.70 (s, 3H) 3.20 (m, 1H) 3.08 (dd, 1H) 2.12 (d, 3H).

MS m/z 467 [M+H]⁺465 [M−H]⁻.

### Example 14a

### 2-(8-chloro-2-phenyl-2,3-dihydropyrido[3,2-f][1,4]oxazepin-4(5H)-yl)acetonitrile

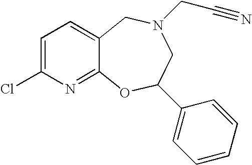

2-Bromoacetonitrile (0.025 mL, 0.36 mmol) was added to a solution of 8-chloro-2-phenyl-2,3,4,5-tetrahydropyrido[3,2-f][1,4]oxazepine (85 mg, 0.33 mmol) and TEA (0.050 mL, 0.36 mmol) in THF (1 mL). The mixture was stirred at room temperature for 16 h and then diluted with ethyl acetate and washed with water and brine. The solution was dried over Na₂SO₄ and evaporated to give 93 mg of the title product (95% Yield).

MS m/z 300, 302 [M+H]⁺298, 300 [M−H]⁻.

### Example 14b

### 8-chloro-2-phenyl-2,3,4,5-tetrahydropyrido[3,2-f][1,4]oxazepine

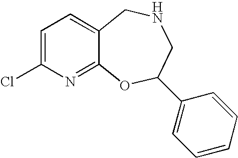

Sodium hydride (0.195 g, 8.14 mmol) was added in portions to a solution of 2-((2,6-dichloropyridin-3-yl)methylamino)-1-phenylethanol (1.613 g, 5.43 mmol) in THF (15 mL) at 0° C. After 5 minutes the reaction mixture was allowed to reach room temperature and stirred over the weekend. The solvent was evaporated and the residue was partitioned between ethyl acetate and saturated NaHCO₃ (aq.). The organic layer was further washed with brine and water, dried (MgSO₄) and concentrated. The crude product was purified by silica flash chromatography using a gradient of methanol (0 to 3%) in dichloromethane giving 1.084 g of the title compound (77% Yield).

¹H NMR (500 MHz, DMSO-d₆) δ ppm 7.75 (d, 1H) 7.42 (m, 4H) 7.33 (m, 1H) 7.21 (d, 1H) 5.02 (dd, 1H) 4.03 (d, 1H) 3.79 (m, 1H) 3.20 (m, 1H) 3.06 (m, 1H).

MS (ES+) m/z 261 [M+H]⁺.

### Example 14c

### 2-((2,6-dichloropyridin-3-yl)methylamino)-1-phenylethanol

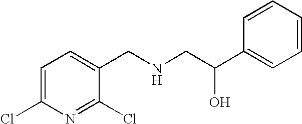

A solution of 2,6-dichloronicotinaldehyde (612 mg, 3.48 mmol) and 2-amino-1-phenylethanol (501 mg, 3.65 mmol) in methanol (5 mL) was stirred at room temperature for 2 h. Acetic acid (0.199 mL, 3.48 mmol) and NaCNBH₄ (437 mg, 6.95 mmol) were added and stirring was continued for an additional 30 minutes. The solvent was evaporated and the residue was partitioned between saturated aqueous NaHCO₃ and dichloromethane. The aqueous phase was extracted with one aliquot of dichlormethane and the combined organic layers were washed with water and brine, dried over Na₂SO₄ and evaporated. The residue was purified by column chromatography on silica using gradient elution with increasing concentration of methanol, from 0 to 5%, in dichloromethane to give 558 mg of the title compound (98.2% Yield).

¹H NMR (400 MHz, DMSO-d₆) δ ppm 7.96 (d, 1H) 7.54 (d, 1H) 7.31 (m, 4H) 7.22 (m, 1H) 5.34 (d, 1H) 4.66 (m, 1H) 3.79 (s, 2H) 2.64 (d, 2H)

MS (ES+) m/z 297, 299 [M+H]⁺355, 357 [M+OAc]⁻.

### Example 15

### 4-(2-fluoroethyl)-N-(3-methoxy-4-(4-methyl-1H-imidazol-1-yl)phenyl)-2-phenyl-2,3,4,5-tetrahydropyrido[3,2-f][1,4]oxazepin-8-amine

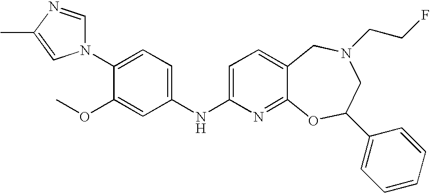

The title compound was prepared in an analogous procedure as described in Example 9 using 3-methoxy-4-(4-methyl-1H-imidazol-1-yl)aniline and 8-chloro-4-(2-fluoroethyl)-2-phenyl-2,3,4,5-tetrahydropyrido[3,2-f][1,4]oxazepine as starting material (32% Yield).

¹H NMR (500 MHz, DMSO-d₆) δ ppm 9.27 (s, 1H) 7.67 (d, 1H) 7.61 (d, 1H) 7.55 (d, 1H) 7.49 (m, 2H) 7.40 (m, 2H) 7.33 (m, 1H) 7.18 (m, 2H) 6.99 (m, 1H) 6.61 (d, 1H) 5.13 (dd, 1H) 4.62 (m, 1H) 4.53 (m, 1H) 3.87 (m, 2H) 3.70 (s, 3H) 3.17 (m, 2H) 2.88 (m, 2H) 2.12 (s, 3H).

MS m/z 474 [M+H]⁺472 [M−H]⁻.

### Example 15a

### 8-chloro-4-(2-fluoroethyl)-2-phenyl-2,3,4,5-tetrahydropyrido[3,2-f][1,4]oxazepine

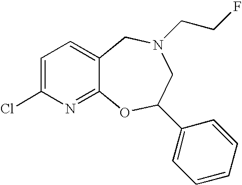

A solution of 8-chloro-2-phenyl-2,3,4,5-tetrahydropyrido[3,2-f][1,4]oxazepine (112 mg, 0.43 mmol), 1-bromo-2-fluoroethane (164 mg, 1.29 mmol) and N-ethyldiisopropylamine (0.090 mL, 0.52 mmol) in DMF (2 mL) was stirred at 70° C. for 16 h. The solvent was evaporated and the residue was partitioned between water and dichloromethane. The organic layer was dried over Na₂SO₄ and evaporated to give 122 mg of the title product (93% Yield).

MS m/z 307, 309 [M+H]⁺305, 307 [M−H]⁻

### Example 16

### N-(3-fluoro-4-(4-methyl-1H-imidazol-1-yl)phenyl)-4-methyl-2-phenyl-2,3,4,5-tetrahydropyrido[3,2-f][1,4]oxazepin-8-amine

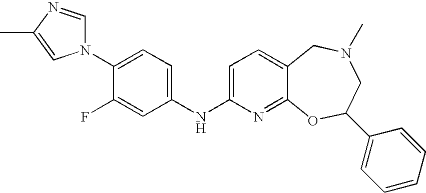

The title compound was prepared in an analogous procedure as described in Example 9 using 8-chloro-4-methyl-2-phenyl-2,3,4,5-tetrahydropyrido[3,2-f][1,4]oxazepine and 3-fluoro-4-(4-methyl-1H-imidazol-1-yl)aniline as starting material (17% Yield).

¹H NMR (600 MHz, DMSO-d₆) δ ppm 9.45 (s, 1H) 7.84 (dd, 1H) 7.74 (br s, 1H) 7.57 (d, 1H) 7.49 (d, 2H) 7.41 (m, 3H) 7.32-7.37 (m, 2H) 7.12 (br s, 1H) 6.61 (d, 1H) 5.11 (m, 1H) 3.70 (m, 2H) 2.99 (d, 2H) 2.36 (s, 3H) 2.13 (s, 3H).

MS m/z 430 [M+H]⁺428 [M−H]⁻.

### Example 17

### 1-(8-(3-methoxy-4-(4-methyl-1H-imidazol-1-yl)phenylamino)-2-phenyl-2,3-dihydropyrido[3,2-f][1,4]oxazepin-4(5H)-yl)ethanone

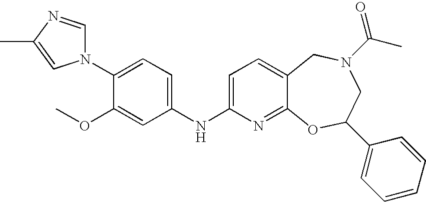

The title compound was prepared in an analogous procedure as described in Example 9 using 1-(8-chloro-2-phenyl-2,3-dihydropyrido[3,2-f][1,4]oxazepin-4(5H)-yl)ethanone and 3-methoxy-4-(4-methyl-1H-imidazol-1-yl)aniline as starting material (92% Yield).

Mixture of rotamers: ¹H NMR (500 MHz) δ 9.30 (d) 7.56-7.72 (m) 7.53 (m) 7.43-7.46 (m) 7.37-7.40 (m) 7.13-7.18 (m) 6.99 (s) 6.60 (dd) 5.39 (m) 5.22 (m) 4.79-4.82 (m) 4.52 (d) 4.38 (d) 4.12 (m) 4.00 (m) 3.78-3.86 (m) 3.72 (d) 2.12 (s) 2.06 (s). Total no of protons in spectrum: 27

Ratio rotamer 1:rotamer 2: 1:1

MS m/z 470 [M+H]⁺468 [M−H]⁻.

### Example 17a

### 1-(8-chloro-2-phenyl-2,3-dihydropyrido[3,2-f][1,4]oxazepin-4(5H)-yl)ethanone

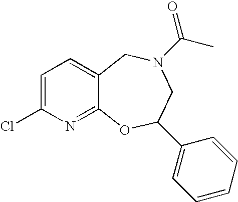

The title compound was prepared in an analogous procedure as described in Example 11a using 8-chloro-2-phenyl-2,3,4,5-tetrahydropyrido[3,2-f][1,4]oxazepine as starting material (95% Yield).

MS m/z 303 [M+H]⁺.

### Example 18

### N-(4-(1H-imidazol-1-yl)phenyl)-4-methyl-2-phenyl-2,3,4,5-tetrahydropyrido[3,2-f][1,4]oxazepin-8-amine

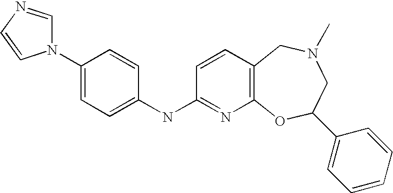

The title compound was prepared in an analogous procedure as described in Example 9 using 4-(1H-imidazol-1-yl)aniline and 8-chloro-4-methyl-2-phenyl-2,3,4,5-tetrahydropyrido[3,2-f][1,4]oxazepine as starting material (41% Yield).

¹H NMR (500 MHz, DMSO-d₆) δ ppm 9.19 (s, 1H) 8.10 (s, 1H) 7.68-7.70 (m, 2H) 7.61 (s, 1H) 7.53 (d, 1H) 7.45-7.51 (m, 4H) 7.40-7.43 (m, 2H) 7.30-7.38 (m, 1H) 7.04 (s, 1H) 6.58 (d, 1H) 5.07-5.09 (m, 1H) 3.61-3.75 (m, 2H) 2.96-2.97 (m, 2H) 2.35 (s, 3H).

MS (ES+) m/z 398 [M+H]⁺.

### Example 19

### N-(3-methoxy-4-(4-methyl-1H-imidazol-1-yl)phenyl)-4-methyl-2-(pyridin-3-yl)-2,3,4,5-tetrahydropyrido[3,2-f][1,4]oxazepin-8-amine

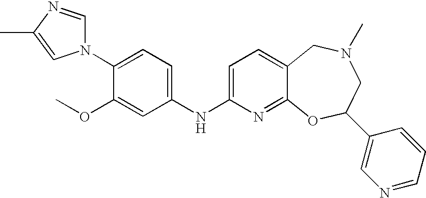

The title compound was prepared in an analogous procedure as described in Example 9 using 3-methoxy-4-(4-methyl-1H-imidazol-1-yl)aniline and 8-chloro-4-methyl-2-(pyridin-3-yl)-2,3,4,5-tetrahydropyrido[3,2-f][1,4]oxazepine as starting material (41% Yield). ¹H NMR (500 MHz, DMSO-d₆) δ ppm 9.29 (s, 1H) 8.71 (d, 1H) 8.54 (dd, 1H) 7.92 (dt, 1H) 7.68 (s, 1H) 7.61 (s, 1H) 7.55 (d, 1H) 7.43 (dd, 1H) 7.12-7.20 (m, 2H) 6.99 (s, 1H) 6.62 (d, 1H) 5.21 (dd, 1H) 3.61-3.80 (m, 5H) 2.99-3.11 (m, 2H) 2.37 (s, 4H) 2.12 (s, 3H).

MS (ES+) m/z 443 [M+H]⁺.

### Example 19a

### 8-chloro-4-methyl-2-(pyridin-3-yl)-2,3,4,5-tetrahydropyrido[3,2-f][1,4]oxazepine

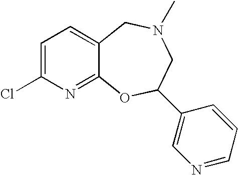

is A reaction mixture of 8-chloro-2-(pyridin-3-yl)-2,3,4,5-tetrahydropyrido[3,2-f][1,4]oxazepine (0.212 g, 0.81 mmol), formaldehyde (0.022 ml, 0.81 mmol) and acetic acid (4.64 μl, 0.08 mmol) in methanol (2 mL) was stirred at room temperature for 20 minutes. Cyanoborohydride, polymer supported (0.648 g, 1.62 mmol) was added and the mixture was stirred for 5 h. The reaction mixture was filtrated and the filtrate was concentrated. The crude product was purified by silica flash chromatography using a gradient of methanol (0 to 5%) in dichloromethane giving 0.069 g of the title product (31% Yield)

¹H NMR (600 MHz, DMSO-d₆) δ ppm 8.68 (s, 1H) 8.53-8.60 (m, 1H) 7.90 (d, 1H) 7.83 (d, 1H) 7.45 (dd, 1H) 7.28 (d, 1H) 5.25-5.34 (m, 1H) 3.79-3.89 (m, 2H) 3.10-3.16 (m, 1H) 3.02-3.07 (m, 1H) 2.38 (s, 3H).

MS (ES+) m/z 276 [M+H]⁺.

### Example 19b

### 8-chloro-2-(pyridin-3-yl)-2,3,4,5-tetrahydropyrido[3,2-f][1,4]oxazepine

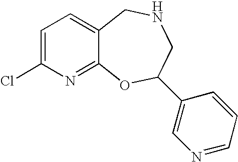

Sodium tert-butoxide (0.149 g, 1.55 mmol) was added in portions to a stirred solution of 2-((2,6-dichloropyridin-3-yl)methylamino)-1-(pyridin-3-yl)ethanol (0.308 g, 1.03 mmol) i THF (4 mL) at 0° C. The reaction mixture was then stirred at room temperature overnight. Added 0.4 eq sodium tert-butoxide and let it continue stir over the weekend. Added 0.5 eq sodium tertbutoxide and let it continue stir overnight. Sodium tertbutoxide (0.3 eq) was added and the reaction stirred for 2 hours. The solvent was evaporated and the residue was suspended in ethyl acetate. The reaction mixture was filtered and the filtrate was dried over MgSO₄ and concentrated giving 0.205 g of the title compound (75% Yield).

¹H NMR (400 MHz, DMSO-d₆) δ ppm 8.65 (d, 1H) 8.56 (dd, 1H) 7.83-7.91 (m, 1H) 7.77 (d, 1H) 7.45 (ddd, 1H) 7.23 (d, 1H) 5.11 (d, 1H) 4.04 (d, 1H) 3.80 (d, 1H) 3.19-3.27 (m, 1H) 3.05-3.19 (m, 1H).

MS (ES+) m/z 262 [M+H]⁺.

### Example 19c

### 2-((2,6-dichloropyridin-3-yl)methylamino)-1-(pyridin-3-yl)ethanol

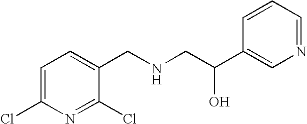

2,6-Dichloronicotinaldehyde (1.376 g, 7.82 mmol), 2-amino-1-(pyridin-3-yl)ethanol (0.982 g, 7.11 mmol) and acetic acid (0.407 ml, 7.11 mmol) in methanol (8 mL) was stirred at 0° C. for 10 minutes. Sodium cyanoborohydride (0.670 g, 10.66 mmol) was added in portions and was stirred at room temperature overnight. Sodium cyanoborohydride (0.300 g) was added and stirred at room temperature over the weekend. Sodium cyanoborohydride (0.300 g) and acetic acid (0.2 mL) were added and the reaction was stirred at 40° C. for 1 h.

The solvent was evaporated and the residue was partitioned between 2% NaOH (aq) and dichloromethane. The organic layer was washed with brine, water and dried (MgSO₄). The solvent was evaporate and the product purified by silica flash chromatography using a gradient of methanol (0 to 3%) in dichloromethane to afford 0.753 g of the title compound (35% Yield).

¹H NMR (600 MHz, DMSO-d₆) δ ppm 8.54 (d, 1H) 8.44 (dd, 1H) 7.94 (d, 1H) 7.73 (dt, 1H) 7.54 (d, 1H) 7.33 (dd, 1H) 5.48 (d, 1H) 4.66-4.77 (m, 1H) 3.79 (s, 2H) 2.70 (d, 2H) 2.41 (br s, 1H).

MS (ES+) m/z 298 [M+H]⁺

### Example 20

### 4-(2-Hydroxyethyl)-8-(3-methoxy-4-(4-methyl-1H-imidazol-1-yl)phenylamino)-2-phenyl-3,4-dihydropyrido[3,2-f][1,4]oxazepin-5(2H)-one

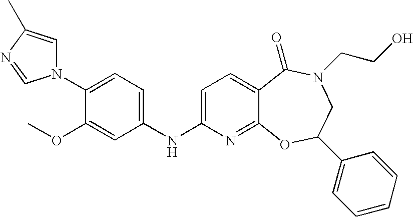

4-(2-(tert-Butyldimethylsilyloxy)ethyl)-8-chloro-2-phenyl-3,4-dihydropyrido[3,2-f][1,4]oxazepin-5(2H)-one (0.120 g, 0.28 mmol), 3-methoxy-4-(4-methyl-1H-imidazol-1-yl)aniline (0.056 g, 0.28 mmol), palladium acetate (6.22 mg, 0.03 mmol), 2-(dicyclohexylphosphino)biphenyl (9.71 mg, 0.03 mmol) and cesium carbonate (0.271 g, 0.83 mmol) were added in a microwave vial. The mixture was capped and flushed with argon. DME (2 mL) was added and the mixture was run in a microwave for 60 minutes at 100° C. The reaction mixture was filtrated through celite and washed with DME. Tetrabutylammonium fluoride (0.097 mL, 0.28 mmol, 1M in THF) was added to the stirred reaction mixture and the mixture was stirred for 1 hr. The reaction was diluted with water and ethyl acetate. The water phase was washed twice with ethyl acetate and the combined organic phase was washed with brine and water, dried (MgSO₄) and concentrated. The product was purified by preparative HPLC to give 0.027 g of the title compound (20% Yield).

¹H NMR (600 MHz, DMSO-d₆) δ ppm 9.71 (s, 1H) 8.02 (d, 1H) 7.86 (d, 1H) 7.66 (d, 1H) 7.55 (d, 2H) 7.41 (t, 2H) 7.33-7.37 (m, 1H) 7.31 (dd, 1H) 7.25 (d, 1H) 7.04 (s, 1H) 6.69 (d, 1H) 4.83 (t, 1H) 3.85-3.93 (m, 1H) 3.79 (dd, 1H) 3.74-3.78 (m, 1H) 3.73 (s, 3H) 3.51-3.64 (m, 2H) 3.25-3.30 (m, 1H) 2.14 (s, 3H).

MS (ES+) m/z 486 [M+H]⁺.

### Example 20a

### 4-(2-(tert-Butyldimethylsilyloxy)ethyl)-8-chloro-2-phenyl-3,4-dihydropyrido[3,2-f][1,4]oxazepin-5(2H)-one

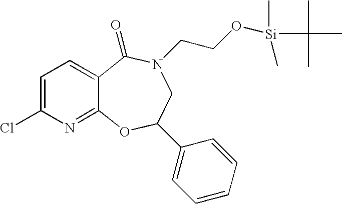

N-(2-(tert-butyldimethylsilyloxy)ethyl)-2,6-dichloro-N-(2-hydroxy-2-phenylethyl)nicotinamide (0.360 g, 0.77 mmol) in dioxan (4 mL) was added dropwise to a stirred suspension of sodium tert-butoxide (0.088 g, 0.92 mmol) in dioxan (2 mL) at 10° C. (ice-water bath). The reaction mixture was stirred at 10-15° C. for 1.5 h and diluted with ethyl acetate and water. The water phase was washed twice with ethyl acetate. The combined organic phase was washed with brine and water, dried (MgSO₄) and concentrated giving 0.327 g of the title compound (98% Yield)

¹H NMR (400 MHz, DMSO-d₆) δ ppm 8.20 (d, 1H) 7.34-7.48 (m, 6H) 5.90 (dd, 1H) 3.90-3.95 (m, 2H) 3.60-3.77 (m, 3H) 3.26-3.33 (m, 1H) 0.81 (s, 9H) 0.00 (d, 6H)

MS m/z 433 [M+H]⁺431 [M−H]⁻.

### Example 20b

### N-(2-(tert-butyldimethylsilyloxy)ethyl)-2,6-dichloro-N-(2-hydroxy-2-phenylethyl)nicotinamide

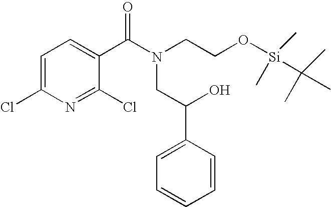

2,6-Dichloronicotinoyl chloride (0.212 g, 1.01 mmol) in THF (5 mL) was added dropwise to a solution of 2-(2-(tert-butyldimethylsilyloxy)ethylamino)-1-phenylethanol (0.297 g, 1.01 mmol) and TEA (0.279 mL, 2.01 mmol) in THF (8 mL) at 0° C. The reaction mixture was slowly allowed to reach room temperature and was stirred overnight. The mixture was diluted with ethyl acetate and washed with water. The organic phase was washed with 0.5 M HCl, saturated NaHCO₃ (aq.) and water, dried over MgSO₄ and concentrated. The product was purified by silica flash chromatography using an increasing concentration of to ethyl acetate (0 to 50%) in heptane to afford 0.361 g of the title compound (77% Yield).

MS (ES+) m/z 469 [M+H]⁺.

### Example 20c

### 2-(2-(tert-butyldimethylsilyloxy)ethylamino)-1-phenylethanol

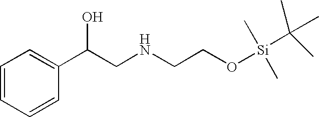

2-Amino-1-phenylethanol (0.984 g, 7.17 mmol) and acetic acid (0.164 ml, 2.87 mmol) were added to a stirred solution of (tert-butyldimethylsilyloxy)acetaldehyde (0.546 ml, 2.87 mmol) in 1,2-dichloroethane (10 mL) at room temperature. The resulting mixture was stirred for 45 minutes and then sodium cyanoborohydride (0.361 g, 5.74 mmol) was added. The mixture was stirred overnight and diluted with dichloromethane, washed with saturated aqueous sodiumhydrogen carbonate solution, water, dried over Na₂SO₄ and concentrated. The product was purified by silica flash chromatography using increasing concentration of methanol (0 to 5%) in dichloromethane to afford 0.311 g of the title compound (37% Yield).

1H NMR (400 MHz, DMSO-d₆) δ ppm 7.25-7.37 (m, 4H) 7.18-7.25 (m, 1H) 5.26 (d, 1H) 4.53-4.66 (m, 1H) 4.11 (q, 1H) 3.61 (t, 2H) 2.56-2.69 (m, 4H) 0.84 (s, 9H) 0.02 (s, 6H).

MS (ES+) m/z 469 [M+H]⁺.

### Example 21

### (S)—N-(3-methoxy-4-(4-methyl-1H-imidazol-1-yl)phenyl)-4-methyl-2-phenyl-2,3,4,5-tetrahydropyrido[3,2-f][1,4]oxazepin-8-amine

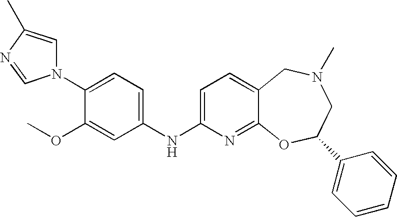

The title compound was prepared in an analogous procedure as described in Example 9 using 3-methoxy-4-(4-methyl-1H-imidazol-1-yl)aniline and (S)-8-chloro-4-methyl-2-phenyl-2,3,4,5-tetrahydropyrido[3,2-f][1,4]oxazepine as starting material (25% Yield).

¹H NMR (600 MHz, DMSO-d₆) δ ppm 9.25 (s, 1H) 7.67 (d, 1H) 7.60 (d, 1H) 7.54 (d, 1H) 7.49 (d, 2H) 7.36-7.44 (m, 2H) 7.29-7.36 (m, 1H) 7.11-7.21 (m, 2H) 6.99 (s, 1H) 6.60 (d, 1H) 5.10 (d, 1H) 3.61-3.77 (m, 5H) 3.02-3.08 (m, 1H) 2.94-3.02 (m, 1H) 2.37 (s, 3H) 2.12 (d, 3H).

MS (ES+) m/z 442 [M+H]⁺.

### Example 21a

### (S)-8-chloro-4-methyl-2-phenyl-2,3,4,5-tetrahydropyrido[3,2-f][1,4]oxazepine

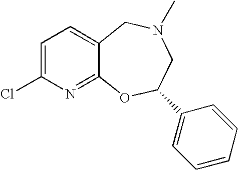

The title compound was prepared in an analogous procedure as described in Example 14b using (S)-2-(((2,6-dichloropyridin-3-yl)methyl)(methyl)amino)-1-phenylethanol as starting material (76% Yield).

¹H NMR (600 MHz, DMSO-d₆) δ ppm 7.81-7.82 (m, 1H) 7.44-7.48 (m, 2H) 7.40-7.42 (m, 2H) 7.34-7.36 (m, 1H) 7.25-7.26 (m, 1H) 5.18-5.20 (m, 1H) 3.77-3.88 (m, 2H) 2.99-3.09 (m, 2H) 2.37 (s, 3H).

MS (ES+) m/z 275 [M+H]⁺.

### Example 21b

### (S)-2-(((2,6-dichloropyridin-3-yl)methyl)(methyl)amino)-1-phenylethanol

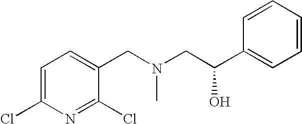

Sodium cyanoborohydride (0.089 g, 1.42 mmol) was added to stirred solution of (S)-2-((2,6-dichloropyridin-3-yl)methylamino)-1-phenylethanol (0.421 g, 1.42 mmol) in anhydrous methanol (3 mL) at room temperature. The mixture was stirred for 10 minutes before formaldehyde (0.041 mL, 1.49 mmol) was added dropwise followed by acetic acid (0.162 mL, 2.83 mmol). The reaction mixture was stirred for 1 h. The solvent was evaporated and the residue was partitioned between ethyl acetate and 0.5 M HCl. The water phase was extracted with ethyl acetate. The combined organic layers was washed with brine, water, dried (MgSO₄) and concentrated. The crude product was purified by silica flash chromatography using an increasing concentration of methanol in ethyl acetate (0 to 3%) and then with methanol (4%) in dichloromethane giving 0.313 g of the title compound (71% Yield).

¹H NMR (600 MHz, DMSO-d₆) δ ppm 7.84 (d, 1H) 7.47 (d, 1H) 7.27-7.33 (m, 4H) 7.23 (t, 1H) 5.13 (d, 1H) 4.65-4.81 (m, 1H) 3.64 (s, 2H) 2.59-2.65 (m, 1H) 2.51-2.55 (m, 1H) 2.28 (s, 3H).

MS (ES+) m/z 311 [M+H]⁺.

### Example 21c

### (S)-2-((2,6-dichloropyridin-3-yl)methylamino)-1-phenylethanol

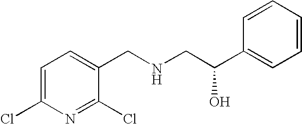

The title compound was prepared in an analogous procedure as described in Example 7b using (S)-(−)-2-Amino-1-phenylethanol as starting material. The crude product was purified by silica flash chromatography using first an increasing concentration of methanol in dichloromethane (0 to 3%) then an increasing concentration of ethyl acetate in heptane (0 to 100%). The product was isolated in 55% yield.

¹H NMR (600 MHz, DMSO-d₆) δ ppm 7.96 (d, 1H) 7.54 (d, 1H) 7.26-7.39 (m, 4H) 7.16-7.26 (m, 1H) 5.32 (d, 1H) 4.58-4.72 (m, 1H) 3.79 (s, 2H) 2.64 (d, 2H)

MS (ES+) m/z 298 [M+H]⁺.

### Example 22

### 1-(8-(3-Methoxy-4-(4-methyl-1H-imidazol-1-yl)phenylamino)-2-(pyridin-3-yl)-2,3-dihydropyrido[3,2-f][1,4]oxazepin-4(5H)-yl)ethanone

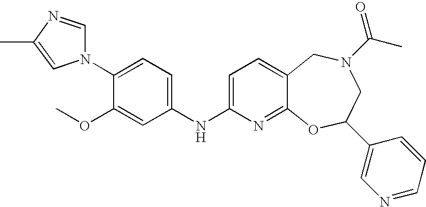

The title compound was prepared in an analogous procedure as described in Example 9 using 3-methoxy-4-(4-methyl-1H-imidazol-1-yl)aniline and 1-(8-chloro-2-(pyridin-3-yl)-2,3-dihydropyrido[3,2-f][1,4]oxazepin-4(5H)-yl)ethanone as starting material (35% Yield).

### Mixture of Rotamers:

¹H NMR (500 MHz, DMSO-d₆) δ ppm 9.36 (s) 9.31 (s) 8.80 (d) 8.74 (d) 8.60 (td) 8.01 (dt) 7.94 (dt) 7.65-7.74 (m) 7.61 (dd) 7.59 (d) 7.49 (dt) 7.12-7.20 (m) 6.97-7.01 (m) 6.62 (dd) 5.49 (dd) 5.34 (dd) 4.81 (d) 4.53 (d) 4.38 (d) 4.13 (dd) 4.04-4.08 (m) 3.82-3.95 (m) 3.68-3.73 (m) 2.12 (s) 2.07 (d)

Total no of protons in spectrum: 26H

Ratio rotamer 1:rotamer 2: 1:1

MS (ES+) m/z 471 [M+H]⁺.

### Example 22a

### 1-(8-Chloro-2-(pyridin-3-yl)-2,3-dihydropyrido[3,2-f][1,4]oxazepin-4(5H)-yl)ethanone

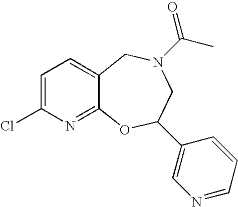

The title compound was prepared in an analogous procedure as described in Example 11a using 8-chloro-2-(pyridin-3-yl)-2,3,4,5-tetrahydropyrido[3,2-f][1,4]oxazepine as starting material and was purified by silica flash chromatography using a gradient of methanol (0 to 5%) in dichloromethane (31% Yield).

¹H NMR (600 MHz, DMSO-d₆) δ ppm 8.73-8.79 (m) 8.70 (d) 8.57-8.65 (m) 7.95-8.02 (m) 7.92 (s) 7.86 (d) 7.47-7.55 (m) 7.30 (d) 7.25 (d) 5.59-5.71 (m) 5.47-5.59 (m) 4.96 (d) 4.82-4.92 (m) 4.68 (d) 4.56 (d) 3.92-4.04 (m) 2.08 (s) 2.03 (s).

Total no of protons in spectrum: 14H

Ratio rotamer 1:rotamer 2: 1:1

MS (ES+) m/z 304 [M+H]⁺.

### Example 23

### 4-methyl-N-(6-(2-methyl-1H-imidazol-1-yl)pyridin-3-yl)-2-phenyl-2,3,4,5-tetrahydropyrido[3,2-f][1,4]oxazepin-8-amine

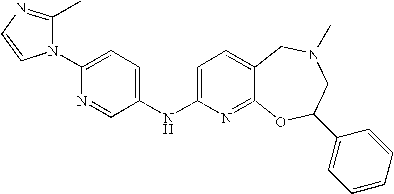

The title compound was prepared in an analogous procedure as described in Example 9 using 8-chloro-4-methyl-2-phenyl-2,3,4,5-tetrahydropyrido[3,2-f][1,4]oxazepine and 6-(2-methyl-1H-imidazol-1-yl)pyridin-3-amine as starting material (25% Yield).

¹H NMR (600 MHz, DMSO-d₆) δ ppm 9.46 (s, 1H) 8.71 (d, 1H) 8.25 (dd, 1H) 7.58 (d, 1H) 7.48 (m, 2H) 7.45 (d, 1H) 7.41 (m, 2H) 7.39 (d, 1H) 7.34 (m, 1H) 6.85 (d, 1H) 6.63 (d, 1H) 5.11 (dd, 1H) 3.70 (m, 2H) 2.98 (m, 2H) 2.39 (s, 3H) 2.36 (s, 3H).

MS m/z 413 [M+H]⁺411 [M−H]⁻.

### Example 24

### 4-(2-hydroxyethyl)-8-(4-(2-methyl-1H-imidazol-1-yl)phenylamino)-2-phenyl-3,4-dihydropyrido[3,2-f][1,4]oxazepin-5(2H)-one

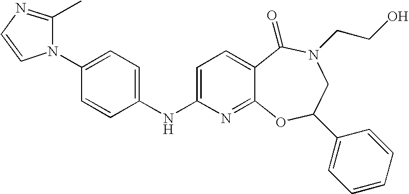

4-(2-(tert-Butyldimethylsilyloxy)ethyl)-8-chloro-2-phenyl-3,4-dihydropyrido[3,2-f][1,4]oxazepin-5(2H)-one (0.104 g, 0.24 mmol), 4-(2-methyl-1H-imidazol-1-yl)aniline (0.050 g, 0.29 mmol), palladium acetate (5.39 mg, 0.02 mmol) and cesium carbonate (0.235 g, 0.72 mmol) were added in a microwave vial. The mixture was capped and flushed with argon. DME (2 mL) was added and the mixture was run in a microwave for 1 h at 100° C. The reaction mixture was filtrated through celite and washed with DME. Hydrogen chloride (1.0M in diethyl ether, 0.012 mL, 0.24 mmol) was added dropwise to the stirred solution and the mixture was stirred at room temperature for two hours. Washed with saturated sodium hydrogencarbonate aqueous solution, brine and water, dried (MgSO₄) and concentrated. The crude was dissolved in DME (2 mL) and tetrabutylammonium fluoride (0.084 mL, 0.24 mmol, 1.0M in THF) was added. The reaction mixture was stirred at room temperature for 1.5 h. Diluted with ethyl acetate and water. The water phase was washed twice with ethyl acetate. The combined organic phase was washed with brine and water, dried (MgSO₄) and concentrated. The crude product was purified by preparative chromatography giving 0.024 g of the title compound (22% Yield)

¹H NMR (600 MHz, DMSO-d₆) δ ppm 9.71 (s, 1H) 8.03 (d, 1H) 7.81-7.91 (m, 2H) 7.54 (d, 2H) 7.43 (t, 2H) 7.30-7.39 (m, 3H) 7.22 (d, 1H) 6.87 (d, 1H) 6.69 (d, 1H) 5.80 (dd, 1H) 4.80 (t, 1H) 3.79-3.93 (m, 2H) 3.73 (dt, 1H) 3.46-3.62 (m, 2H) 3.15-3.23 (m, 1H).

MS (ES+) m/z 456 [M+H]⁺

### Example 25

### 1-cyclopentyl-4-methyl-8-(4-(2-methyl-1H-imidazol-1-yl)phenylamino)-3,4-dihydro-1H-pyrido[2,3-e][1,4]diazepin-5(2H)-one

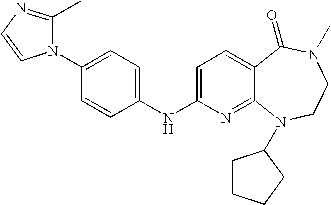

The title compound was prepared in an analogous procedure as described in Example 9 using 8-chloro-1-cyclopentyl-4-methyl-3,4-dihydro-1H-pyrido[2,3-e][1,4]diazepin-5(2H)-one and 4-(2-methyl-1H-imidazol-1-yl)aniline as starting material (46% Yield).

¹H NMR (600 MHz, DMSO-d₆) δ ppm 9.32 (s, 1H) 7.90 (d, 1H) 7.78 (m, 2H) 7.30 (m, 2H) 7.22 (d, 1H) 6.88 (d, 1H) 6.20 (d, 1H) 4.94 (m, 1H) 3.49 (m, 4H) 2.99 (s, 3H) 2.26 (s, 3H) 1.86 (m, 2H) 1.68 (m, 2H) 1.57 (m, 4H).

MS m/z 417 [M+H]⁺415 [M−H]⁻.

### Example 25a

### 8-chloro-1-cyclopentyl-4-methyl-3,4-dihydro-1H-pyrido[2,3-e][1,4]diazepin-5(2H)-one

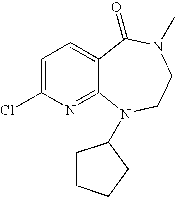

Triethylamine (0.217 mL, 1.56 mmol) was added to a solution of 2,6-dichloro-N-(2-(cyclopentylamino)ethyl)-N-methylnicotinamide (247 mg, 0.78 mmol) in DMF (1 mL). The solution was heated to 80° C. for 66 h. The solvent was evaporated and the residue was partitioned between water and dichloromethane, the aqueous phase was extracted with dichloromethane, the combined extracts were washed with water and brine, dried over Na₂SO₄ and concentrated to give 215 mg of the title compound (98% Yield).

MS m/z 280, 282 [M+H]⁺.

### Example 25b

### 2,6-Dichloro-N-(2-(cyclopentylamino)ethyl)-N-methylnicotinamide

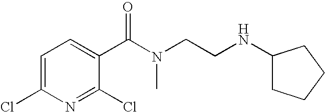

MgSO₄ (50 mg), acetic acid (0.081 mL, 1.41 mmol) and cyclopentanamine (0.347 mL, 3.52 mmol) were added to a solution of 2,6-dichloro-N-methyl-N-(2-oxoethyl)nicotinamide (348 mg, 1.41 mmol) in 1,2-dichloroethane (10 mL). The resulting mixture was stirred at room temperature for 45 minutes and then NaCNBH₄ (177 mg, 2.82 mmol) was added. The mixture was stirred for 6 h at room temperature, and then diluted with dichloromethane, washed with saturated aqueous NaHCO₃, dried over Na₂SO₄ and evaporated. The residue was purified by column chromatography on Silica (12 g), using gradient elution with increasing concentration of methanol (0-10) in dichloromethane, to give 253 mg of the title compound (57% Yield).

MS m/z 316, 318, 320 [M+H]⁺.

### Example 25c

### 2,6-Dichloro-N-methyl-N-(2-oxoethyl)nicotinamide

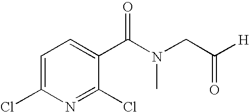

2,6-Dichloro-N-(2,2-dimethoxyethyl)-N-methylnicotinamide (478 mg) was dissolved in chloroform (10 mL), to this was added TFA (2.5 mL) and water (2.5 mL). The mixture was stirred at room temperature over night. Chloroform and most of the TFA was evaporated at reduced pressure. The aqueous residue was made basic by the addition of Na₂CO₃ (s) and extracted with dichloromethane. The combined extracts were dried over Na₂SO₄, and evaporated to give 361 mg of the title compound (80% Yield).

MS m/z 247, 249 [M+H]⁺.

### Example 25d

### 2,6-dichloro-N-(2,2-dimethoxyethyl)-N-methylnicotinamide

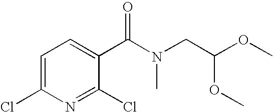

2,6-Dichloronicotinoyl chloride (1.868 g, 8.88 mmol) and TEA (3.71 mL, 26.63 mmol) were dissolved in dichloromethane (10 mL) the solution was cooled in an ice-water bath and a solution of 2,2-dimethoxy-N-methylethanamine (1.269 g, 10.65 mmol) in dichloromethane (10 mL) was added slowly. The resulting mixture was stirred at 0° C. for 30 minutes and then the temperature was allowed to come to room temperature for 30 minutes. The mixture was diluted with dichloromethane, washed with dilute HCl and saturated aqueous NaHCO₃, dried over Na₂SO₄ and evaporated to give 2.479 g of the title compound (95% Yield).

MS m/z 261, 263, 265 [M−OCH3]⁺293, 295 [M+H]⁺

### Example 26

### (R)-2-(8-(4-(6,7-Dihydro-5H-pyrrolo[2,1-c][1,2,4]triazol-3-yl)phenylamino)-2-phenyl-2,3-dihydropyrido[3,2-f][1,4]oxazepin-4(5H)-yl)ethanol

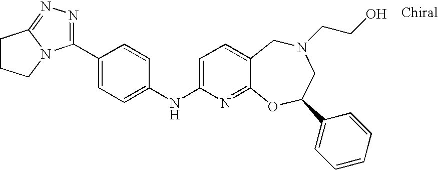

4-(6,7-Dihydro-5H-pyrrolo[2,1-c][1,2,4]triazol-3-yl)aniline (79 mg, 0.39 mmol), (R)-2-(8-chloro-2-phenyl-2,3-dihydropyrido[3,2-f][1,4]oxazepin-4(5H)-yl)ethanol (120 mg, 0.39 mmol), palladium acetate (8.84 mg, 0.04 mmol), 2-(dicyclohexylphosphino)biphenyl (13.80 mg, 0.04 mmol) and cesium carbonate (385 mg, 1.18 mmol) were added to a microwave vial. 1,2-Dimethoxyethane (2 mL) and EtOH (0.500 mL) were added. The reaction mixture was flushed with argon and the mixture was run in a microwave for 60 minutes at 100° C. The crude product was taken up in DCM and the solids were thoroughly washed with DCM. The solvents were evaporated and the crude product was purified by column chromatography on Silica, using an eluent of 0-20% MeOH in DCM with 2% triethylamine giving 39 mg of the title compound (21% Yield).

¹H NMR (400 MHz, DMSO-d₆) δ ppm 9.32 (s, 1H) 7.65-7.73 (m, 4H) 7.56 (d, 1H) 7.47-7.51 (m, 2H) 7.42 (t, 2H) 7.31-7.37 (m, 1H) 6.64 (d, 1H) 5.13 (t, 1H) 4.47 (t, 1H) 4.20 (t, 2H) 3.77-3.90 (m, 2H) 3.54 (q, 2H) 3.12 (d, 2H) 2.83 (t, 2H) 2.60-2.72 (m, 4H). MS m/z 469 [M+H]⁺.

### Example 26a

### (R)-2-(8-chloro-2-phenyl-2,3-dihydropyrido[3,2-f][1,4]oxazepin-4(5H)-yl)ethanol

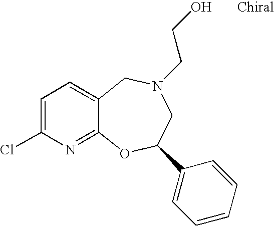

(R)-8-Chloro-2-phenyl-2,3,4,5-tetrahydropyrido[3,2-f][1,4]oxazepine (Example 11b, 2 g, 7.67 mmol), acetic acid (2.189 mL, 38.36 mmol) and 2-hydroxyacetaldehyde (0.461 g, 7.67 mmol) were mixed in methanol (100 mL) at 0° C. and stirred for 15 min. Sodium cyanoborohydride (5.78 g, 92.05 mmol) was added and the reaction was allowed to reach room temp after 5 min and stirred for 2 h. The reaction mixture was concentrated and purified by column chromatography on Silica using 0-8% MeOH in DCM as eluent giving 1.7 g of the title compound (73% Yield). MS m/z 305 [M+H]⁺ 303 [M−H]⁻.

### Example 27

### 2-(2-(1,3-Dimethyl-1H-pyrazol-5-yl)-8-(4-(2-methyl-1H-imidazol-1-yl)phenylamino)-2,3-dihydropyrido[3,2-f][1,4]oxazepin-4(5H)-yl)acetonitrile

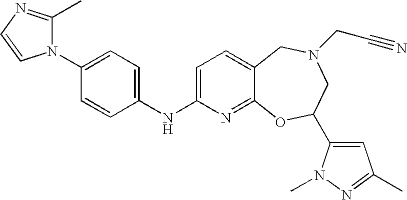

4-(2-Methyl-1H-imidazol-1-yl)aniline (0.121 g, 0.70 mmol), palladium acetate (0.014 g, 0.06 mmol) and cesium carbonate (0.618 g, 1.90 mmol) were added in a microwave vial. The mixture was capped and flushed with argon. 2-(8-chloro-2-(1,3-dimethyl-1H-pyrazol-5-yl)-2,3-dihydropyrido[3,2-f][1,4]oxazepin-4(5H)-yl)acetonitrile (0.201 g, 0.63 mmol) in 1,2-dimethoxyethane (5 mL) was added, the reaction mixture was flushed with argon and the mixture was heated in a microwave oven for 60 min at 100° C. Added 1 eq CsCO₃, and 0.1 eq ligand and palladium acetate, flushed with argon and run in the microwave at 100° C. for 60 min. The reaction mixture was filtrated through Celite and washed with DCM. The filtrate was concentrated and the crude product was purified by preparative chromatography. The product containing fractions were pooled, concentrated and the residue was partitioned between sat. NaHCO₃ (aq) and dichloromethane. The organic layer was separated by a Phase Separator and concentrated giving 0.041 g of the title compound (14% Yield).

¹H NMR (500 MHz, DMSO-d₆) δ ppm 9.38 (s, 1H), 7.79 (m, 2H), 7.53 (d, 1H), 7.33 (m, 2H), 7.21 (d, 1H), 6.87 (d, 1H), 6.63 (d, 1H), 6.16 (s, 1H), 5.17-5.28 (m, 1H), 3.94 (s, 3H), 3.90 (s, 2H), 3.79 (s, 2H), 3.34-3.43 (m, 2H), 2.26 (s, 3H), 2.13 (s, 3H)

MS m/z 455 [M+H]⁺ 453 [M−H]⁻.

### Example 27a

### 2-(8-chloro-2-(1,3-dimethyl-1H-pyrazol-5-yl)-2,3-dihydropyrido[3,2-f][1,4]oxazepin-4(5H)-yl)acetonitrile

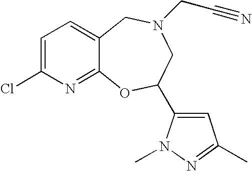

2-Bromoacetonitrile (0.096 mL, 1.39 mmol) was added to a solution of 8-chloro-2-(1,3-dimethyl-1H-pyrazol-5-yl)-2,3,4,5-tetrahydropyrido[3,2-f][1,4]oxazepine (0.351 g, 1.26 mmol) and TEA (0.193 mL, 1.39 mmol) in THF (5 mL) and the reaction mixture is stirred at room temperature for 3.5 h. 2-Bromoacetonitrile (0.04 mL) was added and the reaction mixture was continued stirring at room temperature overnight. The solvent was evaporated and the residue was partitioned between sat. NaHCO₃ (aq) and dichloromethane. The organic layer was washed with water, dried (MgSO₄) and concentrated, giving 0.471 g of the title compound (118% Yield) which was used in the next step without further purification.

¹H NMR (500 MHz, DMSO-d₆) δ ppm 7.80 (d, 1H), 7.30 (d, 1H), 6.17 (s, 1H), 5.39 (d, 1H), 3.99 (d, 1H), 3.94 (s, 2H), 3.86 (s, 1H), 3.82-3.85 (m, 3H), 3.35-3.40 (m, 2H), 2.13 (s, 3H). MS m/z 318 [M+H]⁺.

### Example 27b

### 8-chloro-2-(1,3-dimethyl-1H-pyrazol-5-yl)-2,3,4,5-tetrahydropyrido[3,2-f][1,4]oxazepine

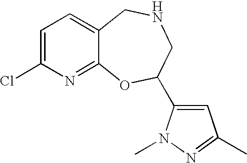

Sodium hydride (0.169 g, 7.06 mmol) was added to a stirred solution of 24(2,6-dichloropyridin-3-yl)methylamino)-1-(1,3-dimethyl-1H-pyrazol-5-yl)ethanol (1.484 g, 4.71 mmol) in THF (10 mL) at 0° C. The reaction mixture was stirred at 0° C. for 5 minutes and then at room temperature overnight. The solvent was evaporated and the crude was partitioned between dichloromethane and sat. NaHCO₃ (aq). The water layer was extracted once with dichloromethane. The combined organic layer was dried (MgSO₄) and concentrated giving 1.478 g of the title compound (113% Yield) which was used in the next step without further purification.

¹H NMR (500 MHz, DMSO-d₆) δ ppm 7.76 (d, 1H), 7.24 (d, 1H), 6.14 (s, 1H), 5.11 (dd, 1H), 3.99 (d, 1H), 3.80-3.85 (m, 3H), 3.77 (d, 1H), 3.34-3.39 (m, 1H), 3.25-3.32 (m, 1H), 2.12 (s, 3H). MS m/z 279 [M+H]⁺.

### Example 27c

### 2-((2,6-dichloropyridin-3-yl)methylamino)-1-(1,3-dimethyl-1H-pyrazol-5-yl)ethanol

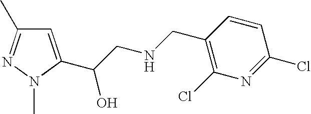

2,6-Dichloronicotinaldehyde (0.964 g, 5.48 mmol), 2-amino-1-(1,3-dimethyl-1H-pyrazol-5-yl)ethanol (0.810 g, 5.22 mmol) and acetic acid (0.149 ml, 2.61 mmol) in methanol (10 mL) was stirred at room temperature for 20 minutes. Methanol (15 mL) was added and cooled to 0° C. Sodium cyanoborohydride (0.394 g, 6.26 mmol) was added and the reaction mixture was stirred at 0° C. for 10 minutes. The solvent was evaporated and the residue was partitioned between dichloromethane and sat. NaHCO₃ (aq). The water layer was extracted twice with dichloromethane and the combined organic layer was dried (MgSO₄) and concentrated. The crude product was purified by silica column chromatography using a gradient of methanol (0 to 5%) in dichloromethane giving 1.50 g of the title compound (91% Yield).

¹H NMR (500 MHz, DMSO-d₆) δ ppm 7.99 (dd, 1H), 7.52-7.63 (m, 1H), 5.90 (s, 1H), 5.37 (dt, 1H), 4.63-4.76 (m, 1H), 3.76-3.86 (m, 2H), 3.65-3.75 (m, 3H), 3.17 (dt, 1H), 2.69-2.87 (m, 2H), 2.01-2.16 (m, 3H). MS m/z 315, 317, 319 [M+H]⁺.

### Example 27d

### 2-amino-1-(1,3-dimethyl-1H-pyrazol-5-yl)ethanol

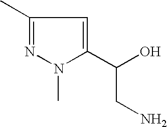

A mixture of 1-(1,3-dimethyl-1H-pyrazol-5-yl)-2-nitroethanol (1.2 g, 6.48 mmol), ammonium formate (2.0 g, 32.4 mmol) and palladium on activated carbon (10%, 300 mg) in anhydrous methanol (40 mL) was stirred at room temperature for 2 h. The reaction mixture was filtered through a pad of Celite and concentrated under reduced pressure. The to residue was purified by flash column chromatography using 5% methanol in dichloromethane to afford 0.8 g of the title compound (79% Yield).

¹H NMR (400 MHz, CHLOROFORM-d) δ ppm 5.92 (s, 1H) 4.60 (dd, 1H) 3.81 (s, 3H) 3.42 (br s, 1H) 2.91-2.98 (m, 2H) 2.18-2.31 (m, 2H) 2.16 (br s, 3H). MS m/z: 156 [M+1]⁺.

### Example 27e

### 1-(1,3-dimethyl-1H-pyrazol-5-yl)-2-nitroethanol

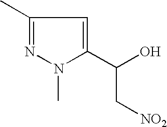

Nitromethane (0.83 g, 13.6 mmol) was added to a mixture of 1,3-dimethyl-1H-pyrazole-5-carbaldehyde (1.0 g, 8.05 mmol) and triethylamine (1.38 g, 13.62 mmol) in anhydrous tetrahydrofuran (5.0 mL) at 0° C. The reaction mixture was stirred at 0° C. for 30 minutes, then allowed to warm to room temperature and concentrated under reduced pressure. The residue was taken up in ethyl acetate (20 mL) and the organic phase was washed with water, dried over Na₂SO₄, filtered and concentrated in vacuo to afford 1.2 g of the title compound (80% Yield).

¹H NMR (400 MHz, CHLOROFORM-d) δ ppm 5.99 (s, 1H) 5.50 (dd, 1H) 4.84-4.73 (m, 1H) 4.68-4.60 (m, 1H) 3.88 (s, 3H) 2.94 (br s, 1H) 2.22 (s, 3H). MS m/z: 186 [M+1]⁺.

### Example 28

### 2-(4-(1H-Imidazol-1-yl)phenoxy)-6-methyl-8-phenyl-7,8-dihydropyrimido[5,4-f][1,4]oxazepin-5(6H)-one

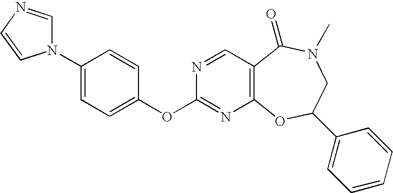

6-Methyl-2-(methylsulfonyl)-8-phenyl-7,8-dihydropyrimido[5,4-f][1,4]oxazepin-5(6H)-one (0.21 g, 0.63 mmol), 4-(1H-imidazol-1-yl)phenol (0.11 g, 0.63 mmol), K₂CO₃ (0.26 g, 1.9 mmol) was added to DMF (5 mL) and the reaction mixture was heated at 75° C. over night. The mixture was filtered and concentrated. The residue was triturated with diethylether. The solid was filtrated and triturated again with a ethylacetate/diethylether mixture. The solid was filtered yielding 0.198 g of the title compound (76% Yield)

¹H NMR (400 MHz, DMSO-d₆) δ ppm 3.02 (s, 3H), 3.76 (d, 1H), 4.24 (dd, 1H), 5.84 (d, 1H), 7.10 (s, 1H), 7.27-7.49 (m, 7H), 7.70 (d, 2H), 7.76 (s, 1H), 8.26 (s, 1H), 8.64 (s, 1H). MS (ES+) m/z: 414 [M+1]⁺.

### Example 28a

### 6-methyl-2-(methylsulfonyl)-8-phenyl-7,8-dihydropyrimido[5,4-f][1,4]oxazepin-5(6H)-one

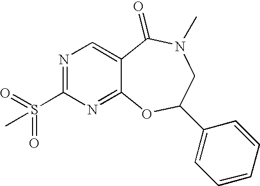

6-Methyl-2-(methylthio)-8-phenyl-7,8-dihydropyrimido[5,4-f][1,4]oxazepin-5(6H)-one (0.500 g, 1.66 mmol) was dissolved in a mixture of carbon tetrachloride (2 mL), acetonitrile (2 mL) and water (2 mL) To this biphasic system was added sodium periodate (1.065 g, 4.98 mmol) while stirring until fully dissolved, followed by ruthenium (III) chloride (0.344 mg, 1.66 μmol). After 1.5 h a bit more water (0.5 mL) was added and the mixture was stirred for another 3 h. The mixture was diluted with water and DCM and the phases were separated. The water layer was extracted with DCM and ethyl acetate. The combined organic layer was washed with brine, dried over anhydrous sodium sulfate, filtered and concentrated giving 0.459 g of the title compound (83% Yield).

MS m/z 334 [M+1]⁺.

### Example 28b

### 6-methyl-2-(methylthio)-8-phenyl-7,8-dihydropyrimido[5,4-f][1,4]oxazepin-5(6H)-one

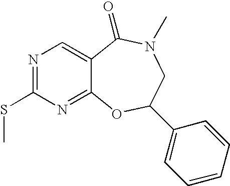

4-(2-(methylamino)-1-phenylethoxy)-2-(methylthio)pyrimidine-5-carboxylic acid (1.4 g, 4.38 mmol) was dissolved in DCM (100 mL). Triethylamine (1.523 mL, 10.96 mmol) and O-(7-azabenzotriazole-1-yl)-N,N,N′,N′-tetramethyluronium hexafluorophosphate (2.500 g, 6.58 mmol) were added and the reaction was stirred at 30° C. over night. The mixture was washed with water and the organics were dried, concentrated and purified by column chromatography on silica using 0-70% EtOAc in heptane as eluent. This gave 1.0 g of the title compound (76% Yield)

MS m/z 302 [M+1]⁺.

### Example 28c

### 4-(2-(methylamino)-1-phenylethoxy)-2-(methylthio)pyrimidine-5-carboxylic acid

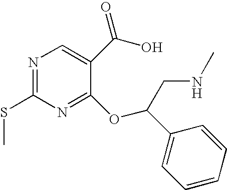

Ethyl 4-(2-(tert-butoxycarbonyl(methyl)amino)-1-phenylethoxy)-2-(methylthio)pyrimidine-5-carboxylate (2.4 g, 5.36 mmol) was dissolved in DCM (25 mL) under argon gas, cooled to 0° C. and trifluoroacetic acid (4.13 mL, 53.63 mmol) was added. The mixture was allowed to reach room temperature and stirred for 3 h. Solvent and TFA was removed in vacuo, the residue was redissolved in THF (25 mL) and cooled to 0° C. Lithium hydroxide (1.541 g, 64.35 mmol) was added and the reaction was stirred for 2 h. The reaction was quenched with citric acid (aq). and extracted with EtOAc. Organics were dried and concentrated to give 1.4 g of the title compound (82% Yield).

MS m/z 320 [M+1]⁺318 [M−1]⁻.

### Example 28d

### ethyl 4-(2-(tert-butoxycarbonylmethyl)amino)-1-phenylethoxy)-2-(methylthio)pyrimidine-5-carboxylate

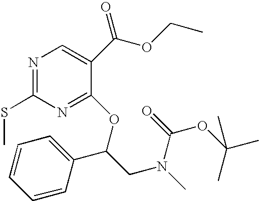

2-(Methylamino)-1-phenylethanol (2 g, 13.23 mmol) was dissolved in DCM (40 mL) and triethylamine (2.212 mL, 15.87 mmol) was added. The mixture was cooled down to 0° C. and treated with di-tert-butyl dicarbonate (3.18 g, 14.55 mmol). The reaction mixture was set under N₂ atmosphere and allowed to warm up to rt. The mixture was diluted with sat NaHCO₃ solution and the phases were separated. The organic layer was washed with brine, dried over sodium sulfate and concentrated. The oil was taken up in ethyl acetate and was washed with water and then with brine. The organic layer was dried over anhydrous sodium sulfate, filtered and concentrated giving 3.29 g of the boc-protected amine. Sodium hydride (dispersed in mineral oil) (0.223 mL, 11.14 mmol) was added to a stirred solution of tert-butyl 2-hydroxy-2-phenylethyl(methyl)carbamate (2.799 g, 11.14 mmol) in anhydrous THF (35 mL) at −78° C. After 5 minutes the reaction mixture was stirred at 0° C. for 1 h, then a solution of ethyl 4-chloro-2-(methylthio)pyrimidine-5-carboxylate (2.160 g, 9.28 mmol) in THF (8 mL) was added drop wise and the reaction mixture was stirred overnight at room temperature. Added 0.5 eq NaH (dispensed in mineral oil). After 5 h, 1 eq of NaH (dispensed in mineral oil) was added and the reaction mixture was stirred overnight. The reaction was quenched with solid ammonium chloride and the solvent was evaporated. The residue was suspended in water and extracted with ethyl acetate. The organic extracts were dried with MgSO₄ and concentrated. The crude product was purified by silica column chromatography using a gradient of ethyl acetate (0 to 50%) in heptane giving 2.95 g of the title compound (70% Yield).

MS m/z 448 [M+H]⁺.

### Example 29

### (R)-4-(4-(2-Hydroxyethyl)-2-phenyl-2,3,4,5-tetrahydropyrido[3,2-f][1,4]oxazepin-8-ylamino)-2-methoxybenzonitrile

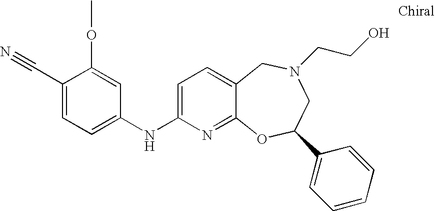

The title compound was prepared in an analogous procedure as described in Example 26 using 4-amino-2-methoxybenzonitrile and (R)-2-(8-chloro-2-phenyl-2,3-dihydropyrido[3,2-f][1,4]oxazepin-4(5H)-yl)ethanol as starting material (13.17% Yield)

¹H NMR (500 MHz, DMSO-d₆) δ ppm 9.67 (s, 1H) 7.77 (d, 1H) 7.61 (d, 1H) 7.49 (d, 3H) 7.40 (t, 2H) 7.30-7.36 (m, 1H) 7.18 (dd, 1H) 6.67 (d, 1H) 5.16 (d, 1H) 4.47 (t, 1H) 3.82-3.90 (m, 2H) 3.78 (s, 3H) 3.55 (q, 2H) 3.18-3.23 (m, 1H) 3.09-3.18 (m, 1H) 2.64 (t, 2H). MS m/z 417 [M+1]⁺.

### Example 30

### (R)-2-(8-(4-(5-methyl-1H-imidazol-1-yl)phenylamino)-2-phenyl-2,3-dihydropyrido[3,2-f][1,4]oxazepin-4(5H)-yl)ethanol

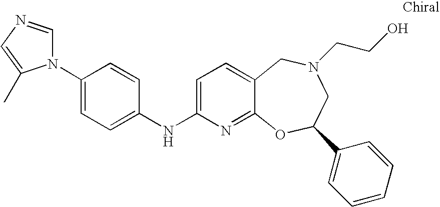

The title compound was prepared in an analogous procedure as described in Example 26 using a mixture of 4-(4-methyl-1H-imidazol-1-yl)aniline (76 mg, 0.44 mmol) and 4-(5-methyl-1H-imidazol-1-yl)aniline together with (R)-2-(8-chloro-2-phenyl-2,3-dihydropyrido[3,2-f][1,4]oxazepin-4(5H)-yl)ethanol as starting material. A Supercritical fluid chromatography (SFC) was performed in order to separate the regioisomers giving 15 mg of the title compound (6% Yield).

¹H NMR (500 MHz, DMSO-d₆) δ ppm 9.27 (s, 1H) 7.69 (m, 2H) 7.64 (s, 1H) 7.55 (d, 1H) 7.47 (d, 2H) 7.40 (t, 2H) 7.33 (t, 1H) 7.26 (m, 2H) 6.76 (s, 1H) 6.62 (d, 1H) 5.12 (t, 1H) 4.47 (br. s., 1H) 3.79-3.89 (m, 2H) 3.54 (q, 2H) 3.12 (d, 2H) 2.64 (t, 2H) 2.09 (s, 3H). MS m/z 417 [M+1]⁺.

### Example 31

### 1-(2-(Isopropoxymethyl)-8-(3-methoxy-4-(4-methyl-1H-imidazol-1-yl)phenylamino)-2,3-dihydropyrido[3,2-f][1,4]oxazepin-4(5H)-yl)ethanone

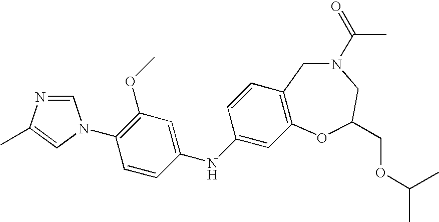

1-(8-Chloro-2-(isopropoxymethyl)-2,3-dihydropyrido[3,2-f][1,4]oxazepin-4(5H)-yl)ethanone (0.169 g, 0.57 mmol), 3-methoxy-4-(4-methyl-1H-imidazol-1-yl)aniline (0.115 g, 0.57 mmol), palladium acetate (0.013 g, 0.06 mmol), 2-(dicyclohexylphosphino)biphenyl (0.020 g, 0.06 mmol) and cesium carbonate (0.553 g, 1.70 mmol) were placed in a microwave vial. The mixture was capped and flushed with argon. 1,2-Dimethoxyethane (5 mL) was added and the mixture was run in a microwave for 60 minutes at 100° C. Ligand (0.1 eq) and palladium acetate (0.1 eq) were added, flushed with argon and run the mixture in the microwave at 100° C. for 60 min. Ligand (0.1 eq) and palladium acetate (0.1 eq) were added, flushed with argon and run the mixture in the microwave at 100° C. for 60 min. The reaction mixture was filtrated through Celite and washed with dichloromethane. The filtrate was concentrated and the crude product was purified by preparative chromatography. The product containing fractions were pooled, concentrated and the residue was partitioned between sat. NaHCO₃ (aq) and dichloromethane. The organic layer was separated by a Phase Separator and concentrated giving 0.039 g of the title compound (15% Yield).

¹H NMR (500 MHz, DMSO-d₆) δ ppm 7.69 (dd, 1H), 7.62-7.65 (m, 1H), 7.21-7.26 (m, 1H), 7.16-7.21 (m, 1H), 7.01 (d, 1H), 6.54 (dd, 1H), 4.32-4.45 (m, 2H), 3.78 (s, 4H), 3.60-3.72 (m, 3H), 3.53-3.59 (m, 1H), 2.13 (d, 3H), 1.99 (d, 3H), 1.11 (dd, 6H). MS m/z 466 [M+H]⁺ 464 [M−H]⁺.

### Example 31a

### 1-(8-chloro-2-(isopropoxymethyl)-2,3-dihydropyrido[3,2-f][1,4]oxazepin-4(5H)-yl)ethanone

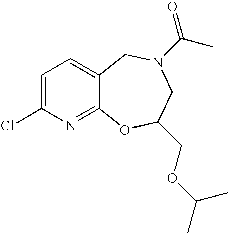

The title compound was prepared in an analogous procedure as described in Example 11a using 8-chloro-2-(isopropoxymethyl)-2,3,4,5-tetrahydropyrido[3,2-f][1,4]oxazepine as starting material (117% Yield).

¹H NMR (500 MHz, DMSO-d₆) δ ppm 7.84-7.86 (d, 0.5H), 7.75-7.77 (d, 0.5H), 7.20-7.21 (d, 0.5H), 7.16-7.17 (d, 0.5H), 4.79-4.83 (d, 1H), 4.59-4.61 (d, 1H), 4.54-4.57 (m, 2H), 3.57-3.69 (m, 4H), 2.00 (s, 1H), 1.95 (s, 1H), 1.91 (s, 1H), 1.11-1.13 (m, 6 H). MS m/z 299 [M+H]⁺.

### Example 31b

### 8-chloro-2-(isopropoxymethyl)-2,3,4,5-tetrahydropyrido[3,2-f][1,4]oxazepine

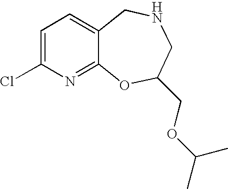

The title compound was prepared in an analogous procedure as described in Example 27b using 1-((2,6-dichloropyridin-3-yl)methylamino)-3-isopropoxypropan-2-ol as starting material (92% Yield). MS m/z 257 [M+H]⁺.

### Example 31c

### 1-((2,6-dichloropyridin-3-yl)methylamino)-3-isopropoxypropan-2-ol

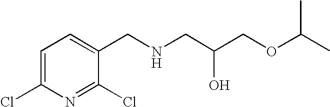

2,6-Dichloronicotinaldehyde (0.694 g, 3.94 mmol), 1-amino-3-isopropoxypropan-2-ol (0.50 g, 3.75 mmol) and acetic acid (0.107 ml, 1.88 mmol) in methanol (10 mL) was stirred at 0° C. for 20 minutes. Sodium cyanoborohydride (0.283 g, 4.50 mmol) was added and the reaction mixture was stirred at 0° C. for 5 minutes then at room temperature overnight. The solvent was evaporated and the residue was partitioned between dichloromethane and sat. NaHCO₃ (aq). The water layer was extracted twice with dichloromethane and the combined organic layer was dried (MgSO₄) and concentrated. The crude product was purified by silica column chromatography using a gradient of methanol (0 to 5%) in dichloromethane giving 0.60 g of the title compound (54.3% Yield).

¹H NMR (500 MHz, DMSO-d₆) δ ppm 8.01 (d, 1H), 7.57 (d, 1H), 4.69 (d, 1H), 3.76 (s, 2H), 3.58-3.67 (m, 1H), 3.50 (dt, 1H), 3.29 (d, 2H), 2.58 (dd, 1H), 2.44 (dd, 1H), 2.29 (br. s., 1H), 1.06 (d, 3H), 1.05 (d, 3H). MS m/z 293, 295, 297 [M+H]⁺.

### Example 32

### (R)-2-(8-(3-Chloro-4-(1H-1,2,4-triazol-1-yl)phenylamino)-2-phenyl-2,3-dihydropyrido[3,2-f][1,4]oxazepin-4(5H)-yl)ethanol

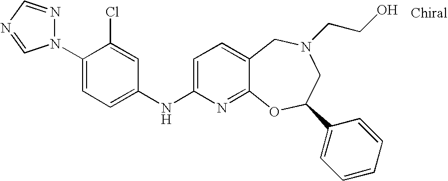

The title compound was prepared in an analogous procedure as described in Example 26 using 3-chloro-4-(1H-1,2,4-triazol-1-yl)aniline and (R)-2-(8-chloro-2-phenyl-2,3-dihydropyrido[3,2-f][1,4]oxazepin-4(5H)-yl)ethanol (Example 27a) as starting material. The crude product was purified on preparative HPLC yielding 16 mg of the title compound (9% Yield)

¹H NMR (500 MHz, DMSO-d₆) δ ppm 9.61 (br. s., 1H) 8.82 (s, 1H) 8.20 (s, 1H) 8.06 (d, 1H) 7.62 (dd, 2H) 7.51 (d, 2H) 7.47 (d, 1H) 7.42 (t, 2H) 7.35 (t, 1H) 6.67 (d, 1H) 5.23 (br. s., 1H) 4.53 (br. s., 1H) 3.92 (br. s., 2H) 3.58 (br. s., 2H) 3.21 (br. s., 2H) 2.70 (br. s., 2H). MS m/z 463 [M+H]⁺ 461 [M−H]⁻.

### Example 33

### 2-(2-(1,3-Dimethyl-1H-pyrazol-5-yl)-8-(3-methoxy-4-(4-methyl-1H-imidazol-1-yl)phenylamino)-2,3-dihydropyrido[3,2-f][1,4]oxazepin-4(5H)-yl)acetonitrile

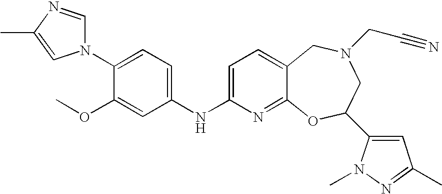

The title compound was prepared in an analogous procedure as described in Example 1 using 3-methoxy-4-(4-methyl-1H-imidazol-1-yl)aniline and 2-(8-chloro-2-(1,3-dimethyl-1H-pyrazol-5-yl)-2,3-dihydropyrido[3,2-f][1,4]oxazepin-4(5H)-yl)acetonitrile (Example 27a) as starting material (17% Yield).

¹H NMR (500 MHz, DMSO-d₆) δ ppm 9.35 (s, 1H), 7.65 (d, 1H), 7.46-7.58 (m, 2H), 7.30-7.39 (m, 1H), 7.24 (d, 1H), 7.03 (t, 1H), 6.62 (d, 1H), 6.15 (s, 1H), 5.23 (dd, 1H), 3.91 (s, 3H), 3.91 (br. s., 2H), 3.78 (s, 2H), 3.76 (s, 3H), 3.35-3.41 (m, 2H), 2.13-2.17 (m, 3H), 2.13 (s, 3H). MS m/z 483 [M−H]⁻.

### Example 34

### (R)-2-(8-(3-Methoxy-4-(5-methyl-1H-imidazol-1-yl)phenylamino)-2-phenyl-2,3-dihydropyrido[3,2-f][1,4]oxazepin-4(5H)-yl)ethanol

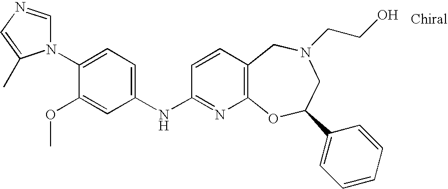

(R)-2-(8-Chloro-2-phenyl-2,3-dihydropyrido[3,2-f][1,4]oxazepin-4(5H)-yl)ethanol (Example 26a, 97 mg, 0.32 mmol), 3-methoxy-4-(5-methyl-1H-imidazol-1-yl)aniline (84 mg, 0.41 mmol), Pd(OAc)₂ (7.15 mg, 0.03 mmol), 2-(dicyclohexylphosphino)biphenyl (11.15 mg, 0.03 mmol) and Cs₂CO₃ (311 mg, 0.95 mmol) were weighed into a microwave vial. The vial was capped and flushed with argon. DME (2 mL) and ethanol (1 mL) were added and the mixture was heated to 100° C. in a microwave apparatus for 1 h. Pd(OAc)₂ (7.15 mg, 0.03 mmol) and 2-(dicyclohexylphosphino)biphenyl (11.15 mg, 0.03 mmol) were freshly added and the heating was repeated. The cooled reaction mixture was diluted with dichloromethane, filtered through a pad of Celite and concentrated. The residue was purified by preparative HPLC and then purified again by column chromatography on Silica using gradient elution with increasing concentration of methanol from 0 to 5% in dichloromethane giving 15 mg of the title compound (10% Yield).

¹H NMR (500 MHz, DMSO-d₆) δ ppm 9.32 (s, 1H) 7.70 (d, 1H) 7.56 (d, 1H) 7.48 (d, 2H) 7.44 (s, 1H) 7.39 (t, 2H) 7.29-7.35 (m, 1H) 7.18 (dd, 1H) 7.09 (d, 1H) 6.70 (s, 1H) 6.63 (d, 1H) 5.13 (d, 1H) 4.47 (t, 1H) 3.84 (q, 2H) 3.66 (s, 3H) 3.55 (q, 2H) 3.15-3.23 (m, 1H) 3.07-3.15 (m, 1H) 2.64 (t, 2H) 1.93 (s, 3H). MS m/z 472 [M+H]⁺ 470 [M−H]⁻.

### Example 34a

### 3-methoxy-4-(5-methyl-1H-imidazol-1-yl)aniline

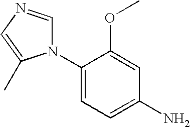

A mixture of 4-methyl imidazole (500 mg, 6.1 mmol), 2-fluoro-5-nitro anisole (1.02 g, 5.9 mmol) and potassium carbonate (1.68 g, 12 mmol) in DMF (15 mL) was heated overnight at 85° C. in a sealed tube. The reaction mixture was cooled, transferred into a round bottom flask using ethyl acetate and concentrated under high vacuum to 5 mL volume. The residue was suspended in water and extracted with dichloromethane. The organic extracts were combined, washed with brine, dried over anhydrous MgSO₄, filtered and concentrated. The residue was dissolved in dichloromethane (10 mL) and diluted with hexane until the solution became slightly turbid. The turbid solution was left at room temperature. The separated solid was filtered, washed with hexane to give 1-(2-methoxy-4-nitro-phenyl)-4-methyl-1H-imidazole. The mother liquor was purified by preparative HPLC giving 0.20 g of 1-(2-methoxy-4-nitro-phenyl)-5-methyl-1H-imidazole.

10% Pd/C (0.28 g, 2.6 mmol) was added to a solution of 1-(2-methoxy-4-nitro-phenyl)-5-methyl-1H-imidazole (0.61 g, 2.6 mmol) in ethyl acetate (20 mL). The mixture was hydrogenated at 35 psi over night. The mixture was filtrated through celite and concentrated to about 10 mL. Diethylether (50 mL) was added and the solution was cooled to 0° C. 4M HCl in dioxane (2 mL) was added and the solution was stirred for 15 min then warmed up to rt and stirred for 30 min. The excess solvent was decanted off and more diethylether was added and the mixture stirred for 15 min. This was repeated once more with a large amount of diethylether. The wet solid was dried under vacuum giving 0.60 g of the title compound (16% Yield).

¹H NMR (400 MHz, METHANOL-d4) ppm 2.15 (s, 3H), 3.92 (s, 3H), 7.07 (dd, 1H), 7.19 (d, 1H), 7.49 (s, 1H), 7.59 (d, 1H), 9.03 (d, 1H)

### Example 35

### 2-(Isopropoxymethyl)-N-(3-methoxy-4-(4-methyl-1H-imidazol-1-yl)phenyl)-4-methyl-2,3,4,5-tetrahydropyrido[3,2-f][1,4]oxazepin-8-amine

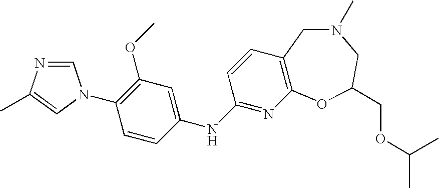

The title compound was prepared in an analogous procedure as described in Example 31 using 8-chloro-2-(isopropoxymethyl)-4-methyl-2,3,4,5-tetrahydropyrido[3,2-f][1,4]oxazepine and 3-methoxy-4-(4-methyl-1H-imidazol-1-yl)aniline as starting material (5% Yield).

¹H NMR (500 MHz, DMSO-d₆) δ ppm 9.24 (s, 1H), 7.67-7.70 (m, 1H), 7.63 (d, 1H), 7.46 (d, 1H), 7.26 (dd, 1H), 7.18 (d, 1H), 7.01 (d, 1H), 6.55 (d, 1H), 4.22 (t, 1H), 4.03-4.12 (m, 1H), 3.79 (s, 3H), 3.57-3.66 (m, 3H), 3.45-3.50 (m, 3H), 2.29 (s, 3H), 2.14 (s, 3H), 1.10 (dd, 6H). MS m/z 438 [M+H]⁺ 436 [M−H]⁻.

### Example 35a

### 8-chloro-2-(isopropoxymethyl)-4-methyl-2,3,4,5-tetrahydropyrido[3,2-f][1,4]oxazepine

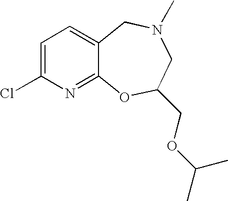

A mixture of formaldehyde (0.033 mL, 1.20 mmol), 8-chloro-2-(isopropoxymethyl)-2,3,4,5-tetrahydropyrido[3,2-f][1,4]oxazepine (0.293 g, 1.14 mmol) and acetic acid (0.033 mL, 0.57 mmol) in methanol (5 mL) was stirred at 0° C. for 15 minutes. Sodium cyanoborohydride (0.072 g, 1.14 mmol) was added. After 5 minutes the ice water bath was removed and the reaction was stirred at room temperature for 1 hour. The reaction mixture was concentrated and partitioned between sat. NaHCO₃ (aq) and dichloromethane. The organic layer was washed with brine and water, dried (MgSO₄) and concentrated giving 0.278 g of the title compound (90% Yield).

¹H NMR (500 MHz, DMSO-d₆) δ ppm 7.72 (d, 1H), 7.19 (d, 1H), 4.14-4.21 (m, 1H), 3.75 (dt, 1H), 3.56-3.63 (m, 2H), 3.49 (dd, 2H), 2.88-2.94 (m, 1H), 2.78-2.84 (m, 1H), 2.30 (s, 3H), 1.11 (d, 3H), 1.10 (d, 3H). MS m/z 271 [M+H]⁺.

### Example 36

### 2-(8-(3-Methoxy-4-(4-methyl-1H-imidazol-1-yl)phenylamino)-2-(pyridin-2-yl)-2,3-dihydropyrido[3,2-f][1,4]oxazepin-4(5H)-yl)acetonitrile

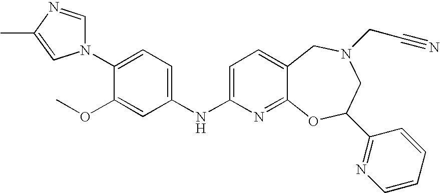

The title compound was prepared in an analogous procedure as described in Example 26 using 2-(8-chloro-2-(pyridin-2-yl)-2,3-dihydropyrido[3,2-f][1,4]oxazepin-4(5H)-yl)acetonitrile and 3-methoxy-4-(4-methyl-1H-imidazol-1-yl)aniline as starting material. The crude product was purified on preparative HPLC yielding 35 mg of the title compound (24% Yield)

¹H NMR (500 MHz, DMSO-d₆) δ ppm 9.36 (s, 1H) 8.54-8.62 (m, 1H) 7.91 (td, 1H) 7.83 (d, 1H) 7.74-7.79 (m, 1H) 7.62 (d, 1H) 7.55 (d, 1H) 7.36-7.43 (m, 1H) 7.17-7.22 (m, 1H) 7.10-7.16 (m, 1H) 6.98-7.02 (m, 1H) 6.64 (d, 1H) 5.10-5.17 (m, 1H) 3.90-4.01 (m, 2H) 3.72-3.87 (m, 5H) 3.50-3.58 (m, 1H) 3.09 (dd, 1H) 2.09-2.16 (m, 3H). MS m/z 468 [M+H]⁺466 [M−H]⁻.

### Example 36a

### 2-(8-chloro-2-(pyridin-2-yl)-2,3-dihydropyrido[3,2-f][1,4]oxazepin-4(5H)-yl)acetonitrile

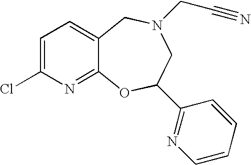

To a solution of 8-chloro-2-(pyridin-2-yl)-2,3,4,5-tetrahydropyrido[3,2-f][1,4]oxazepine (96 mg, 0.37 mmol) in THF (1 mL) was added triethylamine (0.102 mL, 0.73 mmol) and 2-bromoacetonitrile (66.0 mg, 0.55 mmol). The resulting mixture was stirred for 78 h at room temperature. Additional bromoacetonitrile (40 mg) and triethylamine (0.080 mL) was added after one day. The reaction mixture was diluted with ethyl acetate, washed with dilute aqueous HCl (pH ˜4) and with saturated aqueous NaHCO₃. The organics was dried over MgSO₄ and concentrated to give 97 mg of the title product (88% Yield).

¹H NMR (400 MHz, DMSO-d₆) δ ppm 8.59 (m, 1H), 7.93 (td, 1H), 7.81 (d, 1H), 7.70 (d, 1H), 7.42 (ddd, 1H), 7.31 (d, 1H), 5.28 (dd, 1H), 4.05 (m, 1H), 3.97 (d, 2H), 3.83 (d, 1H), 3.46 (m, 1H), 3.20 (dd, 1H). MS m/z 301, 303 [M+H]⁺299, 301 [M−H]⁻.

### Example 36b

### 8-chloro-2-(pyridin-2-yl)-2,3,4,5-tetrahydropyrido[3,2-f][1,4]oxazepine

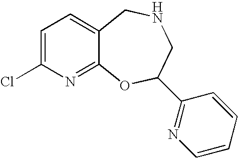

A 60% suspension of NaH (0.160 g, 4.01 mmol) in mineral oil was washed with heptane and suspended in THF (4 mL) under an argon atmosphere. This was cooled in an ice-water bath and a solution of 2-((2,6-dichloropyridin-3-yl)methylamino)-1-(pyridin-2-yl)ethanol (1.138 g, 3.82 mmol) in THF (6 mL) was added drop wise. The mixture was stirred at 0° C. for 15 min and then warmed to room temperature. The mixture was stirred for 16 h at rt. Another portion of NaH (40 mg) was added and stirring was continued for 4 h and then a second addition of NaH (40 mg) was done and the temperature was raised to 50° C. for 3 h. The reaction was quenched by the addition of water. The mixture was diluted with ethyl acetate and washed with water, the organic layer was dried over MgSO₄ and concentrated. The residue was purified by column chromatography on Silica using gradient elution (MeOH-DCM, 0-6%) to give 294 mg of the title product (Yield 29%).

¹H NMR (400 MHz, DMSO-d₆) δ ppm 8.53-8.59 (m, 1H) 7.90 (td, 1H) 7.77 (d, 1H) 7.62-7.66 (m, 1H) 7.37 (ddd, 1H) 7.24 (d, 1H) 5.01 (dd, 1H) 4.02 (d, 1H) 3.79 (d, 1H) 3.45 (dd, 1H) 3.34 (s, 1H) 3.09 (dd, 1H). MS m/z 262, 264 [M+H]⁺260, 262 [M−H]⁻.

### Example 36c

### 2-((2,6-dichloropyridin-3-yl)methylamino)-1-(pyridin-2-yl)ethanol

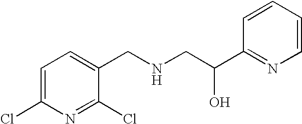

The title compound was prepared in an analogous procedure as described in Example 14c using 2-amino-1-(pyridin-2-yl)ethanol as starting material (53% Yield) ¹H NMR (400 MHz, DMSO-d₆) δ ppm 8.43-8.51 (m, 1H) 7.95 (d, 1H) 7.78 (td, 1H) 7.56 (d, 1H) 7.50 (d, 1H) 7.25 (ddd, 1H) 5.58 (d, 1H) 4.67-4.75 (m, 1H) 3.81 (s, 2H) 2.90 (dd, 1H) 2.70 (dd, 1H). MS m/z 298, 290, 292 [M+H]⁺.

### Example 37

### N-(3-Methoxy-4-(4-methyl-1H-imidazol-1-yl)phenyl)-4-methyl-2-(pyridin-2-yl)-2,3,4,5-tetrahydropyrido[3,2-f][1,4]oxazepin-8-amine

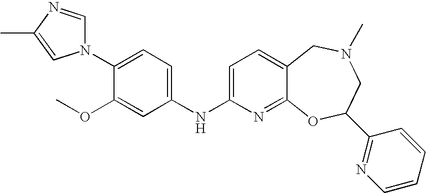

A solution of 8-chloro-4-methyl-2-(pyridin-2-yl)-2,3,4,5-tetrahydropyrido[3,2-f][1,4]oxazepine (155 mg, 0.56 mmol) in DME (4 mL) was added to a microwave vial containing 3-methoxy-4-(4-methyl-1H-imidazol-1-yl)aniline (149 mg, 0.73 mmol), Pd(OAc)₂ (12.62 mg, 0.06 mmol), 2-(dicyclohexylphosphino)biphenyl (19.70 mg, 0.06 mmol) and Cs₂CO₃ (549 mg, 1.69 mmol). The vial was immediately evacuated and flushed with argon. The resulting mixture was heated to 100° C. in a microwave for 1 h. Fresh catalyst and ligand were added and the heating was repeated. The cooled reaction mixture was diluted with DCM and filtered through a pad of Celite. The filtrate was concentrated to dryness and the residue was first purified by HPLC and then by column chromatography on silica to give 12 mg of the title product (5% Yield).

¹H NMR (500 MHz, DMSO-d₆) δ ppm 9.31 (s, 1H) 8.52-8.58 (m, 1H) 7.89 (td, 1H) 7.82 (d, 1H) 7.75 (d, 1H) 7.62 (d, 1H) 7.56 (d, 1H) 7.33-7.38 (m, 1H) 7.16-7.20 (m, 1H) 7.10-7.15 (m, 1H) 6.97-7.02 (m, 1H) 6.63 (d, 1H) 5.08 (d, 1H) 3.64-3.79 (m, 5H) 3.34-3.40 (m, 1H) 2.97 (dd, 1H) 2.38 (s, 3H) 2.12 (s, 3H). MS m/z 443 [M+H]⁺441 [M−H]⁻.

### Example 37a

### 8-chloro-4-methyl-2-(pyridin-2-yl)-2,3,4,5-tetrahydropyrido[3,2-f][1,4]oxazepine

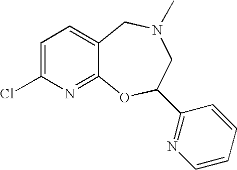

Formaldehyde (642 mg, 7.91 mmol) was added to a solution of 8-chloro-2-(pyridin-2-yl)-2,3,4,5-tetrahydropyrido[3,2-f][1,4]oxazepine (207 mg, 0.79 mmol) in acetonitrile (1 mL). The resulting mixture was stirred at room temperature for 1 h and then was NaCNBH₄ (149 mg, 2.37 mmol) and acetic acid (0.453 mL, 7.91 mmol) added. The mixture was stirred over night, then the solvent was evaporated and the residue was partitioned between dichloromethane and saturated aqueous NaHCO₃. The phases were separated and the organic phase was dried over MgSO₄ and concentrated. The residue was purified by column chromatography on Silica using a gradient of methanol in dichloromethane (0-5%) to give 169 mg of a mixture comprising the expected product together with some unidentified side-product. The material was used in the following step without further purification.

Mixture of rotamers ¹H NMR (400 MHz, DMSO-d₆) δ ppm 8.63 (m) 8.05 (d) 7.92-8.01 (m) 7.75 (d) 7.38-7.49 (m) 5.78-5.90 (m) 4.49 (d) 4.31-4.38 (m) 4.22-4.31 (m) 3.57-3.87 (m, 2H) 2.85 (s) 2.74 (s)

Total number of protons in spectrum: 14

Ratio major:minor: 1.1:1

MS m/z 276, 278 [M+H]⁺.

### Example 38

### (R)-2-(3-(3-methoxy-4-(4-methyl-1H-imidazol-1-yl)phenylamino)-6-phenyl-6,7-dihydropyrazino[2,3-f][1,4]oxazepin-8(9H)-yl)acetonitrile

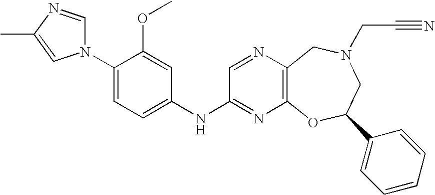

Pd₂(dba)₃ (16 mg, 5 mol %) and rac-BINAP (22 mg, 10 mol %) were added to a solution of (R)-2-(3-chloro-6-phenyl-6,7-dihydropyrazino[2,3-f][1,4]oxazepin-8(9H)-yl)acetonitrile (107 mg, 0.36 mmol) and 3-methoxy-4-(4-methyl-imidazol-1-yl)-phenylamine (94 mg, 0.46 mmol) in anhydrous toluene (6 mL) at room temperature The mixture was degassed for 10 minutes and NaOBu^{t} (55 mg, 0.57 mmol) was added. The reaction mixture was degassed for an additional 5 minutes and then heated at 90° C. for 18 hours. The reaction mixture was cooled to room temperature, diluted with CH₂Cl₂, filtered and washed with H₂O. The organic layer was dried over MgSO₄, filtered and concentrated under reduced pressure. The residue was first chromatographed using 4 to 6% MeOH in CH₂Cl₂: EtOAc:mixture (1:1) to afford a crude product which was further purified by preparatory HPLC (MeCN gradient in 0.1% aqueous NH₄OH). The fractions were combined and concentrated until precipitation occurred. The material was collected by filtration, washed with water and dried to yield 71 mg of the title compound (43% Yield)

¹H NMR (400 MHz, CD₃OD) δ ppm 7.87 (s, 1H), 7.72 (d, 1H), 7.65 (bs, 1H), 7.54-7.49 (m, 2H), 7.44-7.33 (m, 3H), 7.25 (dd, 1H), 7.17 (d, 1H), 6.95 (bs, 1H), 5.25 (dd, 1H), 4.12 (d, 1H), 4.02 (d, 1H), 3.76 (s, 3H), 3.29-3.24 (m, 2H), 2.93 (s, 3H). MS m/z: 468 [M+1]⁺.

### Example 38a

### (R)-2-(3-chloro-6-phenyl-6,7-dihydropyrazino[2,3-f][1,4]oxazepin-8(9H)-yl)acetonitrile

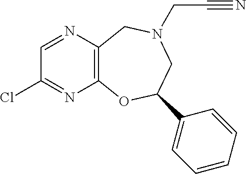

Potassium carbonate (271 mg, 1.96 mmol) and bromoacetonitrile (118 mg, 0.98 mmol) were added to a stirred solution of (R)-3-chloro-6-phenyl-6,7,8,9-tetrahydropyrazino[2,3-f][1,4]oxazepine (Example 39b, 257 mg, 0.98 mmol) in anhydrous acetonitrile (8 mL) under N₂ at room temperature. The reaction mixture was stirred for 20 h and then partitioned between H₂O (50 mL) and EtOAc (75 mL). The organic layer was separated, washed with brine, dried over MgSO₄, filtered and concentrated. The residue was purified by flash column chromatography using 40% EtOAc in hexanes to afford 230 mg of the title compound (78% Yield).

¹H NMR (400 MHz, CDCl₃) δ ppm 8.28 (s, 1H), 7.49-7.33 (m, 5H), 5.21 (dd, 1H), 4.23 (dd, 1H), 4.08 (d, 1H), 3.77 (m, 2H), 3.37 (dd, 1H), 3.21 (dt, 1H)

### Example 39

### (R)—N-(3-Methoxy-4-(4-methyl-1H-imidazol-1-yl)phenyl)-8-methyl-6-phenyl-6,7,8,9-tetrahydropyrazino[2,3-f][1,4]oxazepin-3-amine

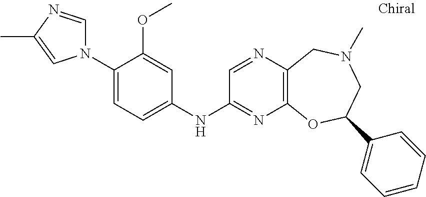

Pd₂(dba)₃ (12 mg, 5 mol %) and rac-BINAP (17 mg, 10 mol %) were added to a solution of (R)-3-chloro-8-methyl-6-phenyl-6,7,8,9-tetrahydropyrazino[2,3-f][1,4]oxazepine (124 mg, 0.45 mmol) and 3-methoxy-4-(4-methyl-1H-imidazol-1-yl)aniline (119 mg, 0.59 mmol) in anhydrous toluene (6 mL) at room temperature. The resulting mixture was degassed for 10 minutes, NaOBu^{t} (70 mg, 0.73 mmol) was added and the solution was degassed for an additional 5 minutes. The reaction mixture was heated at 80° C. for 4 hours, then cooled to room temperature, diluted with CH₂Cl₂ and filtered. The organic phase was washed with H₂O, dried over MgSO₄, filtered and concentrated under reduced pressure. Purification by flash column chromatography using a gradient of 5 to 9% MeOH in CH₂Cl₂, followed by purification via preparatory HPLC (MeCN gradient in 0.1% aqueous NH₄OH) afforded 115 mg of the title compound (54% Yield).

¹H NMR (400 MHz, DMSO-d₆) δ 7.90 (s, 1H), 7.74 (d, 1H), 7.65 (d, 1H), 7.53-7.49 (m, 2H), 7.43-7.37 (m, 2H), 7.37-7.32 (m, 1H), 7.26 (dd, 1H), 6.95 (s, 1H), 7.20 (d, 1H), 5.20 (dd, 1H), 4.01 (d, 1H), 3.93 (d, 1H), 3.77 (s, 3H), 3.18-3.11 (m, 2H), 2.53 (s, 3H), 2.21 (s, 3H). MS m/z: 441 [M−1]⁻.

### Example 39a

### (R)-3-chloro-8-methyl-6-phenyl-6,7,8,9-tetrahydropyrazino[2,3-f][1,4]oxazepine

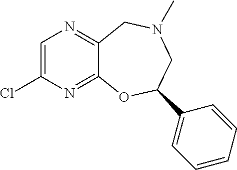

A mixture of (R)-3-chloro-6-phenyl-6,7,8,9-tetrahydropyrazino[2,3-f][1,4]oxazepine (0.25 g, 0.96 mmol and paraformaldehyde (1.00 g, 33.3 mmol) in methanol (20 mL) was stirred at room temperature for 1 h. Sodium triacetoxyborohydride (1.00 g, 4.70 mmol) was added and the reaction mixture was stirred for 24 h at room temperature. The reaction mixture was then concentrated and the residue was chromatographed (SiO₂, 3% MeOH in CH₂Cl₂) to give 220 mg of the title compound (85% Yield)

¹H NMR (CDCl₃): δ 8.28 (s, 1H), 7.45-7.31 (m, 5H), 5.18 (dd, 1H), 4.13 (dd, 1H), 3.97 (d, 1H), 3.22 (dd, 1H), 3.12 (dt, 1H), 2.54 (s, 3H). MS m/z: 276.12 [M+1]⁺.

### Example 39b

### (R)-3-chloro-6-phenyl-6,7,8,9-tetrahydropyrazino[2,3-f][1,4]oxazepine

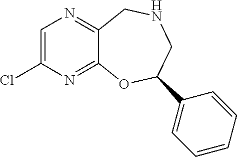

Sodium hydride (79 mg, 3.16 mmol, 95% dry) was added to a stirred solution of (R)-2-((3,5-dichloropyrazin-2-yl)methylamino)-1-phenylethanol (856 mg, 2.07 mmol) in anhydrous THF (40 mL) under N₂ at −30° C. The mixture was warmed to room temperature and stirred for 20 h. The reaction mixture was diluted with EtOAc and water. The organic phase was separated, washed with H₂O, brine, dried over MgSO₄, filtered and concentrated under reduced pressure. The crude product was purified by flash column chromatography using 3 to 4% MeOH in CH₂Cl₂/EtOAc mixture (1:1) to give 520 mg of the title compound (69% Yield).

¹H NMR (400 MHz, CDCl₃) δ ppm 8.25 (s, 1H), 7.45-7.31 (m, 5H), 5.10 (dd, 1H), 4.37 (d, 1H), 4.17 (d, 1H), 3.44 (dd, 1H), 3.33 (dd, 1H)

### Example 39c

### (R)-2-((3,5-dichloropyrazin-2-yl)methylamino)-1-phenylethanol

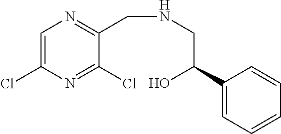

A mixture of 3,5-dichloropyrazine-2-carbaldehyde (828 mg, 4.68 mmol, prepared as described in J. Organomet. Chem., 1991, 412(3) 301-310) and (R)-2-amino-1-phenyl-ethanol (769 mg, 5.61 mmol) in 1,2-dichloroethane (20 mL) was stirred at 0° C. for 1 hour before the addition of sodium triacetoxyborohydride (1.189 g, 5.61 mmol). The reaction mixture was allowed to warm to room temperature and stirred for an additional 18 hours. The mixture was diluted with CH₂Cl₂ and washed with water. The organic layer was dried over MgSO₄, filtered and concentrated under reduced pressure. The residue was purified by flash column chromatography using 4 to 5% MeOH in CH₂Cl₂/EtOAc mixture (1:1) to afford 795 mg of the title compound (57% Yield).

¹H NMR (400 MHz, CDCl₃): δ ppm 8.48 (s, 1H), 7.40-7.28 (m, 5H), 4.79 (dd, 1H), 4.12 (m, 2H), 2.97 (dd, 1H), 2.94-2.60 (m, 3H). MS m/z: 298 [M+1]⁺.

### Example 40

### (R)-8-(3-Methoxy-4-(4-methyl-1H-imidazol-1-yl)phenylamino)-N,N-dimethyl-2-phenyl-2,3-dihydropyrido[3,2-f][1,4]oxazepine-4(5H)-carboxamide

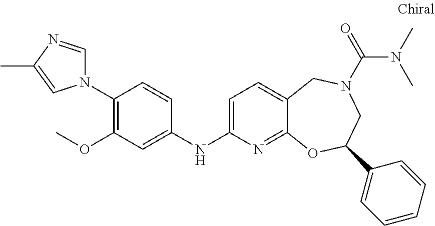

(R)-8-chloro-N,N-dimethyl-2-phenyl-2,3-dihydropyrido[3,2-f][1,4]oxazepine-4(5H)-carboxamide (92 mg, 0.28 mmol), 3-methoxy-4-(4-methyl-1H-imidazol-1-yl)aniline (73.3 mg, 0.36 mmol), Pd(OAc)₂ (6.23 mg, 0.03 mmol), 2-(dicyclohexylphosphino)biphenyl (9.72 mg, 0.03 mmol) and Cs₂CO₃ (271 mg, 0.83 mmol) were weighed into a microwave vial. The vial was capped and flushed with argon. DME (4 mL) was added and the mixture was heated to 100° C. for 2 h. Fresh catalyst and ligand was added and the reaction was continued at 100° C. for another 2 h. The reaction mixture was diluted with DCM, filtered and evaporated. The residue was purified by preparative HPLC then further purified by column chromatography on Silica using gradient elution with increasing concentration of methanol, from 0 to 5%, in DCM to give 59 mg of the title product (43% Yield).

¹H NMR (500 MHz, DMSO-d₆) δ ppm 9.28 (s, 1H) 7.70 (s, 1H) 7.61 (d, 1H) 7.57 (d, 1H) 7.53-7.55 (m, 1H) 7.52 (br. s., 1H) 7.41-7.45 (m, 2H) 7.34-7.39 (m, 1H) 7.16-7.19 (m, 2H) 6.99 (t, 1H) 6.58 (d, 1H) 5.41 (dd, 1H) 4.55 (d, 1H) 4.24 (d, 1H) 3.79 (dd, 1H) 3.72 (s, 3H) 3.50 (dd, 1H) 2.73 (s, 6H) 2.12 (d, 3H). MS m/z 499 [M+H]⁺497 [M−H]⁻.

### Example 40a

### (R)-8-chloro-N,N-dimethyl-2-phenyl-2,3-dihydropyrido[3,2-f][1,4]oxazepine-4(5H)-carboxamide

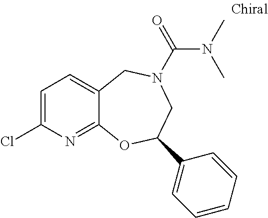

N-Ethyldiisopropylamine (0.119 mL, 0.68 mmol) and dimethylcarbamic chloride (0.058 mL, 0.63 mmol) were sequentially added to a solution of (R)-8-chloro-2-phenyl-2,3,4,5-tetrahydropyrido[3,2-f][1,4]oxazepine (161 mg, 0.62 mmol) in DCM (3 mL). The mixture was stirred at room temperature for 16 h. The reaction mixture was diluted with DCM and washed with 1 M aqueous citric acid, water and saturated aqueous NaHCO₃. The solution was dried over Na₂SO₄ and concentrated to give 194 mg of the title compound (95% Yield).

¹H NMR (400 MHz, DMSO-d6) δ ppm 7.84 (d, 1H) 7.48-7.53 (m, 2H) 7.36-7.47 (m, 3H) 7.21 (d, 1H) 5.61 (dd, 1H) 4.69 (d, 1H) 4.36 (d, 1H) 3.76 (dd, 1H) 3.59 (dd, 1H) 2.70 (s, 6H). MS m/z 332, 334 [M+H]⁺330, 332 [M−H]⁻.

### Example 41

### (R)-2-(8-(3-Methoxy-4-(3-methyl-1H-1,2,4-triazol-1-yl)phenylamino)-2-phenyl-2,3-dihydropyrido[3,2-f][1,4]oxazepin-4(5H)-yl)ethanol

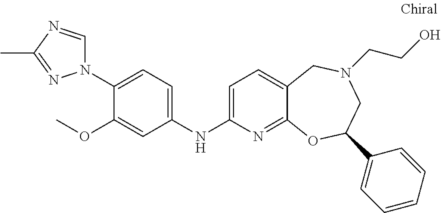

3-Methoxy-4-(3-methyl-1H-1,2,4-triazol-1-yl)aniline (67.0 mg, 0.33 mmol), (R)-2-(8-chloro-2-phenyl-2,3-dihydropyrido[3,2-f][1,4]oxazepin-4(5H)-yl)ethanol (100 mg, 0.33 mmol), palladium acetate (7.37 mg, 0.03 mmol), 2-(dicyclohexylphosphino)biphenyl (11.50 mg, 0.03 mmol) and cesium carbonate (321 mg, 0.98 mmol) were added to a microwave vial. 1,2-Dimethoxyethane (2 mL) was added. The reaction mixture was flushed with argon and the mixture was run in a microwave for 60 minutes at 100° C. Additional catalyst and ligand were added and the reaction was run 1 h at 100° C. EtOH (0.500 mL) was added and the reaction mixture was run 1 h at 100° C. The solids were filtered off and the solvent was concentrated. The crude product was purified twice with column chromatography on Silica with 20-80% MeOH in DCM as eluent giving 33 mg of the title compound (21% Yield).

¹H NMR (400 MHz, DMSO-d₆) δppm 2.31 (s, 3H) 2.64 (t, 2H) 3.08-3.25 (m, 2H) 3.55 (q, 2H) 3.74 (s, 3H) 3.78-3.91 (m, 2H) 4.48 (t, 1H) 5.14 (d, 1H) 6.63 (d, 1H) 7.14 (dd, 1H) 7.29-7.36 (m, 1H) 7.36-7.43 (m, 3H) 7.46-7.52 (m, 2H) 7.57 (d, 1H) 7.83 (d, 1H) 8.60 (s, 1H) 9.34 (s, 1H). MS m/z 473 [M+H]⁺ 471 [M−H]⁻.

### Example 41a

### 3-methoxy-4-(3-methyl-1H-1,2,4-triazol-1-yl)aniline

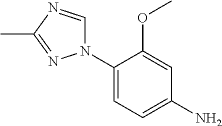

Pd on carbon (10%, wet) was added to a solution of 1-(2-methoxy-4-nitrophenyl)-3-methyl-1H-1,2,4-triazole (2.5 g, 10.67 mmol) in ethyl acetate (25 mL) and methanol (25 mL). The resulting mixture was shaken under hydrogen atmosphere (30 psi) overnight at room temperature. The mixture was filtered through a pad of Celite and the solids were washed with ethyl acetate-methanol (2×25 mL). The filtrate was concentrated under reduced pressure to afford 2.0 g of the title compound (92% Yield).

¹H NMR (400 MHz, CHLOROFORM-d) δ ppm 2.47 (s, 3H) 3.82 (s, 3H) 3.84 (br. s., 2H) 6.28-6.38 (m, 2H) 7.37 (d, 1H) 8.35 (s, 1H). MS m/z: 205 [M+1]⁺.

### Example 41b

### 1-(2-methoxy-4-nitrophenyl)-3-methyl-1H-1,2,4-triazole

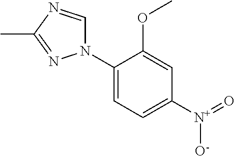

A mixture of 3-methyl-1H-[1,2,4]triazole (2.4 g, 29.21 mmol), 1-fluoro-2-methoxy-4-nitrobenzene (5.0 g, 29.21 mmol) and potassium carbonate (8.06 g, 58.42 mmol) in DMF (50 mL) was heated overnight at 85° C. in a pressure vessel. The reaction mixture was cooled to room temperature and concentrated under reduced pressure. The residue was suspended in water and the mixture extracted with dichloromethane. The organic extracts were combined, washed with brine, dried over anhydrous Na₂SO₄, filtered and concentrated. The residue was dissolved in dichloromethane (50 mL) and hexane was added in small portions until the solution become slightly turbid. The turbid solution was left at room temperature. The precipitation was collected by filtration, washed with hexane to give 2.5 g of title compound (37% Yield).

¹H NMR (400 MHz, CHLOROFORM-d) δ ppm 2.51 (s, 3H) 4.10 (s, 3H) 7.90-8.06 (m, 2H) 8.10 (d, 1H) 8.87 (s, 1H)

### Example 42

### (R)-2-(8-(4-(4-Methyl-1H-imidazol-1-yl)phenylamino)-2-phenyl-2,3-dihydropyrido[3,2-f][1,4]oxazepin-4(5H)-yl)ethanol

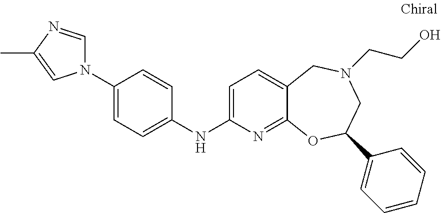

The title compound was prepared in an analogous procedure as described in Example 26 using a mixture of 4-(4-methyl-1H-imidazol-1-yl)aniline (76 mg, 0.44 mmol) and 4-(5-methyl-1H-imidazol-1-yl)aniline together with (R)-2-(8-chloro-2-phenyl-2,3-dihydropyrido[3,2-f][1,4]oxazepin-4(5H)-yl)ethanol (Example 26a) as starting material. A Supercritical fluid chromatography (SFC) was performed in order to separate the regioisomers giving 17 mg of the title compound (7% Yield).

¹H NMR (500 MHz, DMSO-d₆) δ ppm 9.16 (s, 1H) 7.95 (d, 1H) 7.66 (d, 2H) 7.52 (d, 1H) 7.45-7.50 (m, 2H) 7.39-7.45 (m, 4H) 7.31-7.36 (m, 1H) 7.29 (s, 1H) 6.57 (d, 1H) 5.10 (dd, 1H) 4.45 (t, 1H) 3.75-3.89 (m, 2H) 3.53 (q, 2H) 3.07-3.14 (m, 2H) 2.62 (t, 2H) 2.12 (s, 3H). MS m/z 442.2 [M+H]⁺.

### Example 43

### (R)-2-(8-(5-methoxy-6-(4-methyl-1H-imidazol-1-yl)pyridin-3-ylamino)-2-phenyl-2,3-dihydropyrido[3,2-f][1,4]oxazepin-4(5H)-yl)ethanol

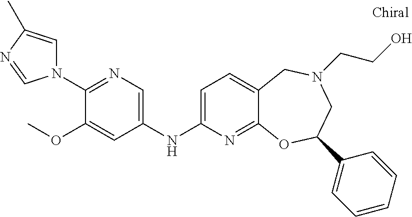

(R)-2-(8-chloro-2-phenyl-2,3-dihydropyrido[3,2-f][1,4]oxazepin-4(5H)-yl)ethanol (Example 26a, 0.252 g, 0.83 mmol), 5-methoxy-6-(4-methyl-1H-imidazol-1-yl)pyridin-3-amine (0.169 g, 0.83 mmol) (isomeric mixture), palladium (II) acetate (0.019 g, 0.08 mmol), 2-(dicyclohexylphosphino)biphenyl (0.029 g, 0.08 mmol) and cesium carbonate (0.808 g, 2.48 mmol) were placed in a 5 mL microwave vial and sealed and purged with argon. The reaction mixture was run in the microwave at 100° C. for 60 minutes. Added 0.080 g (R)-2-(8-chloro-2-phenyl-2,3-dihydropyrido[3,2-f][1,4]oxazepin-4(5H)-yl)ethanol and 0.1 eq of palladium (II) acetate and 2-(dicyclohexylphosphino)biphenyl and the reaction mixture was run in the microwave again for 45 minutes at 100° C. The reaction mixture was filtered through celite and concentrated. The crude product was purified by column chromatography on Silica using a gradient of methanol (0 to 7%) in dichloromethane giving 0.023 g of the title compound (6% Yield).

¹H NMR (500 MHz, DMSO-d₆) δ ppm 9.48 (s, 1H) 8.26-8.32 (m, 1H) 8.14-8.19 (m, 1H) 8.01-8.06 (m, 1H) 7.56-7.62 (m, 1H) 7.46-7.53 (m, 2H) 7.38-7.43 (m, 2H) 7.37 (t, 1H) 7.31-7.35 (m, 1H) 6.62 (d, 1H) 5.15 (ddd, 1H) 4.47 (t, 1H) 3.83-3.90 (m, 2H) 3.79-3.83 (m, 3H) 3.55 (q, 2H) 3.18-3.24 (m, 1H) 3.14 (d, 1H) 2.62-2.66 (m, 2H) 2.11-2.15 (m, 3H). MS m/z 473 [M+H]⁺ 471 [M−H]⁻.

### Example 43a

### 5-methoxy-6-(4-methyl-1H-imidazol-1-yl)pyridin-3-amine

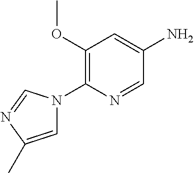

To a stirred solution of 3-methoxy-2-(4-methyl-1H-imidazol-1-yl)-5-nitropyridine (0.913 g, 3.90 mmol) (isomeric mixture) in methanol:dichloromethane (1:1, 60 mL) was added nickel (II) chloride (0.126 g, 0.97 mmol) followed by sodium borohydride (0.442 g, 11.69 mmol) in small portions. The resulting mixture was stirred at room temperature for 20 minutes. 5 g silica gel was added and the reaction mixture was concentrated and filtered. The crude product was purified by column chromatography on Silica using a gradient of methanol (0 to 5%) in DCM giving 0.655 g of the title compound as an isomeric mixture (82% Yield).

¹H NMR (400 MHz, DMSO-d₆) δ ppm 7.38 (d, 1H), 7.21 (s, 1H), 6.81 (d, 1H), 5.55 (s, 2H), 3.79 (s, 3H), 2.12 (d, 4H). MS m/z 205 [M+H]⁺ 203 [M−H]⁻.

### Example 43b

### 3-methoxy-2-(4-methyl-1H-imidazol-1-yl)-5-nitropyridine

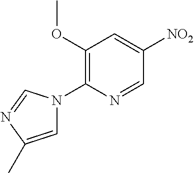

To a suspension of 4-methyl-1H-imidazole (0.522 g, 6.36 mmol) and potassium carbonate (1.832 g, 13.26 mmol) in DMSO (20 mL) at room temperature was added 2-chloro-3-methoxy-5-nitropyridine (1.0 g, 5.30 mmol). The reaction mixture was stirred at 45° C. overnight. About 20 mL warm water (heated to 50° C.) was added to the reaction mixture and the precipitated solid was immediately filtrated and washed with warm water. The solid was dried in the dry vacuum oven at 40° C. for two days giving 0.914 g of the title compound as an isomeric mixture (74% Yield).

¹H NMR (400 MHz, DMSO-d₆) δ ppm 8.90-8.94 (m, 1H), 8.45 (s, 1H), 8.32-8.37 (m, 1H), 7.66-7.70 (m, 1H), 4.10 (s, 3H), 2.15-2.20 (m, 3H). MS m/z 235 [M+H]⁺.

### Example 44

### (R)—N-(3-Methoxy-4-(3-methyl-1H-1,2,4-triazol-1-yl)phenyl)-4-methyl-2-phenyl-2,3,4,5-tetrahydropyrido[3,2-f][1,4]oxazepin-8-amine

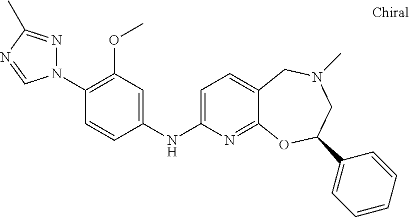

(R)-8-Chloro-4-methyl-2-phenyl-2,3,4,5-tetrahydropyrido[3,2-f][1,4]oxazepine (Example 9a, 180 mg, 0.66 mmol), 3-methoxy-4-(3-methyl-1H-1,2,4-triazol-1-yl)aniline (134 mg, 0.66 mmol), palladium acetate (14.71 mg, 0.07 mmol), 2-(dicyclohexylphosphino)biphenyl (22.96 mg, 0.07 mmol) and cesium carbonate (640 mg, 1.97 mmol) were mixed in DME (3 mL) in a microwave vial. EtOH (0.3 mL) was added and the mixture was run for 1 h at 100° C. in a microwave reactor. The reaction had to be run for an additional 2 hours with more catalyst and ligand added. The mixture was filtered and purified with column chromatography on Silica using 0-10% MeOH in DCM as eluent giving 55 mg of the title compound (19% Yield).

¹H NMR (500 MHz, CHLOROFORM-d) δ ppm 8.49 (s, 1H) 7.58 (d, 1H) 7.45 (t, 3H) 7.38 (t, 2H) 7.31-7.35 (m, 2H) 6.86 (dd, 1H) 6.77 (br. s., 1H) 6.68 (d, 1H) 5.13 (d, 1H) 3.87 (d, 1H) 3.83 (s, 3H) 3.68 (d, 1H) 3.07-3.20 (m, 2H) 2.51 (s, 3H) 2.48 (s, 3H).

MS m/z 443 [M+H]⁺ 441 [M−H]⁻.

### Example 45

### (R)-4-Cyclopropyl-N-(3-methoxy-4-(3-methyl-1H-1,2,4-triazol-1-yl)phenyl)-2-phenyl-2,3,4,5-tetrahydropyrido[3,2-f][1,4]oxazepin-8-amine

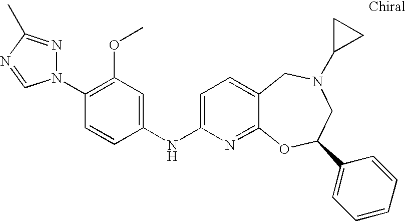

The title compound was prepared in an analogous procedure as described in Example 44 using (R)-8-chloro-4-cyclopropyl-2-phenyl-2,3,4,5-tetrahydropyrido[3,2-f][1,4]oxazepine (180 mg, 0.60 mmol) and 3-methoxy-4-(3-methyl-1H-1,2,4-triazol-1-yl)aniline as starting material (6% Yield).

¹H NMR (500 MHz, CHLOROFORM-d) δ ppm 8.50 (s, 1H) 7.59 (d, 1H) 7.44-7.52 (m, 3H) 7.39 (t, 2H) 7.33 (t, 1H) 7.29 (br. s., 1H) 6.84 (dd, 1H) 6.67 (d, 1H) 6.64 (br. s., 1H) 5.15 (d, 1H) 3.96-4.09 (m, 1H) 3.88 (d, 1H) 3.84 (s, 3H) 3.34 (d, 1H) 3.25 (br. s., 1H) 2.49 (s, 3H) 2.06 (d, 1H) 0.56 (br. s., 4H). MS m/z 469 [M+H]⁺467 [M−H]⁻.

### Example 45a

### (R)-8-chloro-4-cyclopropyl-2-phenyl-2,3,4,5-tetrahydropyrido[3,2-f][1,4]oxazepine

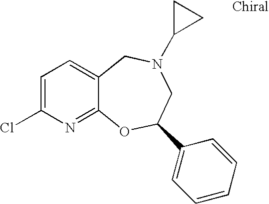

(R)-8-chloro-2-phenyl-2,3,4,5-tetrahydropyrido[3,2-f][1,4]oxazepine (Example 11b, 200 mg, 0.77 mmol), (1-ethoxycyclopropoxy)trimethylsilane (0.617 mL, 3.07 mmol), acetic acid (0.438 mL, 7.67 mmol) and 3 Å molecular sieves were stirred in MeOH (15 mL) at room temp. Sodium cyanoborohydride (96 mg, 1.53 mmol) was carefully added and the mixture was then heated to reflux over 8 hours. The reaction mixture was filtered trough celite and concentrated giving 180 mg of the title compound (78% Yield)

MS m/z 301 [M+H].

### Example 46

### 2-(8-(3-Methoxy-4-(4-methyl-1H-imidazol-1-yl)phenylamino)-2-phenyl-2,3-dihydropyrido[3,2-f][1,4]oxazepin-4(5H)-yl)ethanol

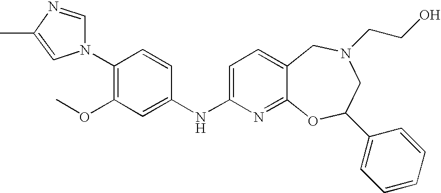

2-(8-Chloro-2-phenyl-2,3-dihydropyrido[3,2-f][1,4]oxazepin-4(5H)-yl)ethanol (86 mg, 0.28 mmol), 3-methoxy-4-(4-methyl-1H-imidazol-1-yl)aniline (74.6 mg, 0.37 mmol), Pd(OAc)₂ (6.34 mg, 0.03 mmol), 2-(dicyclohexylphosphino)biphenyl (9.89 mg, 0.03 mmol) and Cs₂CO₃ (276 mg, 0.85 mmol) were weighed into a microwave vial. The vial was capped and flushed with argon. DME (3 mL) was added and the mixture was heated to 100° C. for 2 h. Fresh catalyst and ligand was added and the reaction was continued at 100° C. for another 2 h. The reaction mixture was diluted with DCM, filtered and evaporated. The residue was purified by preparative HPLC and then further purified by column chromatography on Silica using gradient elution with increasing concentration of methanol, from 0 to 5%, in DCM to give 42 mg of the title product (32% Yield).

¹H NMR (500 MHz, DMSO-d₆) δ ppm 9.26 (s, 1H) 7.67 (s, 1H) 7.60 (s, 1H) 7.54 (d, 1H) 7.48 (d, 2H) 7.39 (t, 2H) 7.29-7.35 (m, 1H) 7.14-7.20 (m, 2H) 6.99 (s, 1H) 6.60 (d, 1H) 5.12 (d, 1H) 4.46 (t, 1H) 3.83-3.89 (m, 1H) 3.77-3.83 (m, 1H) 3.70 (s, 3H) 3.54 (q, 2H) 3.15-3.22 (m, 1H) 3.07-3.15 (m, 1H) 2.63 (t, 2H) 2.11 (s, 3H). MS m/z 472 [M+H]⁺470 [M−H]⁻.

### Example 46a

### 2-(8-chloro-2-phenyl-2,3-dihydropyrido[3,2-f][1,4]oxazepin-4(5H)-yl)ethanol

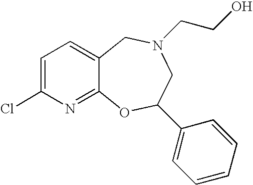

2-Hydroxyacetaldehyde (46.1 mg, 0.77 mmol) was added to a solution of 8-chloro-2-phenyl-2,3,4,5-tetrahydropyrido[3,2-f][1,4]oxazepine (Example 14b, 160 mg, 0.61 mmol) in methanol (2 mL). The resulting solution was allowed to react at room temperature for 45 min. Acetic acid (0.035 mL, 0.61 mmol) and NaCNBH₄ (77 mg, 1.23 mmol) were sequentially added and the reaction mixture was stirred for 1 h. Additional hydroxyacetaldehyde (25.00 mg, 0.42 mmol) was added and stirring was continued for 10 min before addition of NaCNBH₄ (48.0 mg, 0.76 mmol). The mixture was stirred for 5 h at room temperature. The reaction was quenched by addition of saturated aqueous NaHCO₃. The aqueous mixture was extracted with dichloromethane. The combined extracts were dried over Na₂SO₄ and evaporated. The residue was purified by column chromatography on Silica using gradient elution with increasing concentration of methanol in DCM to give 95 mg of the title product (51% Yield).

¹H NMR (400 MHz, DMSO-d6) δ ppm 7.82 (d, 1H) 7.31-7.50 (m, 5H) 7.25 (d, 1H) 5.22 (dd, 1H) 4.45-4.53 (m, 1H) 3.88-4.03 (m, 2H) 3.49-3.57 (m, 2H) 3.11-3.23 (m, 2H) 2.63 (td, 2H). MS m/z 305, 307 [M+H]⁺303, 305 [M−H]⁻.

### Example 47

### (R)-2-(8-(3-Methoxy-4-(4-methyl-1H-imidazol-1-yl)phenylamino)-2-phenyl-2,3-dihydropyrido[3,2-f][1,4]oxazepin-4(5H)-yl)acetonitrile

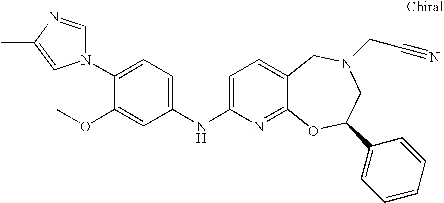

2-Bromoacetonitrile (0.087 mL, 1.25 mmol) was added to a solution of (R)-8-chloro-2-phenyl-2,3,4,5-tetrahydropyrido[3,2-f][1,4]oxazepine (Example 14b, 250 mg, 0.96 mmol) and TEA (0.174 mL, 1.25 mmol) in DCM (5 mL) and the reaction mixture is stirred at room temperature. The solvent was evaporated and the residue was partitioned between sat. NaHCO₃ (aq) and dichloromethane. The organic layer was washed with water, dried (MgSO₄) and concentrated. The residue, 3-Methoxy-4-(4-methyl-1H-imidazol-1-yl)aniline (195 mg, 0.96 mmol), palladium acetate (21.55 mg, 0.10 mmol) and cesium carbonate (938 mg, 2.88 mmol) were added in a microwave vial. The mixture was capped and flushed with argon. 1,2-Dimethoxyethane (5 mL) was added, the reaction mixture was flushed with argon and the mixture was run in a microwave for 60 minutes at 100° C. After two additional additions of catalyst and ligand with 3 h extra runtime was the reaction complete. The reaction mixture was filtrated through Celite and washed with dichloromethane. The filtrate was concentrated and the crude product was purified by to preparative chromatography (3 runs) yielding 35.0 mg of the title compound (8% Yield).

¹H NMR (500 MHz, CHLOROFORM-d) δ ppm 2.33 (s, 3H) 3.17 (d, 1H) 3.31 (dd, 1H) 3.64-3.75 (m, 2H) 3.75-3.81 (m, 4H) 4.00 (d, 1H) 5.11 (d, 1H) 6.69 (d, 1H) 6.75 (s, 1H) 6.83-6.90 (m, 2H) 7.16 (d, 1H) 7.26 (d, 1H) 7.33-7.43 (m, 3H) 7.47 (dd, 3H) 7.74 (s, 1H). MS m/z 467 [M+H]⁺ 465 [M−H]⁻.

### Example 48

### (R)-4-Methyl-N-(4-(3-methyl-1H-1,2,4-triazol-1-yl)phenyl)-2-phenyl-2,3,4,5-tetrahydropyrido[3,2-f][1,4]oxazepin-8-amine

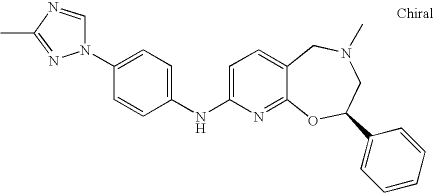

DME was added to a mixture of (R)-8-chloro-4-methyl-2-phenyl-2,3,4,5-tetrahydropyrido[3,2-f][1,4]oxazepine (250 mg, 0.91 mmol), 4-(3-methyl-1H-1,2,4-triazol-1-yl)aniline (159 mg, 0.91 mmol), acetoxy(2′-(di-tert-butylphosphino)biphenyl-2-yl)palladium (21.06 mg, 0.05 mmol) and cesium carbonate (445 mg, 1.36 mmol) in a microwave vial which was then immediately flushed with argon. The resulting mixture was heated to 100° C. in a microwave apparatus for 1 h. The mixture was diluted with dichloromethane, filtered and concentrated. The residue was purified by column chromatography on Silica using gradient elution with increasing concentration of methanol, from 0 to 8%, in dichloromethane to give 138 mg of the title product (37% Yield).

¹H NMR (500 MHz, DMSO-d₆) δ ppm 9.24 (s, 1H), 8.94 (s, 1H), 7.70 (m, 2H), 7.63 (m, 2H), 7.54 (d, 1H), 7.49 (m, 2H), 7.41 (t, 2H), 7.34 (m, 1H), 6.60 (d, 1H), 5.09 (t, 1H), 3.68 (q, 2H), 2.97 (m, 2H), 2.35 (s, 3H), 2.32 (s, 3H). MS m/z 413 [M+H]⁺411 [M−H]⁻.

### Example 48a

### 4-(3-methyl-1H-1,2,4-triazol-1-yl)aniline

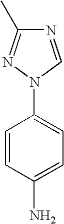

Palladium on activated carbon (50%, wet, 2.5 g) was added to a solution of 3-methyl-1-(4-nitrophenyl)-1H-1,2,4-triazole (950 mg, 4.6 mmol) in THF (20 mL) and the resulting mixture was stirred under atmospheric pressure of hydrogen at room temperature overnight. The reaction mixture was filtered through a pad of Celite and concentrated under reduced pressure. The crude product was purified by flash column chromatography using 50-90% ethyl acetate in DCM to afford 700 mg of the title compound (87% Yield)

¹H NMR (400 MHz, CHLOROFORM-d) δ ppm 2.48 (s, 3H) 3.86 (br.s, 2H) 6.72-6.76 (m, 2H) 7.35-7.39 (m, 2H) 8.26 (s, 1H). MS m/z: 175 [M+1]⁺.

### Example 48b

### 3-methyl-1-(4-nitrophenyl)-1H-1,2,4-triazole

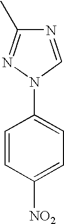

3-methyl-1H-1,2,4-triazole (648 mg, 7.8 mmol) and potassium carbonate (2.0 g, 14.2 mmol) were added to a solution of 1-fluoro-4-nitro-benzene (1.0 g, 7.1 mmol) in DMF (10 mL). The reaction mixture was heated in a sealed tube at 80° C. overnight, cooled to room temperature and filtered through a pad of Celite. The solids were washed with dichloromethane and the filtrate was concentrated under reduced pressure. The residue was taken in water and the resultant precipitate was collected by filtration, washed with water and dried under high vacuum to afford 950 mg of the title compound (67% Yield).

¹H NMR (400 MHz, CHLOROFORM-d) δ ppm 2.51 (s, 3H) 7.83-7.89 (m, 2H) 8.36-8.41 (m, 2H) 8.58 (s, 1H). MS m/z: 205 [M+1]⁺.

### Example 49

### 2-Cyclopropyl-N-(3-methoxy-4-(4-methyl-1H-imidazol-1-yl)phenyl)-4-methyl-2,3,4,5-tetrahydropyrido[3,2-f][1,4]oxazepin-8-amine

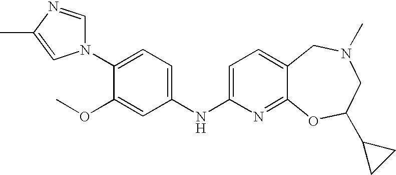

The crude product from Example 49a, Example 49b and Example 49c were combined and purified by column chromatography on Silica using a gradient of methanol (0 to 5%) in dichloromethane. The product was purified again by column chromatography on Silica using a eluent of 7N ammonia in methanol (10%) in DCM in dichloromethane giving 0.141 g of the title compound (19% Yield).

¹H NMR (500 MHz, DMSO-d₆) δ ppm 9.24 (s, 1H), 7.94 (d, 1H), 7.64 (d, 1H), 7.45 (d, 1H), 7.19 (d, 1H), 7.08 (dd, 1H), 7.02 (s, 1H), 6.53 (d, 1H), 3.82 (s, 3H), 3.51 (s, 2H), 3.40 (t, 1H), 2.99 (d, 1H), 2.80 (dd, 1H), 2.28 (s, 3H), 2.14 (s, 3H), 0.99-1.09 (m, 1H), 0.48-0.57 (m, 2H), 0.35-0.47 (m, 2H). MS m/z 406 [M+H]⁺ 404 [M−H]⁻.

### Example 49a

### 2-cyclopropyl-N-(3-methoxy-4-(4-methyl-1H-imidazol-1-yl)phenyl)-4-methyl-2,3,4,5-tetrahydropyrido[3,2-f][1,4]oxazepin-8-amine

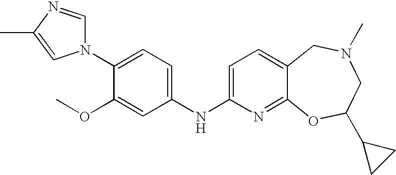

3-methoxy-4-(4-methyl-1H-imidazol-1-yl)aniline (0.189 g, 0.93 mmol), 8-chloro-2-cyclopropyl-4-methyl-2,3,4,5-tetrahydropyrido[3,2-f][1,4]oxazepine (0.222 g, 0.93 mmol), palladium acetate (0.021 g, 0.09 mmol), 2-(dicyclohexylphosphino)biphenyl (0.033 g, 0.09 mmol) and cesium carbonate (0.303 g, 0.93 mmol) were added in a microwave vial. The mixture was capped and flushed with argon. 1,2-dimethoxyethane (2 mL) was added and the mixture was run in a microwave for 60 minutes at 100° C. Palladium acetate (0.1 eq) and 2-(dicyclohexylphosphino)biphenyl (0.1 eq) and cesium carbonate (1 eq) were added and the reaction run for another 60 min at 100° C. in the microwave. A small spatula of acetoxy(2′-(di-tert-butylphosphino)biphenyl-2-yl)palladium and ethanol (0.3 mL) were added and the reaction run for 30 minutes at 100° C. The reaction mixture was filtrated through celite and concentrated. The crude product was purified by column chromatography on Silica using a gradient of methanol (+10% 7M ammonia) in dichloromethane. Obtained 0.203 mg of impure product which was combined with Example 49b and Example 49c and purified in Example 49.

### Example 49b

### 2-cyclopropyl-N-(3-methoxy-4-(4-methyl-1H-imidazol-1-yl)phenyl)-4-methyl-2,3,4,5-tetrahydropyrido[3,2-f][1,4]oxazepin-8-amine

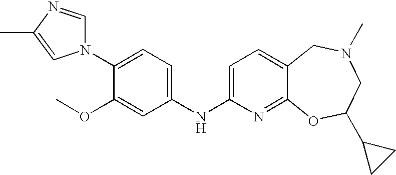

A reaction mixture of 8-chloro-2-cyclopropyl-4-methyl-2,3,4,5-tetrahydropyrido[3,2-f][1,4]oxazepine (0.111 g, 0.46 mmol), 3-methoxy-4-(4-methyl-1H-imidazol-1-yl)aniline (0.095 g, 0.46 mmol), acetoxy(2′-(di-tert-butylphosphino)biphenyl-2-yl)palladium (10.76 mg, 0.02 mmol) and cesium carbonate (0.227 g, 0.70 mmol) in DME (2 mL) was heated in the microwave at 100° C. for 45 minutes. Added 0.05 eq of acetoxy(2′-(di-tert-butylphosphino)biphenyl-2-yl)palladium and run again in the microwave at 100° C. for 60 minutes. The reaction mixture was filtered through celite and concentrated. The crude was combined with Example 49a and Example 49c and purified in Example 49.

### Example 49c

### 2-cyclopropyl-N-(3-methoxy-4-(4-methyl-1H-imidazol-1-yl)phenyl)-4-methyl-2,3,4,5-tetrahydropyrido[3,2-f][1,4]oxazepin-8-amine

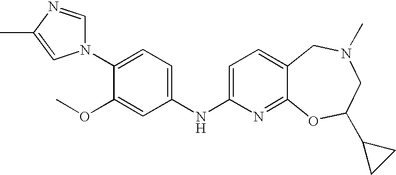

A reaction mixture of 8-chloro-2-cyclopropyl-4-methyl-2,3,4,5-tetrahydropyrido[3,2-f][1,4]oxazepine (0.111 g, 0.46 mmol), 3-methoxy-4-(4-methyl-1H-imidazol-1-yl)aniline (0.095 g, 0.46 mmol), acetoxy(2′-(di-tert-butylphosphino)biphenyl-2-yl)palladium (10.76 mg, 0.02 mmol) and cesium carbonate (0.303 g, 0.93 mmol) in DME (2 mL) was heated in the microwave at 100° C. for 60 minutes. The reaction mixture was filtered through celite and concentrated. The crude was combined with Example 49a and Example 49b and purified in Example 49.

### Example 49d

### 8-chloro-2-cyclopropyl-4-methyl-2,3,4,5-tetrahydropyrido[3,2-f][1,4]oxazepine

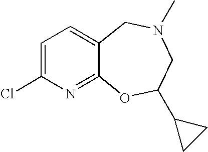

Formaldehyde (0.040 mL, 1.44 mmol) was added to a stirred solution of 8-chloro-2-cyclopropyl-2,3,4,5-tetrahydropyrido[3,2-f][1,4]oxazepine (0.323 g, 1.44 mmol) in anhydrous methanol (6 mL) at room temperature. A few drops of acetic acid were added followed by cyanoborohydride (Polymer-supported) (0.654 g, 1.51 mmol). The reaction mixture was stirred at room temperature overnight. The reaction mixture was filtrated and the filtrated was concentrated giving 0.298 g of the title compound (87% Yield) which was used in the next step without further purification.

¹H NMR (500 MHz, DMSO-d₆) δ ppm 7.70 (d, 1H), 7.16 (d, 1H), 3.70 (d, 1H), 3.57 (d, 1H), 3.43-3.50 (m, 1H), 2.97 (d, 1H), 2.86-2.93 (m, 1H), 2.29 (s, 3H), 0.98-1.12 (m, 1H), 0.48-0.63 (m, 2H), 0.33-0.47 (m, 2H). MS m/z 239 [M+H]⁺.

### Example 49e

### 8-chloro-2-cyclopropyl-2,3,4,5-tetrahydropyrido[3,2-f][1,4]oxazepine

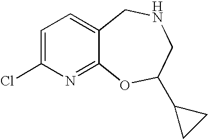

Sodium hydride (0.034 g, 1.43 mmol) was added to a stirred solution of 1-cyclopropyl-2-((2,6-dichloropyridin-3-yl)methylamino)ethanol (0.312 g, 1.19 mmol) in THF (8 mL) at 0° C. The icewater bath was removed after 30 minutes. After 3 hr, NaH (0.5 eq., dispersed in mineral oil) was added and the reaction continued overnight. NaH (0.5 eq., dispersed in mineral oil) was added and after 5 h, methanol was added drop wise until it stopped bubbling. The solvent was evaporated and the residue was partitioned between sat. NaHCO₃ (aq) and dichloromethane. The organic layer was separated and the water layer was extracted twice with dichloromethane. The combined organic layer was dried (NaSO₄) and concentrated giving the title compound in quantitative yield.

MS m/z 225, 227 [M+H]⁺.

### Example 49f

### 1-cyclopropyl-2-((2,6-dichloropyridin-3-yl)methylamino)ethanol

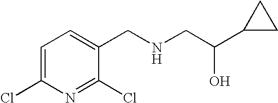

To a stirred solution of 2,6-dichloronicotinaldehyde (0.609 g, 3.46 mmol) and 2-amino-1-cyclopropylethanol (0.350 g, 3.46 mmol) in 1,2-dichloroethane (20 mL) was added two drops of acetic acid followed by sodium triacetoxyborohydride (2.20 g, 10.38 mmol) at room temperature. The reaction mixture was stirred at room temperature overnight. Sat. NaHCO₃ (aq) was added in portions to the reaction mixture. DCM was added and the organic layer was separated. The water layer was extracted twice with DCM. The combined organic layers was dried (Na₂SO₄) and concentrated. The crude product was purified by silica column chromatography using a gradient of methanol (0 to 5%) in dichloromethane giving 0.314 g of the title compound (35% Yield).

¹H NMR (500 MHz, DMSO-d₆) δ ppm 8.02 (d, 1H), 7.58 (d, 1H), 4.57 (d, 1H), 3.77 (s, 2H), 2.90-3.03 (m, 1H), 2.56-2.62 (m, 1H), 2.51-2.55 (m, 1H), 2.28 (br. s., 1H), 0.80 (qt, 1H), 0.28-0.41 (m, 2H), 0.10-0.28 (m, 2H). MS m/z 262.9 [M+H]⁺.

### Example 49g

### 2-amino-1-cyclopropylethanol

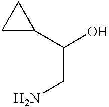

A mixture of 1-cyclopropyl-2-nitro-ethanol (7.6 g, 57.88 mmol) and wet palladium on activated carbon (10%, 0.76 g, 10% w/w) in methanol (100 mL) was shaken under a hydrogen atmosphere (32 psi) for 24 hours. The reaction mixture was filtered through a pad of Celite and concentrated under reduced pressure to afford 5.87 g of the title compound (100% Yield).

¹H NMR (400 MHz, CHLOROFORM-d) δ ppm 0.17-0.27 (m, 1H) 0.31-0.41 (m, 1H) 0.45-0.59 (m, 2H) 0.80-0.92 (m, 1H) 2.58 (br. s., 3H) 2.73-2.82 (m, 1H) 2.87-2.95 (m, 1H) 2.96-3.05 (m, 1H). MS m/z: 101 [M+1]⁺.

### Example 49h

### 1-Cyclopropyl-2-nitro-ethanol

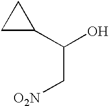

NaOH (576 mg, 14.41 mmol) dissolved in water (10 mL) was added to a stirred mixture of cyclopropanecarbaldehyde (1.0 g, 14.27 mmol) and nitromethane (0.87 g, 14.27 mmol) in methanol (15.0 mL) at 0° C. The reaction mixture was allowed to warm slowly over 1 h to room temperature and was quenched by addition of acetic acid (1.5 mL). The volatiles were removed in vacuo and the residue was redissolved in water. The aqueous phase was neutralized using saturated solution of NaHCO₃ and extracted with ethyl acetate. The combined organic extracts were dried over Na₂SO₄, filtered and concentrated under reduced pressure to afford 1.6 g of the title compound (86% Yield).

¹H NMR (400 MHz, CHLOROFORM-d) δ ppm 0.31-0.40 (m, 1H) 0.43-0.51 (m, 1H) 0.59-0.72 (m, 2H) 0.88-1.02 (m, 1H) 2.40 (br. s., 1H) 3.68 (td, 1H) 4.54-4.62 (m, 2H)

### Example 50

### (R)—N-(6-Methoxy-5-(1-methyl-1H-pyrazol-4-yl)pyridin-2-yl)-4-methyl-2-phenyl-2,3,4,5-tetrahydropyrido[3,2-f][1,4]oxazepin-8-amine

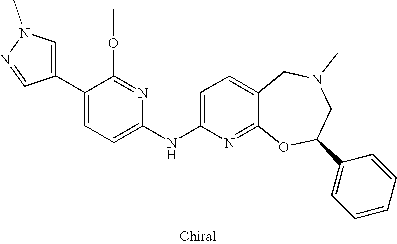

6-Methoxy-5-(1-methyl-1H-pyrazol-4-yl)pyridin-2-amine (123 mg, 0.60 mmol), (R)-8-chloro-4-methyl-2-phenyl-2,3,4,5-tetrahydropyrido[3,2-f][1,4]oxazepine (Example 9a, 150 mg, 0.55 mmol), cesium carbonate (320 mg, 0.98 mmol) and acetoxy(2′-(di-tert-butylphosphino)biphenyl-2-yl)palladium (12.64 mg, 0.03 mmol) were added to a microwave vial. 1,2-Dimethoxyethane (1 mL) and EtOH (0.100 mL) were added. The reaction mixture was flushed with argon and the mixture was run in the microwave oven to for 30 minutes at 100° C. The solids were filtered off and washed with DCM. The solvent was evaporated and the crude product was purified twice using straight phase chromatography, 0-10% MeOH in DCM, yielding 70.0 mg of the title compound (29.0% Yield)

¹H NMR (500 MHz, DMSO-d₆) δ ppm 9.65 (s, 1H) 8.01 (s, 1H) 7.81-7.87 (m, 2H) 7.71 (d, 1H) 7.63 (d, 1H) 7.49 (d, 2H) 7.41 (t, 2H) 7.35 (d, 1H) 6.96 (d, 1H) 5.10 (dd, 1H) 4.01 (s, 3H) 3.86 (s, 3H) 3.67-3.80 (m, 2H) 2.98-3.04 (m, 2H) 2.37 (s, 3H). MS m/z 443.2 [M+H]⁺.

### Example 50b

### 6-methoxy-5-(1-methyl-1H-pyrazol-4-yl)pyridin-2-amine

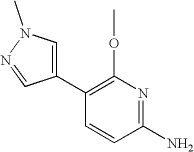

1-Methyl-4-(4,4,5,5-tetramethyl-1,3,2-dioxaborolan-2-yl)-1H-pyrazole (205 mg, 0.99 mmol), potassium carbonate (340 mg, 2.46 mmol) and Pd 118 (32.1 mg, 0.05 mmol) were added to 5-bromo-6-methoxypyridin-2-amine (200 mg, 0.99 mmol) in dioxane (3 mL). Water (0.5 mL) was added and the reaction was heated to 110° C. for 45 min in the microwave oven. The solids were filtered off and washed with DCM. The solvent was evaporated and the crude product was purified on silica column chromatography using a gradient of 0-10% MeOH in DCM yielding 118 mg of the title compound (59% Yield).

¹H NMR (500 MHz, DMSO-d₆) δ ppm 7.87 (s, 1H) 7.71 (s, 1H) 7.59 (d, 1H) 6.06 (d, 1H) 5.84 (s, 2H) 3.86 (s, 3H) 3.83 (s, 3H). MS m/z 205 [M+H]⁺.

### Example 51

### 2-Cyclopropyl-N-(3-methoxy-4-(3-methyl-1H-1,2,4-triazol-1-yl)phenyl)-4-methyl-2,3,4,5-tetrahydropyrido[3,2-f][1,4]oxazepin-8-amine

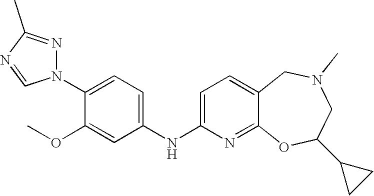

The title compound was prepared in an analogous procedure as described in Example 48 using 8-chloro-2-cyclopropyl-4-methyl-2,3,4,5-tetrahydropyrido[3,2-f][1,4]oxazepine (Example 49d) and 3-methoxy-4-(3-methyl-1H-1,2,4-triazol-1-yl)aniline (Example 41a) as starting material (33.0% Yield).

¹H NMR (500 MHz, DMSO-d₆) δ ppm 9.32 (s, 1H), 8.64 (s, 1H), 8.04 (d, 1H), 7.46 (d, 1H), 7.41 (d, 1H), 7.09 (dd, 1H), 6.55 (d, 1H), 3.87 (s, 3H), 3.52 (s, 2H), 3.40 (t, 1H), 3.00 (d, 1H), 2.80 (dd, 1H), 2.32 (s, 3H), 2.28 (s, 3H), 1.00-1.09 (m, 1H), 0.49-0.57 (m, 2H), 0.35-0.49 (m, 2H). MS m/z 407 [M+H]⁺ 405 [M−H]⁻.

### Example 52

### N-(3-Methoxy-4-(3-methyl-1H-1,2,4-triazol-1-yl)phenyl)-4-methyl-2-(6-methylpyridin-2-yl)-2,3,4,5-tetrahydropyrido[3,2-f][1,4]oxazepin-8-amine

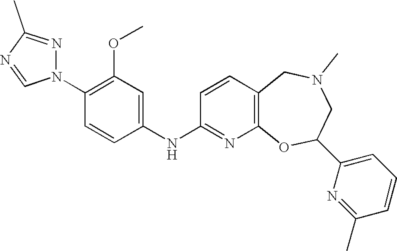

3-Methoxy-4-(3-methyl-1H-1,2,4-triazol-1-yl)aniline (Example 41a, 6.4 mg, 0.28 mmol), cesium carbonate (162 mg, 0.50 mmol) and acetoxy(2′-(di-tert-butylphosphino)biphenyl-2-yl)palladium (6.39 mg, 0.01 mmol) were added to a microwave vial. 8-Chloro-4-methyl-2-(6-methylpyridin-2-yl)-2,3,4,5-tetrahydropyrido[3,2-f][1,4]oxazepine 0.69 M in DME (0.400 mL, 0.28 mmol) was added followed by DME (2 mL). The reaction was set under N₂ atmosphere and heated to 110° C. for 1.25 hour. More catalyst (6 mg) was added and heated again in the microwave to 110° C. for 1 h.

The solids were filtered off and washed with DCM. The solvent was evaporated and the crude product was purified using straight phase chromatography, 0-10% MeOH in DCM. Then purified again by preparative HPLC using 15 to 50% gradient, acidic method (0.2% formic acid/methanol) giving 33 mg of the title compound (26% Yield).

¹H NMR (400 MHz, CDCl₃) δ ppm 8.49 (s, 1H) 7.55-7.65 (m, 2H) 7.50-7.54 (m, 1H) 7.43 (d, 1H) 7.32 (d, 1H) 7.07 (d, 1H) 6.85 (dd, 1H) 6.80 (s, 1H) 6.65 (d, 1H) 5.13-5.21 (m, 1H) 3.91 (d, 1H) 3.84 (s, 3H) 3.67 (d, 1H) 3.43 (d, 1H) 3.08 (dd, 1H) 2.54 (s, 3H) 2.50 (s, 3H) 2.48 (s, 3H). MS m/z 457 [M+1]⁺.

### Example 52a

### 8-chloro-4-methyl-2-(6-methylpyridin-2-yl)-2,3,4,5-tetrahydropyrido[3,2-f][1,4]oxazepine

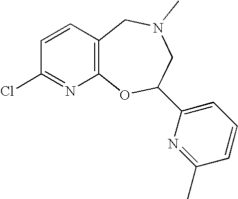

To a solution of 8-chloro-2-(6-methylpyridin-2-yl)-2,3,4,5-tetrahydropyrido[3,2-f][1,4]oxazepine (1.72 g, 6.24 mmol) in methanol (15 mL) was added formaldehyde 37% (4.64 mL, 62.38 mmol) and acetic acid (0.179 mL, 3.12 mmol) at rt under N₂ atmosphere. The mixture was stirred for 15 minutes then sodium cyanoborohydride (0.588 g, 9.36 mmol) was added and allowed to stir for 4 hours at rt. The mixture was treated with NH₃ in methanol to neutralize the acetic acid then the solvent was removed in vacuo. The crude was taken up in ethyl acetate and washed with sat NaHCO₃ solution. The organic layer was dried over anhydrous sodium sulfate, filtered and concentrated. The crude was purified with column chromatography on Silica using heptane:ethyl acetate 100:0 to 0:100 as gradient, then the eluent system was changed to DCM: [DCM:MeOH:NH₃=90:10:1]=100:0 to 30:70 gradient giving 0.813 g of the title compound (45% Yield).

¹H NMR (500 MHz, CDCl₃) δ ppm 7.60-7.65 (m, 1H) 7.51-7.56 (m, 2H) 7.06-7.11 (m, 2H) 5.17 (dd, 1H) 3.94 (d, 1H) 3.76 (dd, 1H) 3.41-3.47 (m, 1H) 3.12 (dd, 1H) 2.53-2.56 (m, 3H) 2.49-2.52 (m, 3H). MS m/z 290 [M+1]⁺.

### Example 52b

### 8-chloro-2-(6-methylpyridin-2-yl)-2,3,4,5-tetrahydropyrido[3,2-f][1,4]oxazepine

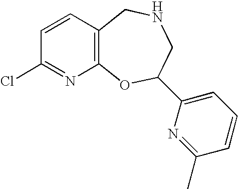

Sodium tert-butoxide (1.053 g, 10.95 mmol) was added in 2 portions to a stirred solution of 2-((2,6-dichloropyridin-3-yl)methylamino)-1-(6-methylpyridin-2-yl)ethanol (2.28 g, 7.30 mmol) in THF (26 mL) at 0° C. The mixture was set under N₂ atmosphere, stirred for 5 minutes at 0° C. and then allowed to warm-up to rt and stirred for 2 days. 2M HCl was added to reach neutral pH (approx 2 mL). The solvent was removed in vacuo. The crude was taken up in methanol and filtered through celite and then concentrated. This procedure was repeated with DCM as well. This gave 1.72 g of the title compound (85% Yield) which was used in the next reaction step without further purification.

¹H NMR (500 MHz, CDCl₃) δ ppm 7.63-7.67 (m, 1H) 7.54 (dd, 2H) 7.06-7.12 (m, 2H) 5.11 (dd, 1H) 4.05-4.17 (m, 2H) 3.78 (dd, 1H) 3.31-3.38 (m, 1H) 2.53 (s, 3H).

MS m/z 276 [M+1]⁺.

### Example 52c

### 2-((2,6-dichloropyridin-3-yl)methylamino)-1-(6-methylpyridin-2-yl)ethanol

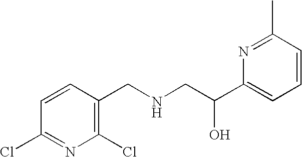

2,6-Dichloronicotinaldehyde (2.079 g, 11.81 mmol), 2-amino-1-(6-methylpyridin-2-yl)ethanol (1.68 g, 11.04 mmol) and acetic acid (0.632 mL, 11.04 mmol) in methanol (21 mL) were stirred at 0° C. for 75 minutes under N₂ atmosphere. Sodium cyanoborohydride (1.041 g, 16.56 mmol) was added in portions and the mixture was stirred at room temperature for 3 h. Thereafter triethylamine (1.539 mL, 11.04 mmol) was added and the solvent was removed in vacuo. The crude product was purified by silica column chromatography using DCM: [DCM:MeOH:NH₃=90:10:1]=100:0 to 40:60 as gradient giving 2.28 g of the title compound (66% Yield).

¹H NMR (400 MHz, CDCl₃) δ ppm 7.92 (d, 1H) 7.63 (t, 1H) 7.31 (d, 1H) 7.19 (d, 1H) 7.12 (d, 1H) 4.97 (dd, 1H) 4.04-4.15 (m, 2H) 3.24 (dd, 1H) 3.02 (dd, 1H) 2.52-2.56 (m, 3H). MS m/z 312, 314 [M+1]⁺.

### Example 52d

### 2-amino-1-(6-methylpyridin-2-yl)ethanol

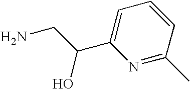

is 6-Methylpicolinaldehyde (3.5 g, 28.89 mmol) in dry DCM (70 mL) was set under N₂ atmosphere and cooled down to 0° C. To this mixture was added drop wise the DCM (5 mL) solution of trimethylsilyl cyanide (4.62 mL, 34.67 mmol) and zinc iodide (9.22 mg, 0.03 mmol). After the addition was complete the mixture was allowed to warm up to rt during 1.5 hours and then the mixture was concentrated. The crude was diluted with ether (70.0 mL) cooled down to 0° C. and lithium aluminum hydride (1.426 g, 37.56 mmol) was added in two portions. The mixture was allowed to warm up to rt overnight then cooled back to 0° C. again and was treated with water (1.44 mL), 15% NaOH solution (1.44 mL), and water (4.32 mL). The resulted precipitates were filtered through celite and washed with methanol (3×). The filtrate was concentrated and the crude was purified by column chromatography using DCM: [MeOH (1% NH₃)]=100:0 to 50:50 gradient giving 1.68 g of the title compound (38% Yield).

¹H NMR (500 MHz, CDCl₃) δppm 7.60 (t, 1H) 7.03-7.15 (m, 2H) 4.70 (dd, 1H) 3.14 (dd, 1H) 2.88 (dd, 1H) 2.56 (s, 3H). MS m/z 153 [M+1]⁺.

### Example 53

### N-(6-Methoxy-5-(1-methyl-1H-pyrazol-4-yl)pyridin-2-yl)-4-methyl-2-(6-methylpyridin-2-yl)-2,3,4,5-tetrahydropyrido[3,2-f][1,4]oxazepin-8-amine

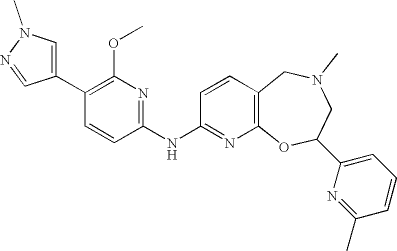

6-Methoxy-5-(1-methyl-1H-pyrazol-4-yl)pyridin-2-amine (Example 50b, 148 mg, 0.72 mmol), cesium carbonate (425 mg, 1.30 mmol) and acetoxy(2′-(di-tert-butylphosphino)biphenyl-2-yl)palladium (16.77 mg, 0.04 mmol) were added to a microwave vial. 8-Chloro-4-methyl-2-(6-methylpyridin-2-yl)-2,3,4,5-tetrahydropyrido[3,2-f][1,4]oxazepine 0.69M in DME (Example 52a, 1.050 mL, 0.72 mmol) was added followed by DME (3 mL) and ethanol (0.15 mL). The reaction was set under N₂ atmosphere and heated in a microwave at 110° C. for 1 h. The solids were filtered off and washed with DCM. The solvent was evaporated and the crude product was purified using column chromatography on Silica using DCM: [DCM:MeOH:NH₃=90:10:1]=100:0 to 20:80 as gradient. The resulted solid was re-purified by preparative HPLC. The title compound was dried in vacuum giving 52 mg (16% Yield).

¹H NMR (400 MHz, CDCl₃) δ ppm 7.77-7.81 (m, 2H) 7.75 (s, 1H) 7.65 (d, 1H) 7.59-7.63 (m, 1H) 7.51 (d, 2H) 7.26 (s, 1H) 7.07 (d, 1H) 6.56 (d, 1H) 5.18 (dd, 1H) 4.06 (s, 3H) 3.91-3.99 (m, 4H) 3.70 (d, 1H) 3.38 (d, 1H) 3.11 (dd, 1H) 2.54 (s, 3H) 2.50 (s, 3H). MS m/z 458 [M+1]⁺.

### Example 54

### 4-Methyl-N-(6-(1-methyl-1H-pyrazol-4-yl)pyridin-3-yl)-2-(6-methylpyridin-2-yl)-2,3,4,5-tetrahydropyrido[3,2-f][1,4]oxazepin-8-amine

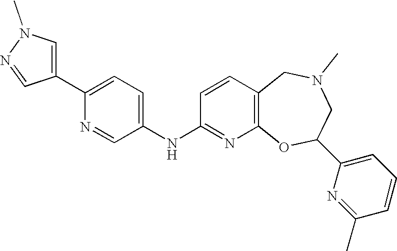

The title compound was prepared in an analogous procedure as described in Example 53 using 6-(1-methyl-1H-pyrazol-4-yl)pyridin-3-amine and 8-chloro-4-methyl-2-(6-methylpyridin-2-yl)-2,3,4,5-tetrahydropyrido[3,2-f][1,4]oxazepine (Example 52b) as starting material (39% Yield, 110 mg).

¹H NMR (500 MHz, CDCl₃) δ ppm 8.48 (d, 1H) 7.88 (d, 2H) 7.71-7.76 (m, 1H) 7.65 (s, 1H) 7.51-7.55 (m, 1H) 7.42 (d, 2H) 7.07-7.12 (m, 1H) 6.59 (d, 1H) 6.41-6.47 (m, 1H) 5.15-5.23 (m, 1H) 3.96 (s, 3H) 3.90-3.95 (m, 1H) 3.64-3.72 (m, 1H) 3.36-3.44 (m, 1H) 3.07-3.15 (m, 1H) 2.56 (s, 3H) 2.51 (s, 3H). MS m/z 428 [M+1]⁺.

### Example 54a

### 6-(1-methyl-1H-pyrazol-4-yl)pyridin-3-amine

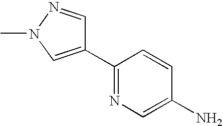

[1,1′-Bis(di-tert-butylphosphino)-ferrocene]palladium (II) dichloride (427.5 mg, 0.66 mmol) was added to a degassed mixture of 5-amino-2-bromopyridine (2.27 g, 13.1 mmol), 1-methyl-4-(4,4,5,5-tetramethyl-1,3,2-dioxaborolan-2-yl)-1H-pyrazole (4.09 g, 19.7 mmol) and potassium carbonate (2.72 g, 19.7 mmol) in dimethoxyethane (80 mL) and water (10 mL). The reaction mixture was heated in a sealed tube at 90° C. overnight, then cooled to room temperature and partitioned between ethyl acetate (100 mL) and aqueous sodium bicarbonate solution (50 mL). The organic phase was separated and the aqueous layer was re-extracted with ethyl acetate. The combined extracts were dried over magnesium sulfate, filtered and concentrated under reduced pressure. The residue was purified by flash column chromatography using a gradient of 2 to 7% methanol in dichloromethane to afford the 1.2 g of the title compound (52% Yield).

¹H NMR (400 MHz, CHLOROFORM-d) δ ppm 3.64 (br. s., 2H) 3.93 (s, 3H) 6.99 (dd, 1H) 7.27 (m, 1H) 7.79 (s, 1H) 7.83 (s, 1H) 8.07 (d, 1H). ESMS m/z: 175 [M+1]⁺.

### Example 55

### (R)—N-(3-Methoxy-4-(4-methyl-1H-imidazol-1-yl)phenyl)-2-phenyl-2,3,4,5-tetrahydropyrido[3,2-f][1,4]oxazepin-8-amine

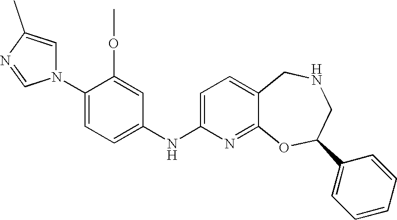

TFA (42.4 μl, 0.55 mmol) was added to a solution of (R)-tert-butyl 8-(3-methoxy-4-(4-methyl-1H-imidazol-1-yl)phenylamino)-2-phenyl-2,3-dihydropyrido[3,2-f][1,4]oxazepine-4(5H)-carboxylate (290 mg, 0.55 mmol) in DCM (3 mL). The reaction mixture was heated to reflux and stirred over night. After cooling the reaction to room temperature, the mixture was neutralised using triethylamine. The solvent was evaporated. The crude product was purified using flash chromatography, 0-10% MeOH in DCM yielding the title compound (110 mg, 46.8%).

¹H NMR (500 MHz, DMSO-d₆) δ ppm 9.61 (s, 1H) 7.90 (s, 1H) 7.76 (d, 1H) 7.71 (d, 1 H) 7.53-7.58 (m, 2H) 7.44-7.50 (m, 2H) 7.39-7.44 (m, 1H) 7.19-7.26 (m, 2H) 7.11 (s, 1H) 6.71 (d, 1H) 5.35 (dd, 1H) 4.22-4.48 (m, 2H) 3.72 (s, 3H) 3.61-3.71 (m, 2H) 2.16 (s, 3H). ESMS m/z: 428.2 [M+1]⁺.

### Example 55a

### (R)-tert-butyl 8-(3-methoxy-4-(4-methyl-1H-imidazol-1-yl)phenylamino)-2-phenyl-2,3-dihydropyrido[3,2-f][1,4]oxazepine-4(5H)-carboxylate

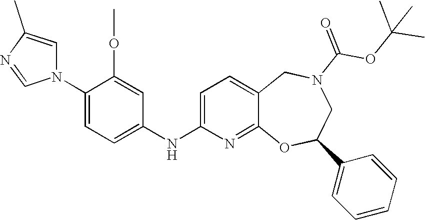

(R)-tert-Butyl 8-chloro-2-phenyl-2,3-dihydropyrido[3,2-f][1,4]oxazepine-4(5H)-carboxylate (200 mg, 0.55 mmol), 3-methoxy-4-(4-methyl-1H-imidazol-1-yl)aniline (113 mg, 0.55 mmol), Cesium carbonate (325 mg, 1.00 mmol) and acetoxy(2′-(di-tert-butylphosphino)biphenyl-2-yl)palladium (12.83 mg, 0.03 mmol) were added to a microwave vial. 1,2-dimethoxyethane (2.5 mL) and EtOH (0.250 mL) were added. The reaction mixture was flushed with argon and the mixture was run in the microwave oven for 45 minutes at 100° C. The solids were filtered off and washed with DCM. The solvent was evaporated and the crude product was partitioned between DCM and water. The organic phase was separated and dried with MgSO₄. The solvent was evaporated yielding the crude title compound which was used as such in the next step.

MS (ES−) m/z 526.6 [M−H]⁻.

### Example 55b

### (R)-tert-butyl 8-chloro-2-phenyl-2,3-dihydropyrido[3,2-f][1,4]oxazepine-4(5H)-carboxylate

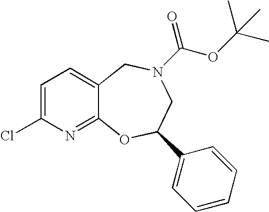

di-tert-butyl dicarbonate (615 mg, 2.82 mmol) in DCM was added to a solution of (R)-8-chloro-2-phenyl-2,3,4,5-tetrahydropyrido[3,2-f][1,4]oxazepine (Example 11b, 700 mg, 2.68 mmol) in DCM (5 mL) at 20° C. Evolution of gas started immediately and after 2 min stopped. The solvent was removed under reduced pressure yielding the title compound (1090 mg, 113%).

MS (ES−) m/z 359.1 [M−H]⁻.

### Example 56

### (R)—N-(6-Methoxy-5-(1-methyl-1H-pyrazol-4-yl)pyridin-2-yl)-2-phenyl-2,3,4,5-tetrahydropyrido[3,2-f][1,4]oxazepin-8-amine

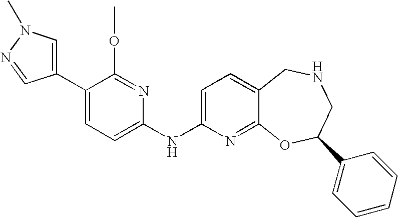

TFA (0.424 mL, 5.50 mmol) was added to a solution of (R)-tert-butyl 8-(6-methoxy-5-(1-methyl-1H-pyrazol-4-yl)pyridin-2-ylamino)-2-phenyl-2,3-dihydropyrido[3,2-f][1,4]oxazepine-4(5H)-carboxylate (375 mg, 0.71 mmol) in DCM (3 mL). The reaction mixture was heated to reflux and stirred over night. After cooling the reaction to room temperature the mixture was neutralised using triethylamine. The solvent was evaporated. The crude product was purified using flash chromatography, 0-10% MeOH in DCM yielding the title compound (101 mg, 33.2%).

¹H NMR (500 MHz, DMSO-d₆) δ ppm 9.87 (s, 1H) 8.03 (s, 1H) 7.88 (d, 1H) 7.84 (s, 1H) 7.77-7.82 (m, 2H) 7.53-7.57 (m, 2H) 7.47-7.52 (m, 2H) 7.43 (m, 1H) 6.99 (d, 1H) 5.37 (dd, 1H) 4.40 (m, 2H) 4.02 (s, 3H) 3.86 (s, 3H) 3.65-3.70 (m, 2H) MS (ES+) m/z 429.2 [M+H]⁺.

### Example 56a

### (R)-tert-butyl 8-(6-methoxy-5-(1-methyl-1H-pyrazol-4-yl)pyridin-2-ylamino)-2-phenyl-2,3-dihydropyrido[3,2-f][1,4]oxazepine-4(5H)-carboxylate

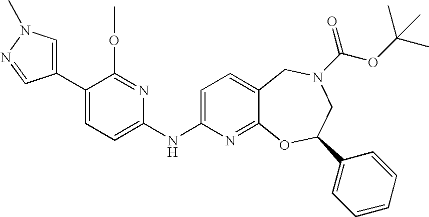

(R)-tert-butyl 8-chloro-2-phenyl-2,3-dihydropyrido[3,2-f][1,4]oxazepine-4(5H)-carboxylate (256 mg, 0.71 mmol), 6-methoxy-5-(1-methyl-1H-pyrazol-4-yl)pyridin-2-amine (145 mg, 0.71 mmol), cesium carbonate (416 mg, 1.28 mmol) and acetoxy(2′-(di-tert-butylphosphino)biphenyl-2-yl)palladium (16.43 mg, 0.04 mmol) were added to a microwave vial. 1,2-dimethoxyethane (2.5 mL) and EtOH (0.250 mL) were added. The reaction mixture was flushed with argon and the mixture was run in the microwave oven for 45 minutes at 100° C. The solids were filtered off and washed with DCM. The solvent was evaporated and the crude product was partitioned between DCM and water. The organic phase was separated and dried with MgSO₄. The solvent was evaporated yielding the crude title compound which was used as such in the next step.

MS (ES+) m/z 529.6 [M+H]⁺.

### Example 57

### (R)—N-(5-(1-Methyl-1H-pyrazol-4-yl)pyridin-2-yl)-2-phenyl-2,3,4,5-tetrahydropyrido[3,2-f][1,4]oxazepin-8-amine

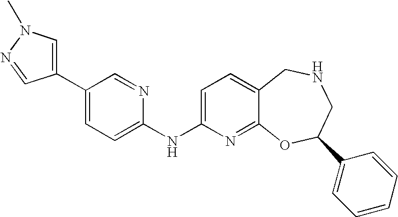

TFA (0.424 mL, 5.50 mmol) was added to a solution of (R)-tert-butyl 8-(5-(1-methyl-1H-pyrazol-4-yl)pyridin-2-ylamino)-2-phenyl-2,3-dihydropyrido[3,2-f][1,4]oxazepine-4(5H)-carboxylate (274 mg, 0.55 mmol) in DCM (3 mL). The reaction mixture was heated to reflux and stirred over night. After cooling the reaction to room temperature the mixture was neutralised using triethylamine. The solvent was evaporated. The crude product was purified using flash chromatography, 0-10% MeOH in DCM yielding the title compound (101 mg, 46.1%).

¹H NMR (500 MHz, DMSO-d₆) δ ppm 9.90 (s, 1H) 8.47 (s, 1H) 8.09 (s, 1H) 7.80-7.86 (m, 2H) 7.68-7.77 (m, 2H) 7.47 (m, 6H) 5.28 (dd, 1H) 4.30 (m, 2H) 3.86 (s, 3H) 3.55-3.64 (m, 2H). MS (ES+) m/z 399.2 [M+H]⁺.

### Example 57a

### (R)-tert-butyl 8-(5-(1-methyl-1H-pyrazol-4-yl)pyridin-2-ylamino)-2-phenyl-2,3-dihydropyrido[3,2-f][1,4]oxazepine-4(5H)-carboxylate

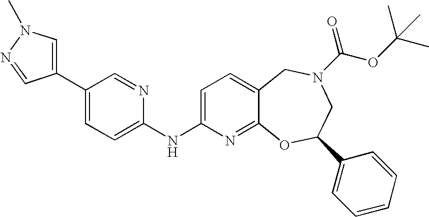

(R)-tert-butyl 8-chloro-2-phenyl-2,3-dihydropyrido[3,2-f][1,4]oxazepine-4(5H)-carboxylate (200 mg, 0.55 mmol), 5-(1-methyl-1H-pyrazol-4-yl)pyridin-2-amine (97 mg, 0.55 mmol), cesium carbonate (325 mg, 1.00 mmol) and acetoxy(2′-(di-tert-butylphosphino)biphenyl-2-yl)palladium (12.83 mg, 0.03 mmol) were added to a microwave vial. 1,2-dimethoxyethane (2.5 mL) and EtOH (0.250 mL) were added. The reaction mixture was flushed with argon and the mixture was run in the microwave oven for 45 minutes at 100° C. The solids were filtered off and washed with DCM. The solvent was evaporated and the crude product was partitioned between DCM and water. The organic phase was separated and dried with MgSO₄. The solvent was evaporated yielding the crude title compound which was used as such.

MS (ES+) m/z 499.6 [M+H]⁺.

### Example 57b

### 5-(1-Methyl-1H-pyrazol-4-yl)-pyridin-2-ylamine

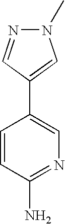

A mixture of 2-amino-5-bromopyridine (3.0 g, 17.3 mmol), 1-methyl-4-(4,4,5,5-tetramethyl-1,3,2-dioxaborolan-2-yl)-1H-pyrazole (5.4 g, 26.0 mmol) and potassium carbonate (3.6 g, 26.0 mmol) in dimethoxyethane (60 mL) and water (10 mL) was degassed for 15 minutes using nitrogen. [1,1′-Bis(di-tert-butylphosphino)-ferrocene]palladium (II) dichloride (562 mg, 0.87 mmol) was added and the reaction mixture was heated in a sealed tube at 90° C. overnight. The mixture was cooled to room temperature and partitioned between ethyl acetate and aqueous sodium bicarbonate solution. The organic phase was separated and the aqueous layer was re-extracted with ethyl acetate. The combined extracts were dried over magnesium sulfate, filtered and concentrated under reduced pressure. The residue was purified by flash column chromatography using a gradient of 2 to 5% methanol in dichloromethane. The desired fractions were collected and concentrated in vacuo, and the solid obtained was washed with diethyl ether to offord the title compound (2 g, 66%).

¹H NMR (400 MHz, CHLOROFORM-d) δ ppm 3.94 (s, 3H) 4.39 (br. s., 2H) 6.53 (d, J=8.21 Hz, 1H) 7.50 (m, 2H) 7.66 (s, 1H) 8.21 (d, J=2.34 Hz, 1H). ESMS m/z: [M+1]⁺175.1

### Example 58

### (R)-4-Methyl-N-(6-(1-methyl-1H-pyrazol-4-yl)pyridin-3-yl)-2-phenyl-2,3,4,5-tetrahydropyrido[3,2-f][1,4]oxazepin-8-amine

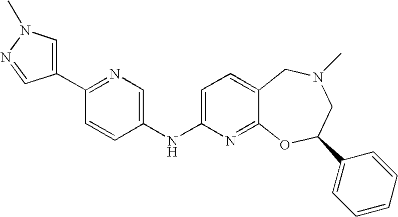

A reaction mixture of (R)-8-chloro-4-methyl-2-phenyl-2,3,4,5-tetrahydropyrido[3,2-f][1,4]oxazepine (0.200 g, 0.73 mmol), 6-(1-methyl-1H-pyrazol-4-yl)pyridin-3-amine (Example 54a, 0.127 g, 0.73 mmol), acetoxy(2′-(di-tert-butylphosphino)biphenyl-2-yl)palladium (0.017 g, 0.04 mmol) and cesium carbonate (0.356 g, 1.09 mmol) in DME (3 mL) was heated in the microwave oven at 100° C. for 60 minutes. The reaction mixture was filtrated through celite and rinsed with DCM. The solvent was evaporated and the crude product was purified by silica flash chromatography using a gradient of methanol (0 to 5%) in DCM affording the title compound (0.122 g, 40.6%).

¹H NMR (500 MHz, DMSO-d₆) δ ppm 9.20 (s, 1H), 8.69 (d, 1H), 8.10 (s, 1H), 8.01 (dd, 1H), 7.85 (s, 1H), 7.46-7.56 (m, 4H), 7.41 (t, 2H), 7.32-7.37 (m, 1H), 6.57 (d, 1H), 5.08 (t, 1H), 3.84 (s, 3H), 3.61-3.75 (m, 2H), 2.97 (d, 2H), 2.35 (s, 3H). MS (ES+) m/z 413 [M+H]⁺.

### Example 59

### (R)-4-Methyl-N-(4-(1-methyl-1H-pyrazol-4-yl)phenyl)-2-phenyl-2,3,4,5-tetrahydropyrido[3,2-f][1,4]oxazepin-8-amine

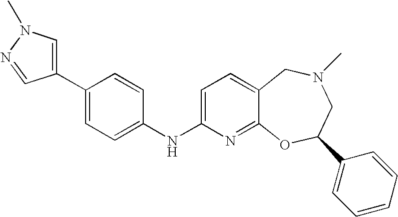

4-(1-Methyl-1H-pyrazol-4-yl)aniline (126 mg, 0.73 mmol), (R)-8-chloro-4-methyl-2-phenyl-2,3,4,5-tetrahydropyrido[3,2-f][1,4]oxazepine (200 mg, 0.73 mmol), Cesium carbonate (427 mg, 1.31 mmol) and acetoxy(2′-(di-tert-butylphosphino)biphenyl-2-yl)palladium (16.85 mg, 0.04 mmol) were added to a microwave vial. 1,2-dimethoxyethane (2 mL) and EtOH (0.222 mL) were added. The reaction mixture was flushed with argon and the mixture was run in the microwave oven for 30 minutes at 100° C. Additional acetoxy(2′-(di-tert-butylphosphino)biphenyl-2-yl)palladium (16.85 mg, 0.04 mmol) was added and the reaction was run for another 45 min in the microwave oven. The solids were filtered off and washed with DCM. The solvent was evaporated and the crude product was purified using straight phase chromatography, 0-10% MeOH in DCM, yielding the title compound (95 mg, 31.7%).

¹H NMR (500 MHz, DMSO-d₆) δ ppm 9.00 (s, 1H) 7.97 (s, 1H) 7.74 (s, 1H) 7.51-7.54 (m, 2H) 7.47-7.51 (m, 3H) 7.39-7.44 (m, 4H) 7.34 (s, 1H) 6.56 (d, 1H) 5.04-5.10 (m, 1H) 3.82 (s, 3H) 3.60-3.74 (m, 2H) 2.93-2.99 (m, 2H) 2.35 (s, 3H). MS (ES+) 412.2 [M+H]⁺.

### Example 59a

### 4-(1-methyl-1H-pyrazol-4-yl)-phenylamine

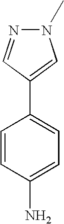

A mixture of 4-bromoaniline (3.0 g, 17.3 mmol), 1-methyl-4-(4,4,5,5-tetramethyl-1,3,2-dioxaborolan-2-yl)-1H-pyrazole (5.4 g, 26 mmol) and potassium carbonate (3.6 g, 26 mmol) in dimethoxyethane (60 mL) and water (10 mL) was degassed for 15 minutes using nitrogen. [1,1′-Bis(di-tert-butylphosphino)-ferrocene]palladium (II) dichloride (562 mg, 0.87 mmol) was added and the reaction mixture was heated in a sealed tube at 90° C. overnight. The mixture was cooled to room temperature and partitioned between ethyl acetate and aqueous sodium bicarbonate solution. The organic phase was separated and the aqueous layer was re-extracted with ethyl acetate. The combined extracts were dried over magnesium sulfate, filtered and concentrated under reduced pressure. The residue was triturated with diethyl ether and the separated red solid was filtered and washed with diethyl ether. The solid was further purified by flash column chromatography using a gradient of 50-60% ethyl acetate in hexane to yield the title compound (1.4 g, 47%).

¹H NMR (400 MHz, CHLOROFORM-d) δ ppm 3.66 (br. s., 2H) 3.91 (s, 3H) 6.70 (d, J=8.60 Hz, 2H) 7.27 (d, 2H) 7.49 (s, 1H) 7.66 (s, 1H). ESMS m/z: [M+1]⁺ 174.1.

### Example 60

### (R)-(1-(4-(4-Methyl-2-phenyl-2,3,4,5-tetrahydropyrido[3,2-f][1,4]oxazepin-8-ylamino)phenyl)-1H-imidazol-2-yl)methanol

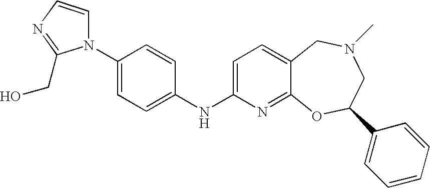

(1-(4-Aminophenyl)-1H-imidazol-2-yl)methanol (138 mg, 0.73 mmol), (R)-8-chloro-4-methyl-2-phenyl-2,3,4,5-tetrahydropyrido[3,2-f][1,4]oxazepine (200 mg, 0.73 mmol), Cesium carbonate (427 mg, 1.31 mmol) and acetoxy(2′-(di-tert-butylphosphino)biphenyl-2-yl)palladium (16.85 mg, 0.04 mmol) were added to a microwave vial. 1,2-dimethoxyethane (2 mL) and EtOH (0.222 mL) were added. The reaction mixture was flushed with argon and the mixture was run in the microwave oven for 30 minutes at 100° C. Additional acetoxy(2′-(di-tert-butylphosphino)biphenyl-2-yl)palladium (16.85 mg, 0.04 mmol) was added and the reaction was again heated to 100° C. for 45 min. The solids were filtered off and washed with DCM. The solvent was evaporated and the crude product was purified twice using straight phase chromatography, 0-10% MeOH in DCM, yielding the title compound (63.0 mg, 20.24%).

¹H NMR (500 MHz, DMSO-d₆) δ ppm 9.25 (s, 1H) 7.65-7.70 (m, 2H) 7.54 (d, 1H) 7.46-7.50 (m, 2H) 7.31-7.44 (m, 5H) 7.29 (d, 1H) 6.94 (d, 1H) 6.61 (d, 1H) 5.29 (t, 1H) 5.08 (s, 1H) 4.34 (dd, 2H) 3.68 (d, 2H) 2.97 (d, 2H) 2.35 (s, 3H). MS (ES+) m/z 428.2 [M+H]⁺.

### Example 60a

### [1-(4-Amino-phenyl)-1H-imidazol-2-yl]methanol

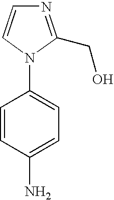

A suspension of [1-(4-nitrophenyl)-1H-imidazol-2-yl]-methanol (2.77 g, 12.66 mmol) and Pd/C (10%, 280 mg) in a mixture of EtOH (50 mL) and THF (5 mL) was shaken under a hydrogen atmosphere (35 psi) for 3 days at room temperature. The reaction mixture was filtered through a pad of Celite and concentrated under reduced pressure to afford the title compound (2.35 g) that required no further purification.

¹H NMR (400 MHz, DMSO-d₆): 6 ppm 4.28 (d, 1H) 5.24-5.18 (m, 1H) 5.31 (s, 2H) 6.63-6.58 (m, 2H) 6.90 (d, 1H) 7.13-7.08 (m, 2H) 7.16 (d, 1H). ESMS m/z: 190.2 [M+1]⁺.

### Example 60b

### [1-(4-Nitrophenyl)-1H-imidazol-2-yl]methanol

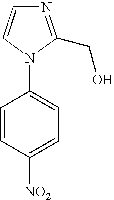

NaBH₄ (1.22 g, 32.1 mmol) was added in small portions over 15 minutes to a vigorously stirred suspension of 1-(4-nitrophenyl)-1H-imidazole-2-carbaldehyde 3 (12.81 g, 59.0 mmol) in anhydrous MeOH (350 mL) at 0° C. The reaction mixture was stirred for 30 minutes (TLC using 5% MeOH in CH₂Cl₂ indicated complete conversion) and H₂O (100 mL) was added. The mixture was concentrated to approximately 20% of the original volume and additional H₂O (400 mL) was added. The precipitated solid was filtered, washed with water and dried in vacuo to afford the title compound (11.85 g) which was used in the next step without further purification.

¹H NMR (400 MHz, CDCl₃): δ ppm 4.65 (s, 2H) 5.15-4.90 (br.s., 1H) 7.17 (s, 1H) 7.19 (s, 1H) 7.81-7.76 (m, 2H) 8.43-8.38 (m, 2H)

### Example 60c

### 1-(4-Nitrophenyl)-1H-imidazole-2-carbaldehyde

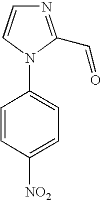

K₂CO₃ (14.35 g, 104.0 mmol) was added to a mixture of 1-fluoro-4-nitrobenzene 1 (8.48 mL, 80.0 mmol) and 1H-imidazole-2-carbaldehyde (8.45 g, 88.0 mmol) in dry DMF (75 mL) at room temperature under a nitrogen atmosphere. The reaction mixture was heated at 80° C. for 1.5 hours. The reaction mixture was allowed to cool to room temperature and H₂O (400 mL) was added with vigorous stirring. The resultant precipitate was filtered, washed with H₂O (5×100 mL) and dried in vacuo to afford the tile compound 3 (12.81 g, 74% yield) which was used in the next step without further purification.

¹H NMR (400 MHz, CDCl₃): δ ppm 7.32 (s, 1H) 7.49 (s, 1H) 7.58-7.53 (m, 2H) 8.40-8.36 (m, 2H) 9.86 (s, 1H)

### Assays

The level of activity of the compounds on Aβ formation was tested using the following method:

Compounds were diluted in 100% DMSO and stored at 20° C. prior to use. Human Embryonic Kidney (HEK) cell line stably expressing APP with the Swedish mutation (APPswe) were cultured using Dulbecco's Modified Eagles medium (DMEM) supplied with 4500 g/l glucose, Na-pyruvate and GlutaMAX with 10% FBS, 100 U/ml penicillin-streptomycin (PEST) respectively, 1×non-essential amino acids (NEAA), 10 μM Hepes, 100 μg/ml Zeocine. Cells at about 80% confluence were washed with PBS, detached from culture flasks using 1×Trypsin/EDTA diluted in PBS, re-suspended in cell media and plated in 384-well poly-d-lysine coated cell culture plates at about 10000-15000 cells/well, in 25 μL cell media. Optionally, cryo-preserved cells (frozen and stored at −140° C. in 90% cell media and 10% DMSO) were thawed, washed and plated as above. Next the cells were incubated for 15-24 h at 37° C. and 5% CO₂, after which cell medium was changed. Fresh medium containing test compound diluted ×200 from prepared compound plate was added to the cells before further incubation for 4-6 hours at 37° C. and 5% CO₂. After incubation with test compound the amount of Aβ peptides, including Aβ42, Aβ40, Aβ39, Aβ38 and Aβ37, secreted to cell medium was analyzed using the electrochemiluminescence assay technology from Meso Scale Discovery Technology, in combination with specific antibodies raised against the different Aβ peptides. Potential cytotoxic effects of the compounds were usually assayed by measuring the ATP content (ViaLight) from cell lysate.

### RESULTS

Typical IC₅₀ values of Aβ42 release for the compounds of the present invention are in the range of about 1 to about 16000 nM. Biological data on final compounds are given below in Table 1.

TABLE 1
pIC₅₀ values of Aβ42 release for
the compounds of the present invention

| Example | pIC50 |
|---|---|
| 1 | 5.8 |
| 2 | 5.9 |
| 3 | 6.4 |
| 4 | 6.7 |
| 5 | 6.0 |
| 6 | 5.2 |
| 7 | 7.4 |
| 8 | 6.8 |
| 9 | 8.1 |
| 10 | 7.9 |
| 11 | 7.8 |
| 12 | 7.8 |
| 13 | 7.7 |
| 14 | 7.7 |
| 15 | 7.5 |
| 16 | 7.1 |
| 17 | 7.1 |
| 18 | 7.0 |
| 19 | 6.8 |
| 20 | 6.7 |
| 21 | 6.7 |
| 22 | 6.3 |
| 23 | 6.3 |
| 24 | 6.1 |
| 25 | 5.3 |
| 26 | 4.7 |
| 27 | 5.1 |
| 28 | 5.2 |
| 29 | 5.9 |
| 30 | 6.2 |
| 31 | 6.2 |
| 32 | 6.3 |
| 33 | 6.5 |
| 34 | 6.6 |
| 35 | 6.7 |
| 36 | 6.8 |
| 37 | 6.9 |
| 38 | 7.0 |
| 39 | 7.1 |
| 40 | 7.3 |
| 41 | 7.4 |
| 42 | 7.5 |
| 43 | 7.5 |
| 44 | 7.5 |
| 45 | 7.6 |
| 46 | 7.6 |
| 47 | 7.8 |
| 48 | 7.0 |
| 49 | 7.0 |
| 50 | 8.1 |
| 51 | 6.3 |
| 52 | 6.4 |
| 53 | 7.3 |
| 54 | <5.5 |
| 55 | 7.5 |
| 56 | 8.0 |
| 57 | 6.9 |
| 58 | 6.5 |
| 59 | 7.1 |
| 60 | 7.5 |

1-28. (canceled)

29. A compound of the following formula 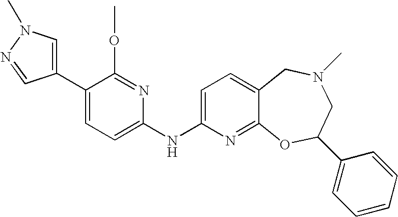
or a pharmaceutically acceptable salt thereof.

30. A compound of claim 1 of the following formula 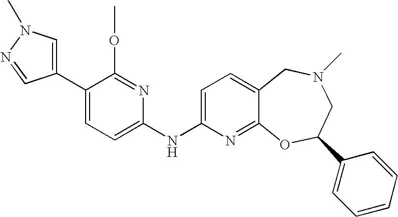
or a pharmaceutically acceptable salt thereof.

31. A pharmaceutical composition comprising an effective amount of a compound, or a pharmaceutically acceptable salt thereof, according to claim 1 or 2, in association with a pharmaceutically acceptable excipient, carrier or diluents.
